(19) United States
(12) Patent Application Publication
(10) Pub. No.: US 2025/0283138 A1
(43) Pub. Date: Sep. 11, 2025

(54) BIOREACTIVE COMPOUNDS AND METHODS OF USE THEREOF

(71) Applicant: The Regents of the University of California, Oakland, CA (US)

(72) Inventors: Lei Wang, San Francisco, CA (US); Jun Liu, San Francisco, CA (US)

(21) Appl. No.: 18/279,463

(22) PCT Filed: Mar. 1, 2022
(86) PCT No.: PCT/US2022/018381

§ 371 Date: Aug. 30, 2023

Related U.S. Application Data
(60) Provisional application No. 63/155,222, filed on Mar. 1, 2021.
(60) Provisional application No. 63/214,432, filed on Jun. 24, 2021.

(51) Int. Cl. C12P 21/02; C07C 309/77; C07C 309/89; C07C 311/47; C07D 233/64; C12N 9/00

(52) U.S. Cl. CPC C12P 21/02; C07C 309/77; C07C 309/89; C07C 311/47; C07D 233/64; C12N 9/93; C12Y 601/01026

(57) ABSTRACT

Provided herein are, inter alia, bioreactive unnatural amino acids, compounds containing the unnatural amino acids, and methods of using same.

### RELATED APPLICATIONS

This application claims priority to U.S. Application No. 63/214,432 filed Jun. 24, 2021, and U.S. Application No. 63/155,222 filed Mar. 1, 2021, the disclosures of which are incorporated by reference herein in their entirety.

### STATEMENT AS TO RIGHTS TO INVENTIONS MADE UNDER FEDERALLY SPONSORED RESEARCH AND DEVELOPMENT

This invention was made with government support under grant no. R01 GM118384 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-709001WO_SL_ST25.txt, created on Feb. 28, 2022, 55,165 bytes, machine format IBM-PC, MS Windows operating system, is hereby incorporated by reference.

### BACKGROUND

Introducing new chemical bonds into proteins provides innovative avenues for manipulating protein structure and function. Unnatural amino acids (Uaas) containing diverse latent bioreactive functional groups have recently been introduced into proteins via genetic code expansion. This offers an exquisite tool not only to study cellular protein interactions but also create novel protein-based therapeutics. SuFEx click chemistry via the latent aryl fluorosulfate group has demonstrated value in aiding modular organic synthesis, chemical biology, and drug development. As set forth in US Publication No. 2021/0002325, the inventors incorporated fluorosulfate-L-tyrosine (FSY) into proteins for protein crosslinking and generating covalent protein drugs.

There is a need in the art, inter alia, for new and other unnatural amino acids that can be used for protein identification, drug target discovery, or biotherapeutics. Provided herein are, inter alia, solutions to these and other needs in the art.

### BRIEF SUMMARY

Provided herein is fluorosulfonyloxybenzoyl-L-lysine (FSK) having the structure of Formula (A):

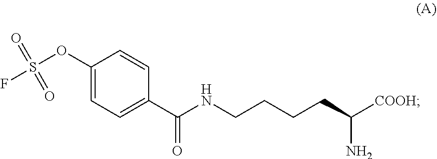 [SMILES: N[C@@H](CCCCNC(=O)c1ccc(OS(=O)(=O)F)cc1)C(=O)O]

Provided herein are biomolecules having the structure of Formula (B):

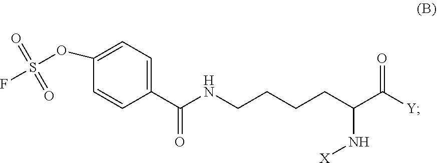

wherein: (i) X comprises at least one amino acid, and Y is OH; (ii) Y comprises at least one amino acid and X is H; or (iii) X and Y each comprise at least one amino acid.

Provided herein are biomolecules having the structure of Formula (C):

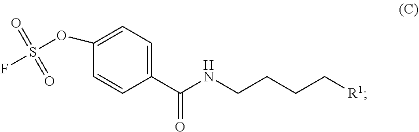

wherein R¹ is a biomolecule. In aspects, R¹ is a peptidyl moiety, a nucleic acid moiety, a carbohydrate moiety, or a small molecule. In aspects, R¹ is a peptidyl moiety.

Provided herein are biomolecule conjugates comprising a first biomolecule moiety conjugated to a second biomolecule moiety through a bioconjugate linker, wherein the bioconjugate linker has the structure of Formula (D):

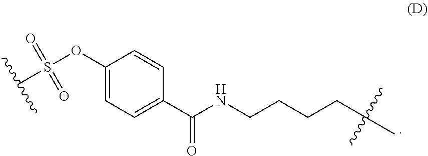 [SMILES: *CCCCNC(=O)c1ccc(OS(*)(=O)=O)cc1]

Provided herein are biomolecule conjugates having the structure of Formula (E):

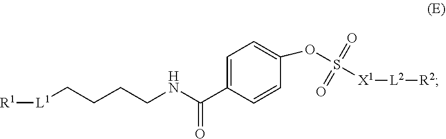

wherein R¹ is a first biomolecule moiety; R² is a second biomolecule moiety; and L¹, L², and X¹ are as defined herein.

Provided herein are biomolecules comprising FSK, wherein FSK has a side chain having the structure of Formula (F):

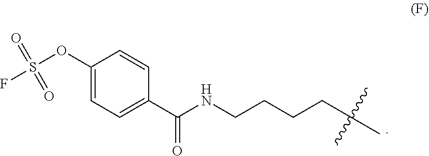 [SMILES: *CCCCNC(=O)c1ccc(OS(=O)(=O)F)cc1]

In aspects, the biomolecule is a protein. In aspects, the protein is an antibody, an antibody variant (e.g., an antigen-binding fragment, a single-domain antibody, a single-chain variable fragment, an affibody), or a membrane receptor.

These and other embodiments and aspects of the disclosure are described in more detail herein.

### BRIEF DESCRIPTION OF THE DRAWINGS

FIGS. 1A-1C show site-specific incorporation of FSK into proteins. FIG. 1A: Schematic illustration of FSK incorporation into proteins via genetic code expansion. FIG. 1B: SDS-PAGE of purified ubiquitin (6FSK). FIG. 1C: Mass spectrum of the intact ubiquitin (6FSK). Theoretical molecular weight: 9589.9 Da; observed: 9590.1 Da.

FIGS. 2A-2F show genetically encoded FSK enables protein crosslinking at long distance. FIG. 2A: Chemical structures of FSY and FSK, and schematic illustration of aryl fluorosulfate reacting with nucleophilic residues (e.g., Lys, Tyr, His) in proximity via SuFEx chemistry. FIG. 2B: Reaction distances of FSY and FSK measured from the Cα to the F atom at their energy minimized states: 9.0 angstroms (FSY) and 13.8 angstroms (FSK). FIG. 2C: Crystal structure of ecGST (PDB code: 1A0F) showing the distances between Glu65 and the adjacent nucleophilic residues in yellow dotted lines. FIG. 2D: Western blot analysis of ecGST dimeric crosslinking induced by FSK or FSY incorporated at site 65 of ecGST. FIG. 2E: Crystal structure of sjGST (PDB code: 1Y6E) showing the distance between the Cαs of Lys 44 and Ala97). FIG. 2F: Western blot analysis of sjGST dimeric crosslinking induced by FSK or FSY. * indicates other proteins interacting with sjGST in E. coli.

FIGS. 3A-3B show FSK mediated intramolecular covalent crosslinking in ubiquitin. FIG. 3A: Structure of ubiquitin (PDB code: 1AAR) showing Glu18 for FSK incorporation to target Lys29. FIG. 3B: ESI-MS of Ub(18FSK). The peak of 9587.9 Da corresponds to the intact Ub(18FSK) (calculated MW: 9587.9 Da). The peak of 9568.6 Da corresponds to the intramolecularly cross-linked Ub via FSK18 reacting with Lys29 and losing HF (calculated MW: 9567.9). The peak 9506.8 Da corresponds to Ub(18FSK) losing SO₂F (calculated MW: 9506.9), which could be due to the impurity of FSK. The tandem mass spectrum (not shown) of the cross-linked peptide identified from the trypsin-digested Ub(18FSK) showed that FSK reacted with Lys29 as designed.

FIGS. 4A-4F show FSK enabled 7D12 nanobody to covalently target the EGFR receptor. FIG. 4A: Structure of nanobody 7D12 in complex with EGFR (PDB code: 4KRL), showing Arg30 and Ser31 of 7D12 for FSK incorporation to target His359 of EGFR. FIG. 4B: ESI-MS analysis of 7D12(31FSK). Calculated MW: 14673.1 Da (forming 1 pair of disulfide bond); measured MW: 14673.2 Da. FIG. 4C: SDS-PAGE analysis of covalent crosslinking of nanobody 7D12 with EGFR in vitro. FIG. 4D: Western blot analysis of covalent crosslinking of nanobody 7D12 with EGFR in vitro. Only 7D12(31FSK) cross-linked EGFR covalently. FIG. 4E: Western blot analysis of nanobody 7D12 crosslinking with native EGFR expressed on A431 cell surface. 7D12 and 7D12(31FSK) were incubated with A431 cells at indicated time interval, and cell lysates were immunoblotted with anti-His antibody to detect the nanobody 7D12. FIG. 4F: schematic of the distance between 7D12 (Tyr109) and EGFR (Lys443), where the Lys 443 was shown as a state after site mutagenesis in PDB structure 4KRL.

FIGS. 5A-5C show genetically incorporation of FSK into proteins for protein crosslinking in mammalian cells. FIG. 5A: Fluorescence microscopic images of HeLa-EGFP(182TAG) reporter cells under different conditions. Cells were transfected with or without pNEU-FSKRS, and grew with or without 1 mM FSK. Top: bright field; bottom: GFP fluorescence channel. FIG. 5B: Western blot analysis of FSK incorporation into EGFP in HeLa cells. Samples from (a) were lysed and detected using an anti-GFP antibody. GAPDH expression level was used as reference. FIG. 5C: Western blot analysis of FSK-mediated ecGST crosslinking in mammalian cells. pNEU-FSKRS was co-transfected with pCDNA3.1-ecGST(WT), ecGST(86TAG), or ecGST(86TAG/92A) into HEK293T cells. The dimeric crosslinking of ecGST was detected using an anti-His antibody. GAPDH was used as a reference.

FIGS. 6A-6B show primers used for cloning as described in the Examples.

FIG. 7 compares the FSK incorporation efficiency of the FSKRS' mutants inducing at 18° C. for 24 hr.

FIG. 8 compares the FSK incorporation efficiency of the FSKRS' mutants inducing at 30° C. for 6 hr.

FIG. 9 shows incorporation of FSK into EGFP (182TAG) detected by Western blot. FSKRS was co-transformed into pBAD-EGFP(182TAG), protein expression was induced with or without 1 mM FSK. The successful incorporation of FSK into EGFP was detected by running Western blot using anti-his antibody.

FIG. 10 shows incorporation of FSK into sfGFP (2TAG) and sfGFP(151TAG). pEVOL-FSKRS was co-transformed with pBAD-sfGFP(2TAG) and pBAD-sfGFP(151TAG) into DH10b cells respectively. Protein expression was induced with or without 1 mM FSK. The successful incorporation of FSK into EGFP was detected by a plate reader (485 nm excitation wavelength, 528 nm emission wavelength). The plot represented the value after normalization to bacterial growth at optical density 600 nm.

FIG. 11 compares the FSY and FSK mediated GST crosslinking in short distance proximity. The pEVOL-FSYRS and pEVOL-FSKRS was co-expressed with ecGST 103TAG/107Ala, GST 103TAG/107His, GST 103TAG/107Lys, GST 103TAG/107Tyr respectively and induced in the presence of 1 mM FSK or FSY at 37° C. for 6 hr. The WT GST was used as a negative control. The GST dimer crosslinking was detected by Western blot by using anti-His antibody.

FIGS. 12A-12B compare the FSY and FSK mediated E. coli GST crosslinking at the 86th position. FIG. 12A is a schematic of the FSY/FSK crosslinking at Va186. FIG. 12B show the reulst of the pEVOL-FSYRS and pEVOL-FSKRS co-expressed with ecGST WT or pBAD-GST (86TAG) in the presence of 1 mM FSK or FSY at 37° C. for 6 hr. The WT GST was used as a negative control. The GST dimer crosslinking was detected by Western blot by using anti-His antibody.

FIG. 13 compares the crosslinking efficiency of FSK and FSY in mediating Trx and CysH crosslinking.

FIG. 14 shows purification of 7D12 (30FSK) and 7D12 (31FSK) in the presence and absence of 1 mM FSK during expression.

FIG. 15 shows utilization of 7D12 (30FSK), 7D12 (30FSY), 7D12 (109FSK) and 7D12 (109FSY) for crosslinking with EGFR in vitro.

FIG. 16 shows the utilization of Trx (59FSK), Trx (62FSK), Trx (59FSY), Trx (62FSY), for crosslinking with unknown substrates in vivo.

FIG. 17 is a schematic illustration of using FSK or FSY to identify Trx substrate proteins through genetically encoded chemical crosslinking in live cells.

FIG. 18 shows the scheme for the synthesis of fluorosulfonyloxybenzoyl-L-lysine (FSK).

FIG. 19 shows the incorporation of FSK into sfGFP(151TAG) using different FSKRS in the absence of FSK in the media or in the presence of 1 mM FSK in the media (where+indicates the presence of 1 mM FSK in the media). Cells were grown at 37° C. and induced for 5.5 h. sfGFP fluorescence intensity was measured and normalized to cell optical density. NThis means Hisx6 was appended at the N-terminus; CThis means Hisx6 was appended at the C-terminus.

FIG. 20 shows the incorporation of FSK into sfGFP(151TAG) using different FSKRS in the absence and presence of 1 mM FSK in the media. Cells were grown at 18° C. and induced for 24 h, followed with fluorescence intensity measurement and OD normalization.

FIGS. 21A-21B show a Western Blot analysis of covalent crosslinking of nanobody 7D12 with EGFR in vitro, wherein nanobody 7D12 contained FSY at position 109, 113, and 116 (FIG. 21A) or FSY at position 1, 109, or 113 (FIG. 21B). Nanobody 7D12 was incubated with 500 nM EGFR in 15 ul PBS at 37° C. for 20 hours. Nanobody 7D12 is set forth as SEQ ID NO:88.

FIGS. 22A-22B show a SDS Page analysis (FIG. 22A) and a Western Blot analysis (FIG. 22B) of covalent crosslinking of nanobody 7D12 with EGFR in vitro, wherein nanobody 7D12 contained FSY at position 109. 2 μM of purified nanobody 7D12 was incubated with 500 nM EGFR in 15 ul PBS at 37° C. for 20 hours.

FIG. 23 is a Western Blot analysis of covalent crosslinking of nanobody 7D12 WT or nanobody 7D12 containing FSY at position 109 with the A431 cell line.

FIGS. 24A-24B show a SDS Page analysis (FIG. 24A) and a Western Blot analysis (FIG. 24B) of covalent crosslinking of nanobody 7D12 with EGFR in vitro, wherein nanobody 7D12 contained FSK at position 30 or position 31, or wherein nanobody 7D12 contained FSY at position 109. 2 μM of purified nanobody 7D12 was incubated with 500 nM EGFR in 15 ul PBS at 37° C. for 20 hours.

FIGS. 25A-25B show a SDS Page analysis (FIG. 25A) and a Western Blot analysis (FIG. 25B) of covalent crosslinking of nanobody 7D12 with EGFR in vitro, wherein nanobody 7D12 contained FSK at position 31, or wherein nanobody 7D12 contained FSY at position 109. 2 μM of purified nanobody 7D12 was incubated with 500 nM EGFR in 15 ul PBS at 37° C. for 20 hours.

### DETAILED DESCRIPTION

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) may be placed at any interior position of the heteroalkyl group or at the position at which the alkyl group is attached to the remainder of the molecule. Heteroalkyl is an uncyclized chain. Examples include, but are not limited to —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom. A heteroalkyl moiety may include two optionally different heteroatoms. A heteroalkyl moiety may include three optionally different heteroatoms. A heteroalkyl moiety may include four optionally different heteroatoms. A heteroalkyl moiety may include five optionally different heteroatoms. A heteroalkyl moiety may include up to 8 optionally different heteroatoms. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In aspects, monocyclic ring systems are cyclic hydrocarbon groups containing from 3 to 8 carbon atoms, where such groups can be saturated or unsaturated, but not aromatic. In aspects, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In aspects, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In aspects, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In aspects, the bridged or fused bicyclic cycloalkyl is attached to the parent molecular moiety through any carbon atom contained within the monocyclic cycloalkyl ring. In aspects, cycloalkyl groups are optionally substituted with one or two groups which are independently oxo or thia. In aspects, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In aspects, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In aspects, the multicyclic cycloalkyl is attached to the parent molecular moiety through any carbon atom contained within the base ring. In aspects, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In aspects, a cycloalkenyl is a monocyclic, bicyclic, or a multicyclic cycloalkenyl ring system. In aspects, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In aspects, bicyclic cycloalkenyl rings are bridged monocyclic rings or a fused bicyclic rings. In aspects, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In aspects, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In aspects, the bridged or fused bicyclic cycloalkenyl is attached to the parent molecular moiety through any carbon atom contained within the monocyclic cycloalkenyl ring. In aspects, cycloalkenyl groups are optionally substituted with one or two groups which are independently oxo or thia. In aspects, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In aspects, the multicyclic cycloalkenyl is attached to the parent molecular moiety through any carbon atom contained within the base ring. In aspects, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In aspects, heterocyclyl groups are optionally substituted with one or two groups which are independently oxo or thia. In certain aspects, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In aspects, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ,” or “-” denotes the point of attachment of a chemical moiety to the remainder of a molecule or chemical formula.

The term “oxo” means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In aspects, the alkylarylene group has the formula:

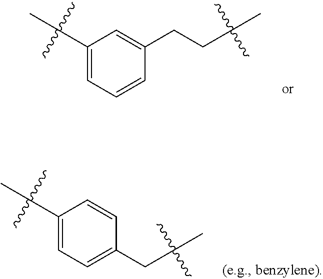 [SMILES: *CCc1cccc(*)c1 ; *Cc1ccc(*)cc1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In aspects, the alkylarylene moiety is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R′″, —CN, —NO₂, —NR′SO₂R″, —NR′C(O)R″, —NR′C(O)—OR″, —NR′OR″, in a number ranging from zero to (2 m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties: (A) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (B) alkyl, heteroalkyl, cycloalkyl, heterocycloalkyl, aryl, heteroaryl, substituted with at least one substituent selected from: (i) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (ii) alkyl, heteroalkyl, cycloalkyl, heterocycloalkyl, aryl, heteroaryl, substituted with at least one substituent selected from: (a) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, in aspects, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In aspects, at least one or all of these groups are substituted with at least one size-limited substituent group. In aspects, at least one or all of these groups are substituted with at least one lower substituent group.

In embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In aspects of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In aspects, each substituted or unsubstituted alkylene is a substituted or unsubstituted C₁-C₈alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In aspects, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In aspects, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In aspects, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In aspects, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In aspects, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²p, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²p, ³³p, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71).

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. y way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides may be referred to by their commonly accepted single-letter codes.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In aspects, the amino acid side chain may be a non-natural amino acid side chain. In aspects, the amino acid side chain is H,

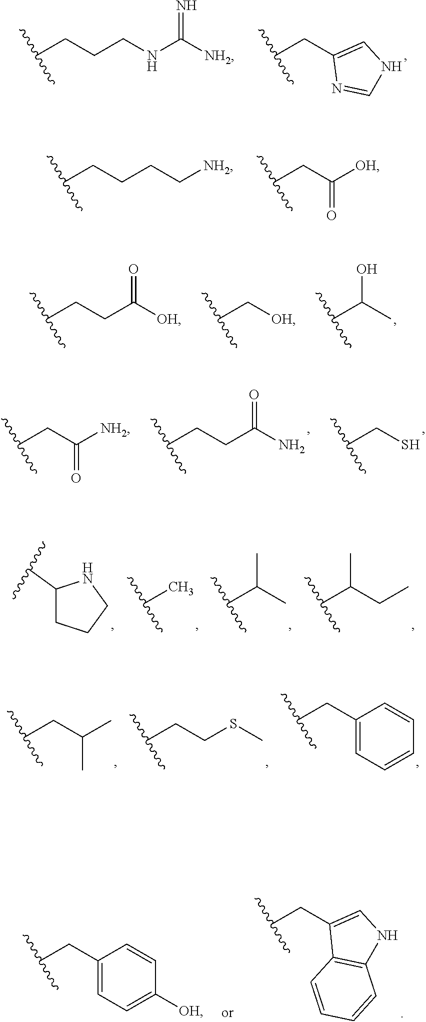

In embodiments, the unnatural amino acid side chain is

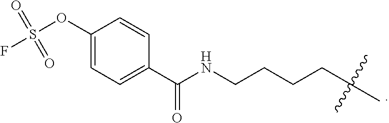 [SMILES: *CCCCNC(=O)c1ccc(OS(=O)(=O)F)cc1]

The term “non-natural amino acid side chain” or “unnatural amino acid side chain” or “Uaa” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]hept-5-ene-2-carboxylic acid hydrochloride, cis-2-aminocycloheptane-carboxylic acid hydrochloride, cis-6-Amino-3-cyclohexene-1-carboxylic acid hydrochloride, cis-2-amino-2-methylcyclohexanecarboxylic acid hydrochloride, cis-2-amino-2-methylcyclopentane-carboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-3-(2-quinolyl)-Ala-OH, N-Boc-1,2,3,6-tetrahydro-2-pyridinecarboxylic acid, Boc-R-(4-thiazolyl)-Ala-OH, Boc-3-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine. In embodiments, the unnatural amino acid is fluorosulfonyloxybenzoyl-L-lysine (FSK) having the following formula:

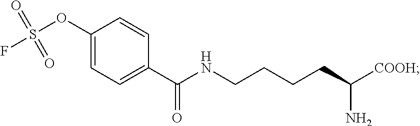 [SMILES: N[C@@H](CCCCNC(=O)c1ccc(OS(=O)(=O)F)cc1)C(=O)O]

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M). (see, e.g., Creighton, Proteins (1984)).

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to Tyr126 of the PylRS protein of SEQ ID NO:1 when the selected residue occupies the same essential spatial or other structural relationship as Tyr126 in the PylRS protein of SEQ ID NO:1. In embodiments, where a selected protein is aligned for maximum homology with the PylRS protein, the position in the aligned selected protein aligning with Tyr126 is said to correspond to Tyr126. Instead of a primary sequence alignment, a three dimensional structural alignment can also be used, e.g., where the structure of the selected protein is aligned for maximum correspondence with the PylRS protein and the overall structures compared. In this case, an amino acid that occupies the same essential position as Tyr126 in the structural model is said to correspond to the Tyr126 residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., ncbi.nlm.nih.gov/BLAST/or the like). Such sequences are then the to be “substantially identical.” This definition also refers to, or may be applied to, the compliment of a test sequence. The definition also includes sequences that have deletions and/or additions, as well as those that have substitutions. As described below, the preferred algorithms can account for gaps and the like. In embodiments, identity exists over a region that is at least about 25 amino acids or nucleotides in length, or more preferably over a region that is 50-100 amino acids or nucleotides in length.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}—C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (e.g., McCafferty et al., Nature 348:552-554 (1990)).

Antibodies are large, complex proteins with an intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat et al, Sequences of Proteins of Immunological Interest, U.S. Department of Health and Human Services, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

An “antibody variant” as provided herein refers to a polypeptide capable of binding to a receptor protein or an antigen and including one or more structural domains of an antibody or fragment thereof. Non-limiting examples of antibody variants include single-domain antibodies (nanobodies), affibodies (polypeptides smaller than monoclonal antibodies (e.g., about 6kDA) and capable of binding receptor proteins or antigens with high affinity and imitating monoclonal antibodies), an antigen-binding fragment (Fab), Fab dimer (monospecific Fab₂, bispecific Fab₂), trispecific Fab₃, monovalent IgGs, single-chain variable fragments (scFv), bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO 97/49805 and WO 97/49805, which are incorporated, by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

A “single-domain antibody” or “nanobody” refers to an antibody fragment having a single monomeric variable antibody domain. Like a whole antibody, it is able to bind selectively to a specific antigen. In embodiments, the single domain antibody is a human or humanized single-domain antibody.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The term “antigen” as provided herein refers to molecules capable of binding to the antibody binding domain provided herein. An “antigen binding domain” as provided herein is a region of an antibody that binds to an antigen (epitope). As described above, the antigen binding domain may include one constant and one variable domain of each of the heavy and the light chain (VL, VH, CL and CH1, respectively). In embodiments, the antigen binding domain includes a light chain variable domain and a heavy chain variable domain. In embodiments, the antigen binding domain includes light chain variable domain and does not include a heavy chain variable domain and/or a heavy chain constant domain. The paratope or antigen-binding site is formed on the N-terminus of the antigen binding domain. The two variable domains of an antigen binding domain may bind the epitope of an antigen.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Antibodies, e.g., recombinant, monoclonal, or polyclonal antibodies, can be prepared by many techniques known in the art (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. In embodiments, the antibodies described herein include humanized and/or chimeric monoclonal antibodies.

The phrase “specifically (or selectively) binds” to an antibody or an antigen or “specifically (or selectively) immunoreactive with” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

“Receptor protein” or “membrane receptor” refers to a receptor (protein) that is embedded in the plasma membrane of a cell. In embodiments, the receptor protein is located in the extracellular domain of a cell, the transmembrane domain of a cell, or the intracellular domain of a cell. In embodiments, the receptor protein is a cell-surface receptor. In embodiments, the receptor protein is in the extracellular domain. In embodiments, the receptor protein is in the transmembrane domain. In embodiments, the receptor protein is an ion channel-linked receptor, an enzyme-linked receptor, or a G protein-coupled receptor. In embodiments, the receptor protein is a hormone receptor.

The term “biomolecule” as used herein refers to large macromolecules such as, for example, proteins, carbohydrates, lipids, and nucleic acids, as well as small molecules such as, for example, primary and secondary metabolites. In aspects, the “biomolecule” refers to a protein. In aspects, “biomolecule” refers to a nucleic acid. In aspects, the “biomolecule” refers to a carbohydrate. In embodiments, the protein is a single-domain antibody. In embodiments, the protein is a membrane receptor.

The term “biomolecule moiety” refers to a peptidyl moiety, a carbohydrate moiety, a lipid moiety, or a nucleic acid moiety that forms a biomolecule.

The term “peptidyl moiety” as used herein refers to a protein, protein fragment, or peptide that may form part of a biomolecule or a biomolecule conjugate. In aspects, the peptidyl moiety forms part of a biomolecule (e.g., protein). In aspects, the peptidyl moiety forms part of a biomolecule (e.g., protein) conjugate. The peptidyl moiety may also be substituted with additional chemical moieties (e.g., additional R substituents). In aspects, the peptidyl moiety forms part of a single-domain antibody. In aspects, the peptidyl moiety forms part of a membrane receptor.

The term “amino acid moiety” as used herein refers to a monovalent amino acid, such that the amino acid can be linked to another compound or moiety, such as the compound of Formula (B) described herein.

The term “carbohydrate moiety” as used herein refers to carbohydrates, for example, polyhydroxy aldehydes, ketones, alcohols, acids, their simple derivatives and their polymers having linkages of the acetal type, that may form part of a biomolecule or a biomolecule conjugate. In aspects, the carbohydrate moiety forms part of a biomolecule. In aspects, the carbohydrate moiety forms part of a biomolecule conjugate. The carbohydrate moiety may also be substituted with additional chemical moieties (e.g., additional R substituents).

The term “nucleic acid moiety” as used herein refers to nucleic acids, for example, DNA, and RNA, that may form part of a biomolecule or biomolecule conjugate. In aspects, the nucleic acid moiety forms part of a biomolecule. In aspects, the nucleic acid moiety forms part of a biomolecule conjugate. The nucleic acid moiety may also be substituted with additional chemical moieties (e.g., additional R substituents).

A “small molecule” is a low molecular weight organic compounds, having a molecular weight of 10,000 Daltons or less, of natural or synthetic nature. Attachments to small molecules could occur through any covalent bond between the structure and the small molecule, including but not limited to an alkyl group, carbonyl, amide, sulfide, ether, ester, arene, heteroarene, ketal, oxime, imine, enamine, alkene, alkyne, or other group.

A “small molecule moiety” refers to a small molecule that may form part of biomolecule or that may contain one or more FSK amino acid side chains represented by Formula (F). In embodiments, a small molecule moiety is a monovalent small molecule.

The term “pyrrolysyl-tRNA synthetase” refers to an enzyme (including homologs, isoforms, and functional fragments thereof) with pyrrolysyl-tRNA synthetase activity. Pyrrolysyl-tRNA synthetase is an aminoacyl-tRNA synthetase (aaRS) that catalyzes the reaction necessary to attach α-amino acid pyrrolysine to the cognate tRNA (tRNA^{pyl}), thereby allowing incorporation of pyrrolysine during proteinogenesis at amber stop codons (e.g., TAG). The term includes any recombinant or naturally-occurring form of pyrrolysyl-tRNA synthetase or variants, homologs, or isoforms thereof that maintain pyrrolysyl-tRNA synthetase activity (e.g. within at least 30%, 40%, 50%, 60%, 70%, 80%, 90%, 95%, or 100% activity compared to wild-type pyrrolysyl-tRNA synthetase). In aspects, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring pyrrolysyl-tRNA synthetase. In aspects, the pyrrolysyl-tRNA synthetase comprises the sequence set forth by SEQ ID NO:1. In aspects, the pyrrolysyl-tRNA synthetase is the sequence set forth by SEQ ID NO:1.

The term “mutant pyrrolysyl-tRNA synthetase” or “mutant PylRS” or “variant pyrrolysyl-tRNA synthetase” or “variant PylRS” refers to any pyrrolysyl-tRNA synthetase that has a different amino acid sequence from a wild-type amino acid sequence. In embodiments, the variant PylRS refers to any pyrrolysyl-tRNA synthetase that has a different amino acid sequence from a wild-type amino acid sequence of Methanomethylophilus alvus pyrrolysyl-tRNA synthetase set forth as SEQ ID NO:1. In aspects, “mutant pyrrolysyl-tRNA synthetase” refers to any pyrrolysyl-tRNA synthetase that catalyzes the attachment of fluorosulfonyloxybenzoyl-L-lysine (FSK) to a tRNA^{pyl}. In aspects, the mutant pyrrolysyl-tRNA synthetase includes SEQ ID NO:1 having mutations at one or more residues selected from the group consisting of tyrosine at position 126, methionine at position 129, valine at position 168, histidine at position 227, tyrosine at position 228, and lysine at position 229. In aspects, the mutant pyrrolysyl-tRNA synthetase includes SEQ ID NO:1 having the following five mutations: (i) Y126G; (ii) M129A; (iii) V168F; (iv) H227T, H227S, or H227I; and (v) Y228P. In aspects, the mutant pyrrolysyl-tRNA synthetase includes SEQ ID NO:1 having the following six mutations: (i) Y126G; (ii) M129A; (iii) V168F; (iv) H227T, H227S, or H227I; (v) Y228P; and (vi) L229V or L229I. In aspects, the mutant pyrrolysyl-tRNA synthetase includes SEQ ID NO:1 having mutations the following six mutations: Y126G; M129A; V168F; H227T; Y228P; and L229I. In aspects, the mutant pyrrolysyl-tRNA synthetase includes SEQ ID NO:1 having the following six mutations: Y126G; M129A; V168F; H227S; Y228P; and L229V. In aspects, the mutant pyrrolysyl-tRNA synthetase includes SEQ ID NO:1 having the following six mutations: Y126G; M129A; V168F; H227I; and Y228P. In aspects, the mutant pyrrolysyl-tRNA synthetase includes SEQ ID NO:1 having the following six mutations: Y126G; M129A; V168F; H227S; Y228P; and L229I. In aspects, the mutant pyrrolysyl-tRNA synthetase includes the sequence set forth by SEQ ID NO:2. In aspects, the mutant pyrrolysyl-tRNA synthetase is the sequence set forth by SEQ ID NO:2. In aspects, the mutant pyrrolysyl-tRNA synthetase is encoded by the sequence set forth by SEQ ID NO:2. In aspects, the mutant pyrrolysyl-tRNA synthetase further comprises six histidine residues at the N-terminus and/or the C-terminus. In aspects, the mutant pyrrolysyl-tRNA synthetase further comprises six histidine residues at the N-terminus. In aspects, the mutant pyrrolysyl-tRNA synthetase further comprises six histidine residues at the C-terminus. In aspects, the mutant pyrrolysyl-tRNA synthetase includes the sequence set forth by SEQ ID NO:86. In aspects, the mutant pyrrolysyl-tRNA synthetase is the sequence set forth by SEQ ID NO:86. In aspects, the mutant pyrrolysyl-tRNA synthetase is encoded by the sequence set forth by SEQ ID NO:86. In aspects, the mutant pyrrolysyl-tRNA synthetase includes the sequence set forth by SEQ ID NO:87. In aspects, the mutant pyrrolysyl-tRNA synthetase is the sequence set forth by SEQ ID NO:87. In aspects, the mutant pyrrolysyl-tRNA synthetase is encoded by the sequence set forth by SEQ ID NO:87. In aspects, “mutant pyrrolysyl-tRNA synthetase” is referred to as “pyrrolysyl-tRNA synthetase,” and the skilled artisan will readily recognize whether the pyrrolysyl-tRNA synthetase is mutant based on a comparison to the wild-type SEQ ID NO:1.

In embodiments, the mutant pyrrolysyl-tRNA synthetase includes SEQ ID NO:1 having 1 to 10 histidine residues at the C-terminus and/or the N-terminus (e.g., after the M residue); and having mutations at one or more residues selected from the group consisting of tyrosine at position 126, methionine at position 129, valine at position 168, histidine at position 227, tyrosine at position 228, and lysine at position 229. In aspects, the mutant pyrrolysyl-tRNA synthetase includes SEQ ID NO:1 having 1 to 10 histidine residues at the C-terminus and/or the N-terminus (e.g., after the M residue); and having the following five mutations: (i) Y126G; (ii) M129A; (iii) V168F; (iv) H227T, H227S, or H227I; and (v) Y228P. In aspects, the mutant pyrrolysyl-tRNA synthetase includes SEQ ID NO:1 having 1 to 10 histidine residues at the C-terminus and/or the N-terminus (e.g., after the M residue); and having the following six mutations: (i) Y126G; (ii) M129A; (iii) V168F; (iv) H227T, H227S, or H227I; (v) Y228P; and (vi) L229V or L229I. In aspects, the mutant pyrrolysyl-tRNA synthetase includes SEQ ID NO:1 having 1 to 10 histidine residues at the C-terminus and/or the N-terminus (e.g., after the M residue); and having mutations the following six mutations: Y126G; M129A; V168F; H227T; Y228P; and L229I. In aspects, the mutant pyrrolysyl-tRNA synthetase includes SEQ ID NO:1 having 6 histidine residues at the C-terminus; and having mutations the following six mutations: Y126G; M129A; V168F; H227T; Y228P; and L229I. In aspects, the mutant pyrrolysyl-tRNA synthetase includes SEQ ID NO:1 having 6 histidine residues at the N-terminus (after the M residue); and having mutations the following six mutations: Y126G; M129A; V168F; H227T; Y228P; and L229I. In aspects, the mutant pyrrolysyl-tRNA synthetase includes SEQ ID NO:1 having 1 to 10 histidine residues at the C-terminus and/or the N-terminus (e.g., after the M residue); and having the following six mutations: Y126G; M129A; V168F; H227S; Y228P; and L229V. In aspects, the mutant pyrrolysyl-tRNA synthetase includes SEQ ID NO:1 having 6 histidine residues at the C-terminus; and having the following six mutations: Y126G; M129A; V168F; H227S; Y228P; and L229V. In aspects, the mutant pyrrolysyl-tRNA synthetase includes SEQ ID NO:1 having 6 histidine residues at the N-terminus (after the M residue); and having the following six mutations: Y126G; M129A; V168F; H227S; Y228P; and L229V. In aspects, the mutant pyrrolysyl-tRNA synthetase includes SEQ ID NO:1 having 1 to 10 histidine residues at the C-terminus and/or the N-terminus (e.g., after the M residue); and having the following six mutations: Y126G; M129A; V168F; H227I; and Y228P. In aspects, the mutant pyrrolysyl-tRNA synthetase includes SEQ ID NO:1 having 6 histidine residues at the C-terminus; and having the following six mutations: Y126G; M129A; V168F; H227I; and Y228P. In aspects, the mutant pyrrolysyl-tRNA synthetase includes SEQ ID NO:1 having 6 histidine residues at the N-terminus (after the M residue); and having the following six mutations: Y126G; M129A; V168F; H227I; and Y228P. In aspects, the mutant pyrrolysyl-tRNA synthetase includes SEQ ID NO:1 having 1 to 10 histidine residues at the C-terminus and/or the N-terminus (e.g., after the M residue); and having the following six mutations: Y126G; M129A; V168F; H227S; Y228P; and L229I. In aspects, the mutant pyrrolysyl-tRNA synthetase includes SEQ ID NO:1 having 6 histidine residues at the C-terminus; and having the following six mutations: Y126G; M129A; V168F; H227S; Y228P; and L229I. In aspects, the mutant pyrrolysyl-tRNA synthetase includes SEQ ID NO:1 having 6 histidine residues at the N-terminus (after the M residue); and having the following six mutations: Y126G; M129A; V168F; H227S; Y228P; and L229I. In aspects, the mutant pyrrolysyl-tRNA synthetase includes the sequence set forth by SEQ ID NO:86. In aspects, the mutant pyrrolysyl-tRNA synthetase is the sequence set forth by SEQ ID NO:86. In aspects, the mutant pyrrolysyl-tRNA synthetase is encoded by the sequence set forth by SEQ ID NO:86. In aspects, the mutant pyrrolysyl-tRNA synthetase includes the sequence set forth by SEQ ID NO:87. In aspects, the mutant pyrrolysyl-tRNA synthetase is the sequence set forth by SEQ ID NO:87. In aspects, the mutant pyrrolysyl-tRNA synthetase is encoded by the sequence set forth by SEQ ID NO:87.

The term “tRNA^{Pyl}” refers to a single-stranded RNA molecule containing about 50 to about 100 nucleotides which fold via intrastrand base pairing to form a characteristic cloverleaf structure that carries a specific amino acid (e.g., pyrrolysine, FSK) and matches it to its corresponding codon (i.e., a complementary to the anticodon of the tRNA) on an mRNA during protein synthesis. The abbreviation “Pyl” of tRNA^{Pyl} stands for pyrrolysine. In embodiments, the anticodon comprises CUA, TTA, or TCA. In embodiments, the anticodon comprises CUA. In embodiments, the anticodon comprises TTA. In embodiments, the anticodon comprises TCA. In embodiments, the anticodon comprises at least one non-canonical base. Anticodon CUA is complementary to the amber stop codon. In aspects, tRNA^{Pyl} is attached to FSK. In aspects, tRNA^{Pyl} refers to a single-stranded RNA molecule containing about 50 to about 100 nucleotides. In aspects, tRNA^{Pyl} refers to a single-stranded RNA molecule containing about 60 to about 90 nucleotides. In aspects, tRNA^{Pyl} refers to a single-stranded RNA molecule containing about 65 to about 85 nucleotides. In aspects, tRNA^{Pyl} refers to a single-stranded RNA molecule containing about 70 to about 90 nucleotides. In aspects, tRNA^{Pyl} refers to a single-stranded RNA molecule containing about 60 to about 80 nucleotides.

The term “substrate-binding site” as used herein refers to residues located in the enzyme active site that form temporary bonds or interactions with the substrate. In aspects, the substrate-binding site of pyrrolysyl-tRNA synthetase refers to residues located in the active site of pyrrolysyl-tRNA synthetase that form temporary bonds or interactions with the amino acid substrate. In aspects, the substrate-binding site of pyrrolysyl-tRNA synthetase includes one or more of the following residues: tyrosine at position 126, methionine at position 129, valine at position 168, histidine at position 227, tyrosine at position 228, and lysine at position 229 as set forth in the amino acid sequence of SEQ ID NO:1.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The term “complex” refers to a composition that includes two or more components, where the components bind together to make a functional unit. In aspects, a complex described herein include a mutant pyrrolysyl-tRNA synthetase described herein and an amino acid substrate (e.g., FSK). In aspects, a complex described herein includes a mutant pyrrolysyl-tRNA synthetase described herein and a tRNA (e.g., tRNA^{pyl}). In aspects, a complex described herein includes a mutant pyrrolysyl-tRNA synthetase described herein, an amino acid substrate (e.g., FSK) and a tRNA (e.g., tRNA^{Pyl}). In aspects, a complex described herein includes at least two components selected from the group consisting of a mutant pyrrolysyl-tRNA synthetase described herein, an amino acid substrate (e.g., FSK), a polypeptide containing FSK, and a tRNA (e.g., tRNA^{Pyl})

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In aspects, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Contacting” is used in accordance with its plain ordinary meaning and refers to the process of allowing at least two distinct species (e.g. chemical compounds including biomolecules, biomolecule moieties, or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be biomolecules and/or biomolecule moieties as described herein. In aspects, contacting includes allowing two biomolecule moieties as described herein to interact, wherein the biomolecule moieties covalently bond to form a conjugate.

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH2, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In aspects, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982. In aspects, the first bioconjugate reactive group (e.g., maleimide moiety) is covalently attached to the second bioconjugate reactive group (e.g. a sulfhydryl). In aspects, the first bioconjugate reactive group (e.g., haloacetyl moiety) is covalently attached to the second bioconjugate reactive group (e.g. a sulfhydryl). In aspects, the first bioconjugate reactive group (e.g., pyridyl moiety) is covalently attached to the second bioconjugate reactive group (e.g. a sulfhydryl). In aspects, the first bioconjugate reactive group (e.g., —N-hydroxysuccinimide moiety) is covalently attached to the second bioconjugate reactive group (e.g. an amine). In aspects, the first bioconjugate reactive group (e.g., maleimide moiety) is covalently attached to the second bioconjugate reactive group (e.g. a sulfhydryl). In aspects, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc; (j) epoxides, which can react with, for example, amines and hydroxyl compounds; (k) phosphoramidites and other standard functional groups useful in nucleic acid synthesis; (1) metal silicon oxide bonding; (m) metal bonding to reactive phosphorus groups (e.g. phosphines) to form, for example, phosphate diester bonds; (n) azides coupled to alkynes using copper catalyzed cycloaddition click chemistry; (o) biotin conjugate can react with avidin or streptavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In aspects, the bioconjugate comprises a molecular entity derived from the reaction of an unsaturated bond, such as a maleimide, and a sulfhydryl group.

The term “in vitro translation system” refers to a system that provides for the in vitro synthesis of proteins in cell-free extracts that may provide for the identification of gene products (e.g., proteomics), localization of mutations through synthesis of truncated gene products, protein folding studies, and incorporation of modified or unnatural amino acids in to proteins. In embodiments, an in vitro translation system refers to a system that provides for the incorporation of modified or unnatural amino acids (e.g., FSK) into proteins. An exemplary in vitro translation system is PURExpress® In Vitro Protein Synthesis Kit by New England BioLabs, Inc. Exemplary components of an in vitro translation system include amino acids, wheat germ extract, cellular components for protein synthesis (e.g., tRNA, ribosomes, initiation factors, elongation factors, termination factors), salts (e.g., Mg²⁺, K⁺), and the like. In embodiments, the in vitro translation system is a rabbit reticulocyte system or a wheat germ extract system.

The terms “fluorosulfate-L-tyrosine” and “FSY” refer to the unnatural amino acid having the following structure:

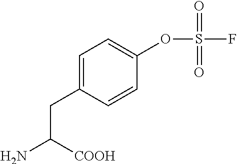 [SMILES: NC(Cc1ccc(OS(=O)(=O)F)cc1)C(=O)O]

or the stereoisomer thereof:

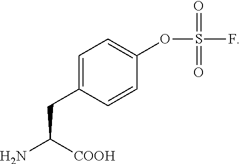 [SMILES: N[C@@H](Cc1ccc(OS(=O)(=O)F)cc1)C(=O)O]

FSY comprises the amino acid side chain of the formula:

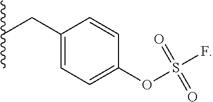 [SMILES: *Cc1ccc(OS(=O)(=O)F)cc1]

The terms “fluorosulfonyloxybenzoyl-L-lysine” and “FSK” refer to the unnatural amino acid having the structure of Formula (A):

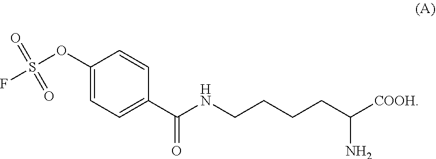 [SMILES: NC(CCCCNC(=O)c1ccc(OS(=O)(=O)F)cc1)C(=O)O]

which encompasses the stereoisomer thereof:

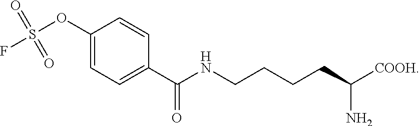 [SMILES: N[C@@H](CCCCNC(=O)c1ccc(OS(=O)(=O)F)cc1)C(=O)O]

FSK comprises the amino acid side chain of Formula (F):

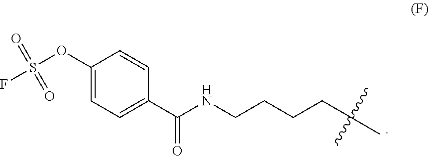 [SMILES: *CCCCNC(=O)c1ccc(OS(=O)(=O)F)cc1]

The term “FSK biomolecule” refers to a biomolecule comprising the FSK unnatural amino acid and/or the amino acid side chain thereof.

The term “biomolecule conjugate” or “FSK biomolecule conjugate” refers to any biomolecule comprising a bioconjugate linker (“FSK bioconjugate linker”) having the structure of Formula (D):

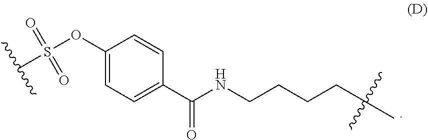 [SMILES: *CCCCNC(=O)c1ccc(OS(*)(=O)=O)cc1]

The term “FSK protein” refers to a protein comprising the FSK unnatural amino acid and/or the amino acid side chain thereof.

The term “protein conjugate” or “FSK protein conjugate” refers to any protein comprising a bioconjugate linker having the structure of Formula (D):

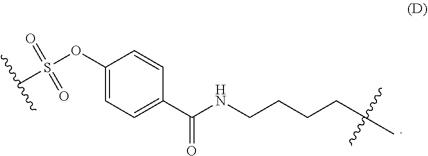 [SMILES: *CCCCNC(=O)c1ccc(OS(*)(=O)=O)cc1]

The term “sulfur-fluoride exchange reaction” or “SuFEx” refers to a type of click chemistry as described in detail by, e.g., Dong et al, Angewandte Chemie, 53(36):9340-9448 (2014); Wang et al, J. Am. Chem. Soc., 140(15):4995-4999 (2018); and as described in the examples herein. The term “proximally-enabled” SuFEx refers to the sulfur-fluoride exchange reaction occurring when the reactive species are proximal to each other, i.e., spatially close enough for the SuFEx reaction to occur. The proximity may occur within a single biomolecule (e.g., protein) or between two different biomolecules (e.g., proteins). The skilled artisan could readily determine whether the reactive species are sufficiently proximal for the reaction to occur (e.g., sulfur-fluoride exchange reaction between FSK and lysine, histidine, or tyrosine to form the bioconjugate, the moiety of Formula (A), (B), or (C), or the protein of Formula (I), (II), or (III)).

The term “intermolecular linker” refers to a linking group between two different biomolecules. For example, when the compound of Formula (E), (I), (II), or (III) has an intermolecular linker, then the peptidyl moiety of R¹ is a first protein and the peptidyl moiety of R² is a second protein, such that the first protein and the second protein are covalently bonded via the moiety of Formula (E) (I), (II), or (III). In aspects, the first protein and the second protein are different proteins, e.g., providing an intermolecular linker between two different proteins, such as a single-domain antibody and a membrane receptor.

The term “intramolecular linker” refers to a linking group within a single biomolecule. For example, when the compound of Formula (E) (I), (II), or (III) has an intramolecular linker, then the peptidyl moiety of R¹ and the peptidyl moiety of R² are in the same protein. In aspects, the first protein and the second protein are the same protein, i.e., providing an intermolecular linker within a single protein.

### Biomolecules and Biomolecule Conjugates

Provided herein are biomolecules and biomolecule conjugates formed through the interaction of latent bioreactive unnatural amino acids with naturally occurring amino acids. Fluorosulfonyloxybenzoyl-L-lysine (FSK or N6-(4-((fluorosulfonyl)oxy)benzoyl)-L-lysine), a latent bioreactive unnatural amino acid, facilitates formation of covalent bonds with proximal target amino acid residues (e.g., lysine, histidine, tyrosine) by undergoing a click chemistry reaction (e.g., sulfur-fluoride exchange reaction (SuFEx)). For example, FSK may be inserted into or replace an amino acid in a naturally occurring protein, thereby endowing the protein with the ability to form a covalent bond with proximally positioned target amino acid residues (e.g., lysine, histidine, tyrosine) on the protein itself or with proteins it naturally interacts with. FSK may be used to facilitate the formation of covalent bonds between or within proteins in both in vitro and in vivo conditions, owing, at least in part, to its being non-toxic to cells. As such, the latent bioreactive unnatural amino acid FSK is useful for covalently linking biomolecules (e.g., proteins, carbohydrates, nucleic acids) to form biomolecule conjugates. In aspects, the latent bioreactive unnatural amino acid FSK is useful for covalently linking biomolecule moieties (e.g., peptidyl moieties) within a single biomolecule (e.g., protein). In aspects, the latent bioreactive unnatural amino acid FSK is useful for covalently linking biomolecule moieties (e.g., peptidyl moieties) in different biomolecules (e.g., covalently linking two proteins). In aspects, the latent bioreactive unnatural amino acid FSK is useful for covalently linking single domain antibodies to membrane receptors.

As shown herein, FSK, as a latent bioreactive unnatural amino acid, has shown excellent chemical functionality (i.e., superior properties) compared to previously described bioreactive unnatural amino acids. For example, FSK is stable, nontoxic and nonreactive inside cells, yet when placed in proximity to target residues it becomes reactive under cellular conditions. FSK is able to react with lysine, histidine, and tyrosine specifically with great selectivity via proximity-enabled SuFEx reaction within and between proteins under physiological conditions.

Provided herein are biomolecules comprising one or more latent bioreactive unnatural amino acids. In aspects, the biomolecule is a protein, a nucleic acid, or a carbohydrate. In aspects, the biomolecule is a protein. In aspects, FSK and the lysine, histidine, or tyrosine are in an α-strand of the protein. In aspects, FSK and the lysine, histidine, or tyrosine are in a β-strand of the protein. In aspects, the protein is a single-domain antibody. In aspects, the protein is a membrane receptor. In aspects, the latent bioreactive unnatural amino acid is fluorosulfonyloxybenzoyl-L-lysine (FSK) having the structure of Formula (A):

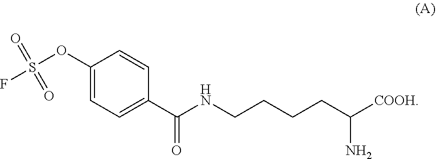 [SMILES: NC(CCCCNC(=O)c1ccc(OS(=O)(=O)F)cc1)C(=O)O]

In aspects, the biomolecule is a protein comprising the FSK unnatural amino acid. In aspects, the protein comprises at least one FSK. In embodiments, the protein comprises one FSK. In aspects, the proteins comprises two or more FSK. In aspects, the proteins comprises two FSK. In aspects, the proteins comprises three FSK. In aspects, the biomolecule is a protein comprising the FSK amino acid side chain represented by Formula (F):

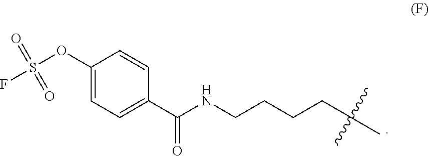 [SMILES: *CCCCNC(=O)c1ccc(OS(=O)(=O)F)cc1]

In aspects, the protein comprises FSK that is proximal to lysine, histidine, tyrosine, or a combination of two or more thereof. In aspects, the protein comprises FSK that is proximal to lysine. In aspects, the protein comprises FSK that is proximal to histidine. In aspects, the protein comprises FSK that is proximal to tyrosine. In aspects, the protein is an antibody or an antibody variant. In aspects, the protein is an antibody, an antigen-binding fragment, a single-chain variable fragment, a single-domain antibody, or an affibody.

“Proximal” means that FSK and lysine, histidine, or tyrosine are close enough to each other for a SuFEx reaction to successfully occur. In aspects, “proximal” means that FSK is within 1 to 50 amino acids of a lysine, histidine, or tyrosine. In aspects “proximal” means that FSK is within 1 to 45 amino acids of a lysine, histidine, or tyrosine. In aspects “proximal” means that FSK is within 1 to 40 amino acids of a lysine, histidine, or tyrosine. In aspects “proximal” means that FSK is within 1 to 35 amino acids of a lysine, histidine, or tyrosine. In aspects “proximal” means that FSK is within 1 to 30 amino acids of a lysine, histidine, or tyrosine. In aspects “proximal” means that FSK is within 1 to 25 amino acids of a lysine, histidine, or tyrosine. In aspects “proximal” means that FSK is within 1 to 20 amino acids of a lysine, histidine, or tyrosine. In aspects “proximal” means that FSK is within 1 to 15 amino acids of a lysine, histidine, or tyrosine. In aspects “proximal” means that FSK is within 1 to 10 amino acids of a lysine, histidine, or tyrosine. In aspects “proximal” means that FSK is within 1 to 9 amino acids of a lysine, histidine, or tyrosine. In aspects “proximal” means that FSK is within 1 to 8 amino acids of a lysine, histidine, or tyrosine. In aspects “proximal” means that FSK is within 1 to 7 amino acids of a lysine, histidine, or tyrosine. In aspects “proximal” means that FSK is within 1 to 6 amino acids of a lysine, histidine, or tyrosine. In aspects “proximal” means that FSK is within 1 to 5 amino acids of a lysine, histidine, or tyrosine. In aspects “proximal” means that FSK is within 1 to 4 amino acids of a lysine, histidine, or tyrosine. In aspects “proximal” means that FSK is within 1 to 3 amino acids of a lysine, histidine, or tyrosine. In aspects “proximal” means that FSK is within 1 to 2 amino acids of a lysine, histidine, or tyrosine. In aspects “proximal” means that FSK is adjacent a lysine, histidine, or tyrosine.

Provided here are biomolecule conjugates comprising a first biomolecule moiety conjugated to a second biomolecule moiety through a bioconjugate linker, wherein the bioconjugate linker has the structure of Formula (D):

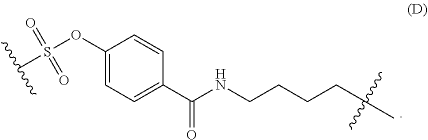 [SMILES: *CCCCNC(=O)c1ccc(OS(*)(=O)=O)cc1]

In aspects, the first biomolecule moiety and the second biomolecule moiety are each independently a peptidyl moiety. In aspects, the biomolecule conjugate is a protein conjugate. In aspects, the biomolecule conjugate is a protein conjugate, wherein the bioconjugate linker is an intramolecular linker. In aspects, the protein conjugate comprises a plurality of intramolecular linkers. In aspects, the biomolecule conjugate is a protein conjugate, wherein the bioconjugate linker is an intermolecular linker. In aspects, the protein conjugate comprises a plurality of intermolecular linkers. In aspects, the protein conjugate comprises intramolecular linkers and intermolecular linkers.

In embodiments, the biomolecule conjugate has the structure of Formula (E):

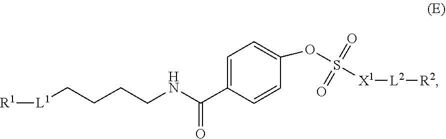

alternatively described as having the formula:

R¹-L¹-A-X¹-L²-R²;

wherein A is the bioconjugate linker of Formula (D); R¹ is the first biomolecule moiety; R² is the second bioconjugate moiety; L¹ is a bond or a first covalent linker; L² is a bond of a second covalent linker; and X¹ is —NR′—, —O—, —S—, or

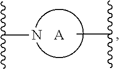

wherein ring A is a substituted or unsubstituted heteroarylene or substituted or unsubstituted heterocycloalkylene, and wherein the nitrogen in A is attached to the bioconjugate linker. R⁵ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L¹ is a bond, —S(O)₂—, —NR^{3A}—, —O—, —S—, —C(O)—, —C(O)NR^{3A}—, —NR^{3A}C(O)—, —NR^{3A}C(O)NR^{3B}—, —C(O)—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R^{3A} and R^{3B} are independently hydrogen, substituted or unsubstituted alkylyl, substituted or unsubstituted heteroalkylyl, substituted or unsubstituted cycloalkylyl, substituted or unsubstituted heterocycloalkylyl, substituted or unsubstituted arylyl, or substituted or unsubstituted heteroarylyl.

L² is a bond, —S(O)₂—, —NR^{4A}—, —O—, —S—, —C(O)—, —C(O)NR^{4A}—, —NR^{4A}C(O)—, —NR^{4A}C(O)NR^{4B}—, —C(O)—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or substituted or unsubstituted alkylarylene. In embodiments, L² is a bond, —S(O)₂—, —NR^{4A}—, —O—, —S—, —C(O)—, —C(O)NR^{4A}—, —NR^{4A}C(O)—, —NR^{4A}C(O)NR^{4B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene. R^{4A} and R^{4B} are independently hydrogen, substituted or unsubstituted alkylyl, substituted or unsubstituted heteroalkylyl, substituted or unsubstituted cycloalkylyl, substituted or unsubstituted heterocycloalkylyl, substituted or unsubstituted arylyl, or substituted or unsubstituted heteroarylyl.

X¹ is —NR⁵—, —O—, —S—, or

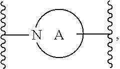

wherein ring A is a substituted or unsubstituted heteroarylene or substituted or unsubstituted heterocycloalkylene. In aspects, X¹ is —NR⁵—. In aspects X¹ is —O—. In aspects, X is —S—. In aspects, X¹ is

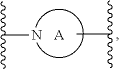

wherein ring A is a substituted or unsubstituted heteroarylene or substituted or unsubstituted heterocycloalkylene. In aspects, ring A is substituted or unsubstituted heteroarylene. In aspects, ring A is substituted or unsubstituted heterocycloalkylene. In aspects, ring A is unsubstituted heteroarylene. In aspects, ring A is unsubstituted heterocycloalkylene. In aspects, ring A is substituted heterocycloalkylene (e.g., 3 to 8 membered, 3 to 6 membered, 4 to 6 membered, 4 to 5 membered, or 5 to 6 membered). In aspects, ring A is unsubstituted heterocycloalkylene (e.g., 3 to 8 membered, 3 to 6 membered, 4 to 6 membered, 4 to 5 membered, or 5 to 6 membered). In aspects, ring A is substituted or unsubstituted heteroarylene (e.g., 5 to 10 membered, 5 to 9 membered, or 5 to 6 membered). In aspects, ring A is substituted heteroarylene (e.g., 5 to 10 membered, 5 to 9 membered, or 5 to 6 membered). In aspects, ring A is unsubstituted heteroarylene (e.g., 5 to 10 membered, 5 to 9 membered, or 5 to 6 membered). In embodiments, X¹ is a bond.

In embodiments, R⁵ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In aspects, R⁵ is hydrogen.

In embodiments, R⁵ is hydrogen, substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R⁵ is hydrogen, substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₈, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₈, C₆-C₅) aryl or substituted or unsubstituted (e.g., 5 to 10 membered, 5 to 8 membered, 5 to 6 membered) heteroaryl.

In embodiments, R⁵ is hydrogen, unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₈, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₈, C₆-C₅) aryl or unsubstituted (e.g., 5 to 10 membered, 5 to 8 membered, 5 to 6 membered) heteroaryl.

In embodiments, L¹ is a bond, —S(O)₂—, —NR^{3A}—, —O—, —S—, —C(O)—, —C(O)NR^{3A}—, —NR^{3A}C(O)—, —NR^{3A}C(O)NR^{3B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹ is a bond, —S(O)₂—, —NR^{3A}—, —O—, —S—, —C(O)—, —C(O)NR^{3A}—, —NR^{3A}C(O)—, —NR^{3A}C(O)NR^{3B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In aspects, L¹ is a bond, substituted or unsubstituted alkylene, or substituted or unsubstituted heteroalkylene. In aspects, L¹ is a bond, unsubstituted alkylene, or unsubstituted heteroalkylene. In aspects, L¹ is unsubstituted alkylene. In aspects, L¹ is unsubstituted heteroalkylene. In aspects, L¹ is a bond.

In embodiments, L¹ is —O—, —S—, R³²-substituted or unsubstituted C₁-C₂ alkylene (e.g., C₁ or C₂) or R³²— substituted or unsubstituted 2 membered heteroalkylene. In aspects, L¹ is R³²-substituted or unsubstituted alkylene (e.g., C₁-C₈alkylene, C₁-C₆ alkylene, or C₁-C₄ alkylene), R³²-substituted or unsubstituted heteroalkylene (e.g., 2 to 8 membered heteroalkylene, 2 to 6 membered heteroalkylene, or 2 to 4 membered heteroalkylene), R³²-substituted or unsubstituted cycloalkylene (e.g., C₃-C₈ cycloalkylene, C₃-C₆ cycloalkylene, or C₅-C₆ cycloalkylene), R³²-substituted or unsubstituted heterocycloalkylene (e.g., 3 to 8 membered heterocycloalkylene, 3 to 6 membered heterocycloalkylene, or 5 to 6 membered heterocycloalkylene), R³²-substituted or unsubstituted arylene (e.g., C₆-C₁₀ arylene, C₁₀ arylene, or phenylene), or R³²-substituted or unsubstituted heteroarylene (e.g., 5 to 10 membered heteroarylene, 5 to 9 membered heteroarylene, or 5 to 6 membered heteroarylene). In aspects, L¹ is independently —O—, —S—, unsubstituted C₁-C₂ alkylene (e.g., C₁ or C₂) or unsubstituted 2 membered heteroalkylene. In aspects, L¹ is independently unsubstituted methylene. In aspects, L¹ is independently unsubstituted ethylene. In aspects, L¹ is substituted 2 membered heteroalkylene. In aspects, L¹ is substituted 3 membered heteroalkylene. In aspects, L¹ is substituted 4 membered heteroalkylene. In aspects, L¹ is an unsubstituted 2 membered heteroalkylene. In aspects, L¹ is an unsubstituted 3 membered heteroalkylene. In aspects, L¹ is an unsubstituted 4 membered heteroalkylene.

R³² is independently oxo, halogen, —CX³²₃, —CHX³²₂, —CH₂X³², —OCX³²₃, —OCH₂X³², —OCHX³²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³³-substituted or unsubstituted alkyl (e.g., C₁-C₈, C₁-C₆, C₁-C₄, or C₁-C₂), R³³-substituted or unsubstituted heteroalkyl (e.g., 2 to 8 membered, 2 to 6 membered, 4 to 6 membered, 2 to 3 membered, or 4 to 5 membered), R³³-substituted or unsubstituted cycloalkyl (e.g., C₃-C₈, C₃-C₆, C₄-C₆, or C₅-C₆), R³³-substituted or unsubstituted heterocycloalkyl (e.g., 3 to 8 membered, 3 to 6 membered, 4 to 6 membered, 4 to 5 membered, or 5 to 6 membered), R³³-substituted or unsubstituted aryl (e.g., C₆-C₁₀ or phenyl), or R³³-substituted or unsubstituted heteroaryl (e.g., 5 to 10 membered, 5 to 9 membered, or 5 to 6 membered). In aspects, R³² is independently oxo, halogen, —CX³²₃, —CHX³²₂, —CH₂X³², —OCX³²₃, —OCH₂X³², —OCHX³²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³² is independently —F, —Cl, —Br, or —I.

In embodiments, R³² is independently unsubstituted methyl. In aspects, R³² is independently unsubstituted ethyl.

R³³ is independently oxo, halogen, —CX³³₃, —CHX³³₂, —CH₂X³³, —OCX³³₃, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³⁴-substituted or unsubstituted alkyl (e.g., C₁-C₈, C₁-C₆, C₁-C₄, or C₁-C₂), R³⁴-substituted or unsubstituted heteroalkyl (e.g., 2 to 8 membered, 2 to 6 membered, 4 to 6 membered, 2 to 3 membered, or 4 to 5 membered), R³⁴-substituted or unsubstituted cycloalkyl (e.g., C₃-C₈, C₃-C₆, C₄-C₆, or C₅-C₆), R³⁴-substituted or unsubstituted heterocycloalkyl (e.g., 3 to 8 membered, 3 to 6 membered, 4 to 6 membered, 4 to 5 membered, or 5 to 6 membered), R³⁴-substituted or unsubstituted aryl (e.g., C₆-C₁₀ or phenyl), or R³⁴-substituted or unsubstituted heteroaryl (e.g., 5 to 10 membered, 5 to 9 membered, or 5 to 6 membered). In aspects, R³³ is independently oxo, halogen, —CX³³₃, —CHX³³₂, —CH₂X³³, —OCX³³₃, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³³ is independently —F, —Cl, —Br, or —I.

In embodiments, R³³ is independently unsubstituted methyl. In aspects, R³³ is independently unsubstituted ethyl.

R³⁴ is independently oxo, halogen, —CX³⁴₃, —CHX³⁴₂, —CH₂X³⁴, —OCX³⁴₃, —OCH₂X³⁴, —OCHX³⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁴ is independently —F, —Cl, —Br, or —I.

In embodiments, R³⁴ is independently unsubstituted methyl. In aspects, R³⁴ is independently unsubstituted ethyl.

In embodiments, R^{3A} is hydrogen, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, R^{3A} is hydrogen, substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R^{3A} is hydrogen, substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₈, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₈, C₆-C₅) aryl or substituted or unsubstituted (e.g., 5 to 10 membered, 5 to 8 membered, 5 to 6 membered) heteroaryl.

In embodiments, R^{3A} is hydrogen, unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₈, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₈, C₆-C₅) aryl or unsubstituted (e.g., 5 to 10 membered, 5 to 8 membered, 5 to 6 membered) heteroaryl.

In embodiments, R^{3B} is hydrogen, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, R^{3B} is hydrogen, substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R^{3B} is hydrogen, substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₈, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₈, C₆-C₅) aryl or substituted or unsubstituted (e.g., 5 to 10 membered, 5 to 8 membered, 5 to 6 membered) heteroaryl.

In embodiments, R^{3B} is hydrogen, unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₈, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₈, C₆-C₅) aryl or unsubstituted (e.g., 5 to 10 membered, 5 to 8 membered, 5 to 6 membered) heteroaryl.

In embodiments, L² is a bond, —S(O)₂—, —NR^{4A}—, —O—, —S—, —C(O)—, —C(O)NR^{4A}—, —NR^{4A}C(O)—, —NR^{4A}C(O)NR^{4B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or substituted or unsubstituted alkylarylene. In embodiments, L² is a bond, —S(O)₂—, —NR^{4A}—, —O—, —S—, —C(O)—, —C(O)NR^{4A}—, —NR^{4A}C(O)—, —NR^{4A}C(O)NR^{4B}—, —C(O)—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L² is a bond, substituted or unsubstituted alkylene, substituted or unsubstituted heteroalkylene, or substituted or unsubstituted alkylarylene. In embodiments, L² is a bond, substituted or unsubstituted alkylene, or substituted or unsubstituted heteroalkylene. In aspects, L² is a bond, unsubstituted alkylene, or unsubstituted heteroalkylene. In aspects, L² is unsubstituted alkylene. In aspects, L² is unsubstituted heteroalkylene. In aspects, L² is a bond. In aspects, L² is a bond, or substituted or unsubstituted alkylarylene. In aspects, L² is a bond or unsubstituted alkylarylene. In aspects, L² is unsubstituted alkylarylene. In aspects, L² is benzylene.

In embodiments, L² is —O—, —S—, R³⁵-substituted or unsubstituted C₁-C₂ alkylene (e.g., C₁ or C₂) or R³⁵— substituted or unsubstituted 2 membered heteroalkylene. In aspects, L² is R³⁵-substituted or unsubstituted alkylene (e.g., C₁-C₈alkylene, C₁-C₆ alkylene, or C₁-C₄ alkylene), R³⁵-substituted or unsubstituted heteroalkylene (e.g., 2 to 8 membered heteroalkylene, 2 to 6 membered heteroalkylene, or 2 to 4 membered heteroalkylene), R³⁵-substituted or unsubstituted cycloalkylene (e.g., C₃-C₈ cycloalkylene, C₃-C₆ cycloalkylene, or C₅-C₆ cycloalkylene), R³⁵-substituted or unsubstituted heterocycloalkylene (e.g., 3 to 8 membered heterocycloalkylene, 3 to 6 membered heterocycloalkylene, or 5 to 6 membered heterocycloalkylene), R³⁵-substituted or unsubstituted arylene (e.g., C₆-C₁₀ arylene, C₁₀ arylene, or phenylene), or R³⁵-substituted or unsubstituted heteroarylene (e.g., 5 to 10 membered heteroarylene, 5 to 9 membered heteroarylene, or 5 to 6 membered heteroarylene). In aspects, L² is —O—, —S—, unsubstituted C₁-C₂ alkylene (e.g., C₁ or C₂) or unsubstituted 2 membered heteroalkylene. In aspects, L² is unsubstituted methylene. In aspects, L² is unsubstituted ethylene. In aspects, L² is substituted 2 membered heteroalkylene. In aspects, L² is substituted 3 membered heteroalkylene. In aspects, L² is substituted 4 membered heteroalkylene. In aspects, L² is an unsubstituted 2 membered heteroalkylene. In aspects, L² is an unsubstituted 3 membered heteroalkylene. In aspects, L² is an unsubstituted 4 membered heteroalkylene.

R³⁵ is independently oxo, halogen, —CX³⁵₃, —CHX³⁵₂, —CH₂X³⁵, —OCX³⁵₃, —OCH₂X³⁵, —OCHX³⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³⁶-substituted or unsubstituted alkyl (e.g., C₁-C₈, C₁-C₆, C₁-C₄, or C₁-C₂), R³⁶-substituted or unsubstituted heteroalkyl (e.g., 2 to 8 membered, 2 to 6 membered, 4 to 6 membered, 2 to 3 membered, or 4 to 5 membered), R³⁶-substituted or unsubstituted cycloalkyl (e.g., C₃-C₈, C₃-C₆, C₄-C₆, or C₅-C₆), R³⁶-substituted or unsubstituted heterocycloalkyl (e.g., 3 to 8 membered, 3 to 6 membered, 4 to 6 membered, 4 to 5 membered, or 5 to 6 membered), R³⁶-substituted or unsubstituted aryl (e.g., C₆-C₁₀ or phenyl), or R³⁶-substituted or unsubstituted heteroaryl (e.g., 5 to 10 membered, 5 to 9 membered, or 5 to 6 membered). In aspects, R³⁵ is independently oxo, halogen, —CX³⁵₃, —CHX³⁵₂, —CH₂X³⁵, —OCX³⁵₃, —OCH₂X³⁵, —OCHX³⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁵ is independently —F, —Cl, —Br, or —I.

In embodiments, R³⁵ is independently unsubstituted methyl. In aspects, R³⁵ is independently unsubstituted ethyl.

R³⁶ is independently oxo, halogen, —CX³⁶₃, —CHX³⁶₂, —CH₂X³⁶, —OCX³⁶₃, —OCH₂X³⁶, —OCHX³⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³⁷-substituted or unsubstituted alkyl (e.g., C₁-C₈, C₁-C₆, C₁-C₄, or C₁-C₂), R³⁷-substituted or unsubstituted heteroalkyl (e.g., 2 to 8 membered, 2 to 6 membered, 4 to 6 membered, 2 to 3 membered, or 4 to 5 membered), R³⁷-substituted or unsubstituted cycloalkyl (e.g., C₃-C₈, C₃-C₆, C₄-C₆, or C₅-C₆), R³⁷-substituted or unsubstituted heterocycloalkyl (e.g., 3 to 8 membered, 3 to 6 membered, 4 to 6 membered, 4 to 5 membered, or 5 to 6 membered), R³⁷-substituted or unsubstituted aryl (e.g., C₆-C₁₀ or phenyl), or R³⁷-substituted or unsubstituted heteroaryl (e.g., 5 to 10 membered, 5 to 9 membered, or 5 to 6 membered). In aspects, R³⁶ is independently oxo, halogen, —CX³⁶₃, —CHX³⁶₂, —CH₂X³⁶, —OCX³⁶₃, —OCH₂X³⁶, —OCHX³⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁶ is independently —F, —Cl, —Br, or —I.

In embodiments, R³⁶ is independently unsubstituted methyl. In aspects, R³⁶ is independently unsubstituted ethyl.

R³⁷ is independently oxo, halogen, —CX³⁷₃, —CHX³⁷₂, —CH₂X³⁷, —OCX³⁷₃, —OCH₂X³⁷, —OCHX³⁷₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁷ is independently —F, —Cl, —Br, or —I.

In embodiments, R³⁷ is independently unsubstituted methyl. In aspects, R³⁷ is independently unsubstituted ethyl.

In embodiments, R^{4A} is hydrogen, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, R^{4A} is hydrogen, substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R^{4A} is hydrogen, substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₈, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₈, C₆-C₅) aryl or substituted or unsubstituted (e.g., 5 to 10 membered, 5 to 8 membered, 5 to 6 membered) heteroaryl.

In embodiments, R^{4A} is hydrogen, unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₈, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₈, C₆-C₅) aryl or unsubstituted (e.g., 5 to 10 membered, 5 to 8 membered, 5 to 6 membered) heteroaryl.

In embodiments, R^{4B} is hydrogen, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, R^{4B} is hydrogen, substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R^{4B} is hydrogen, substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₈, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₈, C₆-C₅) aryl or substituted or unsubstituted (e.g., 5 to 10 membered, 5 to 8 membered, 5 to 6 membered) heteroaryl.

In embodiments, R^{4B} is hydrogen, unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₈, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₈, C₆-C₅) aryl or unsubstituted (e.g., 5 to 10 membered, 5 to 8 membered, 5 to 6 membered) heteroaryl.

In embodiments, X¹ is imidazolylene, —NH— or —O—. In aspects, X¹ is imidazolylene (i.e., a divalent imidazole). In aspects, X¹ is —NH—. In aspects, X¹ is —O—.

In embodiments, the first biomolecule moiety is a peptidyl moiety. In aspects, the second biomolecule moiety is a peptidyl moiety. In aspects, the first biomolecule moiety is a peptidyl moiety and the second biomolecule moiety is a peptidyl moiety. In aspects, the peptidyl moieties in the first biomolecule moiety and the second biomolecule moiety are in the same protein. In aspects, the peptidyl moieties in the first biomolecule moiety and the second biomolecule moiety are in different proteins. In embodiments, the different proteins are a single-domain antibody and a membrane receptor. In embodiments, the different proteins are an antibody and a membrane receptor. In embodiments, the different proteins are an antigen-binding fragment and a membrane receptor. In embodiments, the different proteins are an affibody and a membrane receptor. In embodiments, the different proteins are a single-chain variable fragment and a membrane receptor.

In embodiments, -L¹-R¹ is a peptidyl moiety. In embodiments, -L²-R² is a peptidyl moiety. In aspects, the peptidyl moieties of -L¹-R¹ and -L²-R² are in the same protein. In aspects, the peptidyl moieties of -L¹-R¹ and -L²-R² are in different proteins. In aspects, L¹ is a bond. In aspects, L² is a bond. In aspects, L¹ and L² are a bond. In embodiments, the different proteins are a single-domain antibody and a membrane receptor. In embodiments, the different proteins are an antibody and a membrane receptor. In embodiments, the different proteins are a single-chain variable fragment and a membrane receptor. In embodiments, the different proteins are an affibody and a membrane receptor. In embodiments, the different proteins are an antigen-binding fragment and a membrane receptor.

In embodiments, the first biomolecule moiety is a nucleic acid moiety or a carbohydrate moiety. In embodiments, the first biomolecule moiety is a nucleic acid moiety. In embodiments, the first biomolecule moiety is a carbohydrate moiety. In embodiments, the second biomolecule moiety is a nucleic acid moiety or a carbohydrate moiety. In embodiments, the second biomolecule moiety is a nucleic acid moiety. In embodiments, the second biomolecule moiety is a carbohydrate moiety.

In embodiments, -L¹-R¹ is a nucleic acid moiety or a carbohydrate moiety. In aspects, -L¹-R¹ is a nucleic acid moiety. In aspects, -L¹-R¹ is a carbohydrate moiety. In aspects, -L²-R² is a nucleic acid moiety or a carbohydrate moiety. In aspects, -L²-R² is a nucleic acid moiety. In aspects, -L²-R² is a carbohydrate moiety. In aspects, L¹ is a bond. In aspects, L² is a bond. In aspects, L¹ and L² are a bond.

In embodiments, the first biomolecule moiety is selected from the group consisting of a small molecule moiety, peptidyl moiety, a nucleic acid moiety, and a carbohydrate moiety. In aspects, the second biomolecule moiety is selected from the group consisting of a small molecule moiety, a peptidyl moiety, a nucleic acid moiety, and a carbohydrate moiety. In aspects, the first biomolecule moiety is same as the second biomolecule moiety. In aspects, the first biomolecule moiety is different from the second biomolecule moiety. In aspects, the first biomolecule moiety and the second biomolecule moiety are within the same biomolecule. In aspects, the first biomolecule moiety and the second biomolecule moiety are in different biomolecules. In aspects, the first biomolecule moiety is a small molecule moiety and the second biomolecule moiety is a peptidyl moiety. In aspects, the first biomolecule moiety is a peptidyl moiety and the second biomolecule moiety is a small molecule moiety.

In embodiments, the first biomolecule moiety is selected from the group consisting of a peptidyl moiety, a nucleic acid moiety, and a carbohydrate moiety. In aspects, the second biomolecule moiety is selected from the group consisting of a peptidyl moiety, a nucleic acid moiety, and a carbohydrate moiety. In aspects, the first biomolecule moiety is same as the second biomolecule moiety. In aspects, the first biomolecule moiety is different from the second biomolecule moiety. In aspects, the first biomolecule moiety and the second biomolecule moiety are within the same biomolecule. In aspects, the first biomolecule moiety and the second biomolecule moiety are in different biomolecules. In aspects, the first biomolecule moiety and the second biomolecule moiety are each independently a peptidyl moiety.

In embodiments, -L¹-R¹ is selected from the group consisting of a small molecule moiety, a peptidyl moiety, a nucleic acid moiety and a carbohydrate moiety. In aspects, -L²-R² is selected from the group consisting of a small molecule moiety, a peptidyl moiety, a nucleic acid moiety and a carbohydrate moiety. In aspects, -L¹-R¹ is a small molecule moiety. In aspects,-L²-R² is a small molecule moiety. In aspects, L¹ is a bond. In aspects, L² is a bond. In aspects, L¹ and L² are a bond.

In embodiments, -L¹-R¹ is selected from the group consisting of a peptidyl moiety, a nucleic acid moiety and a carbohydrate moiety. In aspects, -L²-R² is selected from the group consisting of a peptidyl moiety, a nucleic acid moiety and a carbohydrate moiety. In aspects, -L¹-R¹ is the same as -L²-R². In aspects, -L¹-R¹ is different from -L²-R². In aspects, -L¹-R¹ and -L²-R² are each independently a peptidyl moiety. In aspects, L¹ is a bond. In aspects, L² is a bond. In aspects, L¹ and L² are a bond.

In aspects, the disclosure provides a protein comprising a moiety of Formula (IV), a moiety of Formula (V), a moiety of Formula (VI), or a combination of two or more thereof:

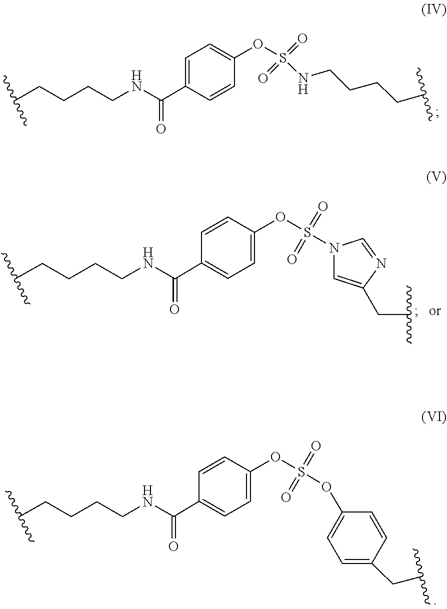 [SMILES: *CCCCNC(=O)c1ccc(OS(=O)(=O)NCCCC*)cc1 ; *CCCCNC(=O)c1ccc(OS(=O)(=O)n2cnc(C*)c2)cc1 ; *CCCCNC(=O)c1ccc(OS(=O)(=O)Oc2ccc(C*)cc2)cc1]

In aspects, the protein comprises a moiety of Formula (IV). In aspects, the protein comprises a moiety of Formula (V). In aspects, the protein comprises a moiety of Formula (VI). In aspects, the protein comprises a moiety of Formula (IV) and a moiety of Formula (V). In aspects, the protein comprises a moiety of Formula (IV) and a moiety of Formula (VI). In aspects, the protein comprises a moiety of Formula (V) and a moiety of Formula (VI). In aspects, the protein comprises a moiety of Formula (IV), a moiety of Formula (V), and a moiety of Formula (VI). In aspect, the moieties of Formula (IV), (V), (VI), or a combination thereof, form intramolecular covalent bonds. In aspect, the moiety of Formula (IV) forms an intramolecular covalent bond. In aspect, the moiety of Formula (V) forms an intramolecular covalent bond. In aspect, the moiety of Formula (VI) forms an intramolecular covalent bond. In aspect, the moieties of Formula (IV) and (V) form intramolecular covalent bonds. In aspect, the moieties of Formula (IV) and (VI) form intramolecular covalent bonds. In aspect, the moieties of Formula (V) and (VI) form intramolecular covalent bonds. In aspect, the moieties of Formula (IV), (V), and (VI) form intramolecular covalent bonds. In aspect, the moieties of Formula (IV), (V), (VI), or a combination thereof form intermolecular covalent bonds. In aspect, the moiety of Formula (IV) forms an intermolecular covalent bond. In aspect, the moiety of Formula (V) forms an intermolecular covalent bond. In aspects, the moiety of Formula (VI) forms an intermolecular covalent bond. In aspect, the moieties of Formula (IV) and (V) form intermolecular covalent bonds. In aspect, the moieties of Formula (IV) and (VI) form intermolecular covalent bonds. In aspect, the moieties of Formula (V) and (VI) form intermolecular covalent bonds. In aspect, the moieties of Formula (IV), (V), and (VI) form intermolecular covalent bonds.

In aspects, the disclosure provides a protein of Formula (I), Formula (II), or Formula (III):

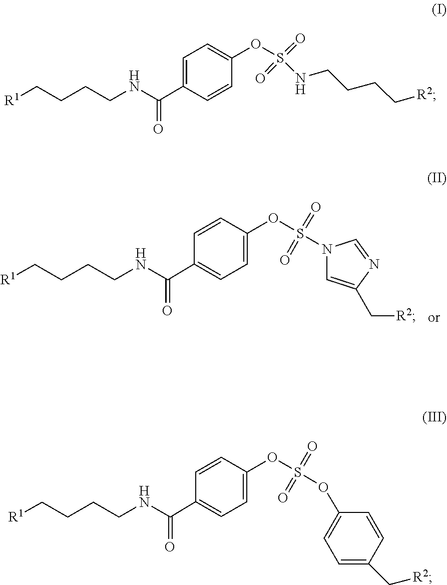

wherein R¹ and R² are each independently a peptidyl moiety that are joined together, i.e., the protein of Formula (I), (II), and (III) comprises an intramolecular covalent bond. In aspects, the protein is Formula (I). In aspects, the protein is Formula (II). In aspects, the protein is Formula (III). In aspects, the peptidyl moiety of R¹ and the peptidyl moiety of R² comprise a protein α-strand. In aspects, the peptidyl moiety of R¹ and the peptidyl moiety of R² comprise a protein β-strand. In aspects, the peptidyl moiety of R¹ comprises a protein α-strand and the peptidyl moiety of R² comprises a protein β-strand. In aspects, the peptidyl moiety of R¹ comprises a protein β-strand and the peptidyl moiety of R² comprises a protein α-strand.

In aspects, the disclosure provides a protein of Formula (I), Formula (II), or Formula (III):

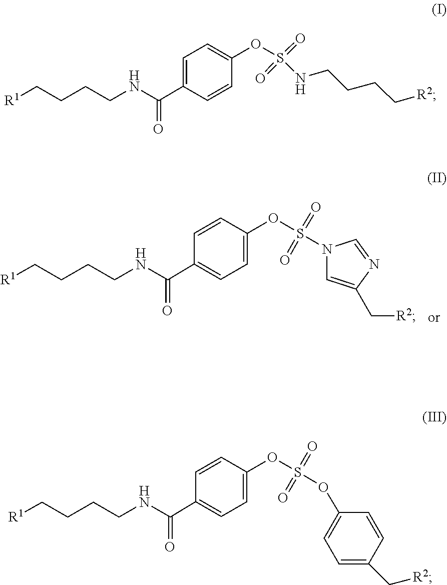

wherein R¹ is a peptidyl moiety of a first protein and R² is a peptidyl moiety of a second protein, i.e., there is an intermolecular covalent bond between two proteins. In aspects, the intermolecular bond is between two different proteins. In aspects, the intermolecular bond is between two of the same proteins (e.g., two proteins having the same amino acid sequence that are intermolecularly bonded). In aspects the first protein is covalently bonded to the second protein via the moiety of Formula (IV) to form an intermolecularly bonded protein of Formula (I). In aspects the first protein is covalently bonded to the second protein via the moiety of Formula (V) to form an intermolecularly bonded protein of Formula (II). In aspects the first protein is covalently bonded to the second protein via the moiety of Formula (VI) to form an intermolecularly bonded protein of Formula (III). In aspects the first protein is covalently bonded to the second protein via the moiety of Formula (IV) and the moiety of Formula (IV). In aspects the first protein is covalently bonded to the second protein via the moiety of Formula (IV) and the moiety of Formula (VI). In aspects the first protein is covalently bonded to the second protein via the moiety of Formula (V) and the moiety of Formula (VI). In aspects the first protein is covalently bonded to the second protein via the moiety of Formula (IV), the moiety of Formula (V), and the moiety of Formula (VI). In aspects, the first protein is a hormone and the second protein is the receptor for the hormone. In aspects, the first protein is an antibody or an antibody variant, and the second protein is a membrane receptor. In aspects, the first protein is an antibody and the second protein is a membrane receptor. In aspects, the first protein is an antibody variant and the second protein is a membrane receptor. In aspects, the first protein is an antibody, an antigen-binding fragment, a single-chain variable fragment, a single-domain antibody, or an affibody and the second protein is membrane receptor. In aspects, the first protein is an antibody-binding fragment and the second protein is membrane receptor. In aspects, the first protein is a single-chain variable fragment and the second protein is membrane receptor. In aspects, the first protein is a single-domain antibody and the second protein is membrane receptor. In aspects, the first protein is an affibody and the second protein is membrane receptor. In aspects, the first protein is a single-domain antibody and the second protein is hormone receptor. In aspects, the peptidyl moiety R¹ and R² comprise a protein α-strand. In aspects, the peptidyl moiety R¹ and R² comprise a protein β-strand. In aspects, the peptidyl moiety R¹ comprises a protein α-strand and the peptidyl moiety R² comprises a protein β-strand. In aspects, the peptidyl moiety R¹ comprises a protein β-strand and the peptidyl moiety R² comprises a protein α-strand. In aspects, R¹ is an antibody or an antibody variant, and R² is a membrane receptor. In aspects, R¹ is an antibody and R² is a membrane receptor. In aspects, R¹ is an antibody variant and R² is a membrane receptor. In aspects, R¹ is an antibody, an antigen-binding fragment, a single-chain variable fragment, a single-domain antibody, or an affibody, and R² is a membrane receptor. In aspects, R¹ is an antigen-binding fragment and R² is a membrane receptor. In aspects, R¹ is a single-chain variable fragment and R² is a membrane receptor. In aspects, R¹ is a single-domain antibody and R² is a membrane receptor. In aspects, R¹ is an affibody and R² is a membrane receptor. In aspects, R¹ is a membrane receptor and R² is an antibody or an antibody variant. In aspects, R¹ is a membrane receptor and R² is an antibody. In aspects, R¹ is a membrane receptor and R² is an antibody variant. In aspects, R¹ is a membrane receptor and R² is an antibody, an antigen-binding fragment, a single-chain variable fragment, a single-domain antibody, or an affibody. In aspects, R¹ is a membrane receptor and R² is an antigen-binding fragment. In aspects, R¹ is a membrane receptor and R² is a single-chain variable fragment. In aspects, R¹ is a membrane receptor and R² is a single-domain antibody. In aspects, R¹ is a membrane receptor and R² is an affibody.

In aspects, the protein conjugates may comprise three or more different and/or separate proteins. For example, the first protein is covalently bonded to the second protein via a moiety of Formula (IV), a moiety of Formula (V), a moiety of Formula (VI), or a combination of two or more thereof; and the second protein is covalently bonded to a third protein via a moiety of Formula (IV), a moiety of Formula (V), a moiety of Formula (VI), or a combination of two or more thereof. As another example, the first protein is covalently bonded to the second protein via a moiety of Formula (IV), a moiety of Formula (V), a moiety of Formula (VI), or a combination of two or more thereof; and the first protein is also covalently bonded to a third protein via a moiety of Formula (IV), a moiety of Formula (V), a moiety of Formula (VI), or a combination of two or more thereof. In each of these aspects, the first protein, the second protein, and the third protein may each optionally further comprise a moiety of Formula (IV), a moiety of Formula (V), a moiety of Formula (VI), or a combination of two or more thereof, wherein the peptidyl moiety of R¹ and R² form intramolecular bonds within the first protein, the second protein, or the third protein, respectively.

In embodiments, the disclosure provides a small molecule moiety, a membrane receptor, an antibody, an antigen-binding fragment, a single-chain variable fragment, a single-domain antibody, or an affibody comprising an unnatural amino acid; wherein the unnatural amino acid has a side chain of Formula (F):

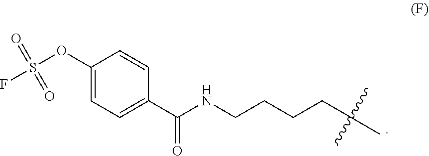 [SMILES: *CCCCNC(=O)c1ccc(OS(=O)(=O)F)cc1]

In embodiments, the disclosure provides an antibody, an antigen-binding fragment, a single-chain variable fragment, a single-domain antibody, or an affibody comprising the unnatural amino acid side chain of Formula (F). In embodiments, the disclosure provides a membrane receptor comprising the unnatural amino acid side chain of Formula (F). In embodiments, the disclosure provides a small molecule moiety comprising the unnatural amino acid side chain of Formula (F). In embodiments, the disclosure provides an antibody comprising the unnatural amino acid side chain of Formula (F). In embodiments, the disclosure provides an antigen-binding fragment, a single-chain variable fragment comprising the unnatural amino acid side chain of Formula (F). In embodiments, the disclosure provides a single-chain variable fragment comprising the unnatural amino acid side chain of Formula (F). In embodiments, the disclosure provides a single-domain antibody comprising the unnatural amino acid side chain of Formula (F). In embodiments, the disclosure provides an affibody comprising the unnatural amino acid side chain of Formula (F).

In embodiments, the biomolecules and proteins described herein comprises a membrane receptor. In embodiments, the membrane receptor is a programmed cell death protein 1 (PD-1) receptor, a programmed death ligand 1 (PD-L1) receptor, a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid SIP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor, or a combination of two or more thereof.

In embodiments, the membrane receptor is PD-1 receptor or PD-L1 receptor. In embodiments, the membrane receptor is PD-1 receptor. In embodiments, the membrane receptor is a PD-L1 receptor.

In embodiments, the membrane receptor is a receptor expressed on a cancer cell. In embodiments, the membrane receptor is a receptor overexpressed on a cancer cell relative to a control.

In embodiments, the membrane receptor is a G protein-coupled receptor. In embodiments, the membrane receptor is a receptor tyrosine kinase. In embodiments, the receptor protein is a an ErbB receptor. In embodiments, the membrane receptor is an epidermal growth factor receptor (EGFR). In embodiments, the membrane receptor is epidermal growth factor receptor 1 (HER1). In embodiments, the membrane receptor is epidermal growth factor receptor 2 (HER2). In embodiments, the membrane receptor is epidermal growth factor receptor 3 (HER3). In embodiments, the membrane receptor is epidermal growth factor receptor 4 (HER4).

In embodiments, the membrane receptor is EGFR. In embodiments, the membrane receptor is EGFR expressed on a cancer cell. In embodiments, the membrane receptor is EGFR that is overexpressed on a cancer cell relative to a control.

Provided herein is nanobody 7D12 modified with FSK or FSY. Nanobody 7D12 is set forth as SEQ ID NO:88, wherein CDR1 is as set forth in SEQ ID NO:95, CDR2 is as set forth in SEQ ID NO:96, and CDR3 is as set forth in SEQ ID NO:97.

SEQ ID NO: 88
QVKLEESGGG SVQTGGSLRL TCAASGRTSR SYGMGWFRQA
PGKEREFVSG ISWRGDSTGY ADSVKGRFTI SRDNAKNTVD
LQMNSLKPED TAIYYCAAAA GSAWYGTLYE YDYWGQGTQV
TVSS
SEQ ID NO: 95 =
RTSRSYGMG
SEQ ID NO: 96 =
GISWRGDS
SEQ ID NO: 97 =
AAGSAWYGTLYEYDY

Provided herein is nanobody 7D12 wherein at least one amino acid in the nanobody is FSK. In embodiments, the nanobody comprises CDR1 as set forth in SEQ ID NO:95, CDR2 as set forth in SEQ ID NO:96, and CDR3 as set forth in SEQ ID NO:97; wherein the amino acid at the position corresponding to position 30 or position 31 is FSK In embodiments, the nanobody comprises CDR1 as set forth in SEQ ID NO:95, CDR2 as set forth in SEQ ID NO:96, and CDR3 as set forth in SEQ ID NO:97; wherein the amino acid at the position corresponding to position 30 is FSK (i.e., wherein position 30 corresponds to position 4 in SEQ ID NO:95). In embodiments, the nanobody comprises CDR1 as set forth in SEQ ID NO:95, CDR2 as set forth in SEQ ID NO:96, and CDR3 as set forth in SEQ ID NO:97; wherein the amino acid at the position corresponding to position 31 is FSK (i.e., wherein position 31 corresponds to position 5 in SEQ ID NO:95). In embodiments, the nanobody comprises CDR1 as set forth in SEQ ID NO:98, CDR2 as set forth in SEQ ID NO:96, and CDR3 as set forth in SEQ ID NO:97. In embodiments, the nanobody comprises CDR1 as set forth in SEQ ID NO:99, CDR2 as set forth in SEQ ID NO:96, and CDR3 as set forth in SEQ ID NO:97. In the sequences, X^{FSK} is FSK.

SEQ ID NO: 98 =
RTSX^{FSK}SYGMG
SEQ ID NO: 99 =
RTSRX^{FSK}YGMG

In embodiments, nanobody 7D12 has the amino acid sequence set forth in SEQ ID NO:35 or SEQ ID NO:88, wherein at least one amino acid in the amino acid sequence is FSK. In embodiments, nanobody 7D12 has the amino acid sequence set forth in SEQ ID NO:35, wherein at least one amino acid in the amino acid sequence is FSK. In embodiments, nanobody 7D12 has the amino acid sequence set forth in SEQ ID NO:88, wherein at least one amino acid in the amino acid sequence is FSK. In embodiments, nanobody 7D12 has the amino acid sequence set forth in SEQ ID NO:88, wherein the amino acid at the position corresponding to position 30 or position 31 is FSK. In embodiments, nanobody 7D12 has the amino acid sequence set forth in SEQ ID NO:88, wherein the amino acid at the position corresponding to position 30 is FSK (i.e., SEQ ID NO:89). In embodiments, nanobody 7D12 has the amino acid sequence set forth in SEQ ID NO:88, wherein the amino acid at the position corresponding to position 31 is FSK (i.e., SEQ ID NO:90).

In embodiments, the nanobody comprises SEQ ID NO:89. In embodiments, the nanobody is as set forth at SEQ ID NO:89. In embodiments, the nanobody has at least 85% sequence identity to SEQ ID NO:89. In embodiments, the nanobody has at least 90% sequence identity to SEQ ID NO:89. In embodiments, the nanobody has at least 92% sequence identity to SEQ ID NO:89. In embodiments, the nanobody has at least 94% sequence identity to SEQ ID NO:89. In embodiments, the nanobody has at least 95% sequence identity to SEQ ID NO:89. In embodiments, the nanobody has at least 96% sequence identity to SEQ ID NO:89. In embodiments, the nanobody has at least 98% sequence identity to SEQ ID NO:89. In embodiments where there is less than 100% sequence identity, the nanobody must contain FSK at a position corresponding to position 30 in SEQ ID NO:89. In embodiments when the nanobody has less than 100% sequence identity to SEQ ID NO:89, then the nanobody has 100% sequence identity to CDR1, CDR2, and CDR3 within SEQ ID NO:89. In SEQ ID NO:89, X^{FSK} is FSK.

SEQ ID NO: 89
QVKLEESGGG SVQTGGSLRL TCAASGRTSX^{FSK} SYGMGWFRQA
PGKEREFVSG ISWRGDSTGY ADSVKGRFTI SRDNAKNTVD
LQMNSLKPED TAIYYCAAAA GSAWYGTLYE YDYWGQGTQV
TVSS

In embodiments, the nanobody comprises SEQ ID NO:90. In embodiments, the nanobody is as set forth at SEQ ID NO:90. In embodiments, the nanobody has at least 85% sequence identity to SEQ ID NO:90. In embodiments, the nanobody has at least 90% sequence identity to SEQ ID NO:90. In embodiments, the nanobody has at least 92% sequence identity to SEQ ID NO:90. In embodiments, the nanobody has at least 94% sequence identity to SEQ ID NO:90. In embodiments, the nanobody has at least 95% sequence identity to SEQ ID NO:90. In embodiments, the nanobody has at least 96% sequence identity to SEQ ID NO:90. In embodiments, the nanobody has at least 98% sequence identity to SEQ ID NO:90. In embodiments where there is less than 100% sequence identity, the nanobody must contain FSK at a position corresponding to position 31 in SEQ ID NO:90. In embodiments when the nanobody has less than 100% sequence identity to SEQ ID NO:90, then the nanobody has 100% sequence identity to CDR1, CDR2, and CDR3 within SEQ ID NO:90. In SEQ ID NO:90, X^{FSK} is FSK.

SEQ ID NO: 90
QVKLEESGGG SVQTGGSLRL TCAASGRTSR X^{FSK}YGMGWFRQA
PGKEREFVSG ISWRGDSTGY ADSVKGRFTI SRDNAKNTVD
LQMNSLKPED TAIYYCAAAA GSAWYGTLYE YDYWGQGTQV
TVSS

Provided herein is nanobody 7D12 wherein at least one amino acid in the nanobody is FSY. In embodiments, nanobody 7D12 comprises CDR1 as set forth in SEQ ID NO:95, CDR2 as set forth in SEQ ID NO:96, and CDR3 as set forth in SEQ ID NO:97; wherein the amino acid at the position corresponding to position 109 or position 113 is FSY. In embodiments, nanobody 7D12 comprises CDR1 as set forth in SEQ ID NO:95, CDR2 as set forth in SEQ ID NO:96, and CDR3 as set forth in SEQ ID NO:97; wherein the amino acid at the position corresponding to position 109 is FSY (i.e., wherein position 109 corresponds to position 11 in SEQ ID NO:97). In embodiments, nanobody 7D12 comprises CDR1 as set forth in SEQ ID NO:95, CDR2 as set forth in SEQ ID NO:96, and CDR3 as set forth in SEQ ID NO:100. In embodiments, nanobody 7D12 comprises CDR1 as set forth in SEQ ID NO:95, CDR2 as set forth in SEQ ID NO:96, and CDR3 as set forth in SEQ ID NO:101. In the sequences, X^{FSY} is FSY.

SEQ ID NO: 100 =
AAGSAWYGTLX^{FSY}EYDY
SEQ ID NO: 101 =
AAGSAWYGTLYEYDX^{FSY}

In embodiments, nanobody 7D12 has the amino acid sequence set forth in SEQ ID NO:35 or SEQ ID NO:88, wherein at least one amino acid in the amino acid sequence is FSY. In embodiments, nanobody 7D12 has the amino acid sequence set forth in SEQ ID NO:35, wherein at least one amino acid in the amino acid sequence is FSY. In embodiments, nanobody 7D12 has the amino acid sequence set forth in SEQ ID NO:88, wherein at least one amino acid in the amino acid sequence is FSY. In embodiments, nanobody 7D12 has the amino acid sequence set forth in SEQ ID NO:88, wherein the amino acid at the position corresponding to position 1, position 109, position 113, or position 116 is FSY. In embodiments, nanobody 7D12 has the amino acid sequence set forth in SEQ ID NO:88, wherein the amino acid at the position corresponding to position 1 is FSY (i.e., SEQ ID NO:91). In embodiments, nanobody 7D12 has the amino acid sequence set forth in SEQ ID NO:88, wherein the amino acid at the position corresponding to position 109 is FSY (i.e., SEQ ID NO:92). In embodiments, nanobody 7D12 has the amino acid sequence set forth in SEQ ID NO:88, wherein the amino acid at the position corresponding to position 113 is FSY (i.e., SEQ ID NO:93). In embodiments, nanobody 7D12 has the amino acid sequence set forth in SEQ ID NO:88, wherein the amino acid at the position corresponding to position 116 is FSY (i.e., SEQ ID NO:94).

In embodiments, the nanobody comprises SEQ ID NO:91. In embodiments, the nanobody is as set forth at SEQ ID NO:91. In embodiments, the nanobody has at least 85% sequence identity to SEQ ID NO:91. In embodiments, the nanobody has at least 90% sequence identity to SEQ ID NO:91. In embodiments, the nanobody has at least 92% sequence identity to SEQ ID NO:91. In embodiments, the nanobody has at least 94% sequence identity to SEQ ID NO:91. In embodiments, the nanobody has at least 95% sequence identity to SEQ ID NO:91. In embodiments, the nanobody has at least 96% sequence identity to SEQ ID NO:91. In embodiments, the nanobody has at least 98% sequence identity to SEQ ID NO:91. In embodiments where there is less than 100% sequence identity, the nanobody must contain FSY at a position corresponding to position 1 in SEQ ID NO:91. In embodiments when the nanobody has less than 100% sequence identity to SEQ ID NO:91, then the nanobody has 100% sequence identity to CDR1, CDR2, and CDR3 within SEQ ID NO:91, and the nanobody has FSY at a position corresponding to position 1 in SEQ ID NO:91. In SEQ ID NO:91, X^{FSY} is FSY.

SEQ ID NO: 91
X^{FSY}VKLEESGGG SVQTGGSLRL TCAASGRTSR SYGMGWFRQA
PGKEREFVSG ISWRGDSTGY ADSVKGRFTI SRDNAKNTVD
LQMNSLKPED TAIYYCAAAA GSAWYGTLYE YDYWGQGTQV
TVSS

In embodiments, the nanobody comprises SEQ ID NO:92. In embodiments, the nanobody is as set forth at SEQ ID NO:92. In embodiments, the nanobody having at least 85% sequence identity to SEQ ID NO:92. In embodiments, the nanobody has at least 90% sequence identity to SEQ ID NO:92. In embodiments, the nanobody has at least 92% sequence identity to SEQ ID NO:92. In embodiments, the nanobody has at least 94% sequence identity to SEQ ID NO:92. In embodiments, the nanobody has at least 95% sequence identity to SEQ ID NO:92. In embodiments, the nanobody has at least 96% sequence identity to SEQ ID NO:92. In embodiments, the nanobody has at least 98% sequence identity to SEQ ID NO:92. In embodiments where there is less than 100% sequence identity, the nanobody must contain FSY at a position corresponding to position 109 in SEQ ID NO:92. In embodiments when the nanobody has less than 100% sequence identity to SEQ ID NO:92, then the nanobody has 100% sequence identity to CDR1, CDR2, and CDR3 within SEQ ID NO:92. In SEQ ID NO:92, X^{FSY} is FSY.

SEQ ID NO: 92
QVKLEESGGG SVQTGGSLRL TCAASGRTSR SYGMGWFRQA
PGKEREFVSG ISWRGDSTGY ADSVKGRFTI SRDNAKNTVD
LQMNSLKPED TAIYYCAAAA GSAWYGTLX^{FSY}E YDYWGQGTQV
TVSS

In embodiments, the nanobody comprises SEQ ID NO:93. In embodiments, the nanobody is as set forth at SEQ ID NO:93. In embodiments, the nanobody has at least 85% sequence identity to SEQ ID NO:93. In embodiments, the nanobody has at least 90% sequence identity to SEQ ID NO:93. In embodiments, the nanobody has at least 92% sequence identity to SEQ ID NO:93. In embodiments, the nanobody has at least 94% sequence identity to SEQ ID NO:93. In embodiments, the nanobody has at least 95% sequence identity to SEQ ID NO:93. In embodiments, the nanobody has at least 96% sequence identity to SEQ ID NO:93. In embodiments, the nanobody has at least 98% sequence identity to SEQ ID NO:93. In embodiments where there is less than 100% sequence identity, the nanobody must contain FSY at a position corresponding to position 113 in SEQ ID NO:93. In embodiments when the nanobody has less than 100% sequence identity to SEQ ID NO:93, then the nanobody has 100% sequence identity to CDR1, CDR2, and CDR3 within SEQ ID NO:93. In SEQ ID NO:93, X^{F}SY is FSY.

SEQ ID NO: 93
QVKLEESGGG SVQTGGSLRL TCAASGRTSR SYGMGWFRQA
PGKEREFVSG ISWRGDSTGY ADSVKGRFTI SRDNAKNTVD
LQMNSLKPED TAIYYCAAAA GSAWYGTLYE YDX^{FSY}WGQGTQV
TVSS

In embodiments, the nanobody comprises SEQ ID NO:94. In embodiments, the nanobody is as set forth at SEQ ID NO:94. In embodiments, the nanobody has at least 85% sequence identity to SEQ ID NO:94. In embodiments, the nanobody has at least 90% sequence identity to SEQ ID NO:94. In embodiments, the nanobody has at least 92% sequence identity to SEQ ID NO:94. In embodiments, the nanobody has at least 94% sequence identity to SEQ ID NO:94. In embodiments, the nanobody has at least 95% sequence identity to SEQ ID NO:94. In embodiments, the nanobody has at least 96% sequence identity to SEQ ID NO:94. In embodiments, the nanobody has at least 98% sequence identity to SEQ ID NO:94. In embodiments where there is less than 100% sequence identity, the nanobody must contain FSY at a position corresponding to position 116 in SEQ ID NO:94. In embodiments when the nanobody has less than 100% sequence identity to SEQ ID NO:94, then the nanobody has 100% sequence identity to CDR1, CDR2, and CDR3 within SEQ ID NO:94, and the nanobody has FSY at a position corresponding to position 116 in SEQ ID NO:94. In SEQ ID NO:94, X^{FSY} is FSY.

SEQ ID NO: 94
QVKLEESGGG SVQTGGSLRL TCAASGRTSR SYGMGWFRQA
PGKEREFVSG ISWRGDSTGY ADSVKGRFTI SRDNAKNTVD
LQMNSLKPED TAIYYCAAAA GSAWYGTLYE YDYWGX^{FSY}GTQV
TVSS

In embodiments, the disclosure provides a pharmaceutical composition comprising nanobody 7D12 wherein at least one amino acid in the amino acid sequence is FSK (including embodiments as described herein) and a pharmaceutically acceptable excipient. In embodiments, the pharmaceutical composition comprises SEQ ID NO:89 (including embodiments thereof) and a pharmaceutically acceptable carrier. In embodiments, the pharmaceutical composition comprises SEQ ID NO:90 (including embodiments thereof) and a pharmaceutically acceptable excipient. In embodiments, the pharmaceutical composition comprises a nanobody comprising CDR1 as set forth in SEQ ID NO:98, CDR2 as set forth in SEQ ID NO:96, and CDR3 as set forth in SEQ ID NO:97, and a pharmaceutically acceptable excipient. In embodiments, the pharmaceutical composition comprises a nanobody comprising CDR1 as set forth in SEQ ID NO:99, CDR2 as set forth in SEQ ID NO:96, and CDR3 as set forth in SEQ ID NO:97, and a pharmaceutically acceptable excipient.

In embodiments, the disclosure provides a biomolecule conjugate comprising nanobody 7D12 wherein at least one amino acid in the amino acid sequence is FSK (including embodiments as described herein) covalently bonded via the amino acid side chain of FSK to a lysine, histidine, or tyrosine amino acid in the EGFR protein. In embodiments, the disclosure provides a biomolecule conjugate comprising nanobody 7D12 wherein at least one amino acid in the amino acid sequence is FSK (including embodiments as described herein) covalently bonded via the amino acid side chain of FSK to a lysine in the EGFR protein. In embodiments, the disclosure provides a biomolecule conjugate comprising nanobody 7D12 wherein at least one amino acid in the amino acid sequence is FSK (including embodiments as described herein) covalently bonded via the amino acid side chain of FSK to a histidine in the EGFR protein. In embodiments, the disclosure provides a biomolecule conjugate comprising nanobody 7D12 wherein at least one amino acid in the amino acid sequence is FSK (including embodiments as described herein) covalently bonded via the amino acid side chain of FSK to a tyrosine in the EGFR protein. In embodiments, the biomolecule conjugate comprises SEQ ID NO:89 (including embodiments thereof) covalently bonded via FSK to a lysine, histidine, or tyrosine amino acid in EGFR. In embodiments, the biomolecule conjugate comprises SEQ ID NO:89 (including embodiments thereof) covalently bonded via FSK to a lysine amino acid in EGFR. In embodiments, the biomolecule conjugate comprises SEQ ID NO:89 (including embodiments thereof) covalently bonded via FSK to a histidine amino acid in EGFR. In embodiments, the biomolecule conjugate comprises SEQ ID NO:89 (including embodiments thereof) covalently bonded via FSK to a tyrosine amino acid in EGFR. In embodiments, the biomolecule conjugate comprises SEQ ID NO:90 (including embodiments thereof) covalently bonded via FSK to a lysine, histidine, or tyrosine amino acid in EGFR. In embodiments, the biomolecule conjugate comprises SEQ ID NO:90 (including embodiments thereof) covalently bonded via FSK to a lysine amino acid in EGFR. In embodiments, the biomolecule conjugate comprises SEQ ID NO:90 (including embodiments thereof) covalently bonded via FSK to a histidine amino acid in EGFR. In embodiments, the biomolecule conjugate comprises SEQ ID NO:90 (including embodiments thereof) covalently bonded via FSK to a tyrosine amino acid in EGFR.

In embodiments, the disclosure provides a pharmaceutical composition comprising nanobody 7D12 wherein at least one amino acid in the amino acid sequence is FSY (including embodiments as described herein) and a pharmaceutically acceptable carrier. In embodiments, the pharmaceutical composition comprises SEQ ID NO:91 (including embodiments thereof) and a pharmaceutically acceptable carrier. In embodiments, the pharmaceutical composition comprises SEQ ID NO:92 (including embodiments thereof) and a pharmaceutically acceptable carrier. In embodiments, the pharmaceutical composition comprises SEQ ID NO:93 (including embodiments thereof) and a pharmaceutically acceptable carrier. In embodiments, the pharmaceutical composition comprises SEQ ID NO:94 (including embodiments thereof) and a pharmaceutically acceptable carrier. In embodiments, the pharmaceutical composition comprises a nanobody comprising CDR1 as set forth in SEQ ID NO:95, CDR2 as set forth in SEQ ID NO:96, and CDR3 as set forth in SEQ ID NO:100, and a pharmaceutically acceptable excipient. In embodiments, the pharmaceutical composition comprises a nanobody comprising CDR1 as set forth in SEQ ID NO:95, CDR2 as set forth in SEQ ID NO:96, and CDR3 as set forth in SEQ ID NO:101, and a pharmaceutically acceptable excipient.

In embodiments, the disclosure provides a biomolecule conjugate comprising nanobody 7D12 wherein at least one amino acid in the amino acid sequence is FSY (including embodiments as described herein) covalently bonded via the amino acid side chain of FSY to a lysine, histidine, or tyrosine amino acid in the EGFR protein. In embodiments, the disclosure provides a biomolecule conjugate comprising nanobody 7D12 wherein at least one amino acid in the amino acid sequence is FSY (including embodiments as described herein) covalently bonded via the amino acid side chain of FSY to a lysine in the EGFR protein. In embodiments, the disclosure provides a biomolecule conjugate comprising nanobody 7D12 wherein at least one amino acid in the amino acid sequence is FSY (including embodiments as described herein) covalently bonded via the amino acid side chain of FSY to a histidine in the EGFR protein. In embodiments, the disclosure provides a biomolecule conjugate comprising nanobody 7D12 wherein at least one amino acid in the amino acid sequence is FSY (including embodiments as described herein) covalently bonded via the amino acid side chain of FSY to a tyrosine in the EGFR protein. In embodiments, the biomolecule conjugate comprises SEQ ID NO:91 (including embodiments thereof) covalently bonded via FSY to a lysine, histidine, or tyrosine amino acid in EGFR. In embodiments, the biomolecule conjugate comprises SEQ ID NO:91 (including embodiments thereof) covalently bonded via FSY to a lysine amino acid in EGFR. In embodiments, the biomolecule conjugate comprises SEQ ID NO:91 (including embodiments thereof) covalently bonded via FSY to a histidine amino acid in EGFR. In embodiments, the biomolecule conjugate comprises SEQ ID NO:91 (including embodiments thereof) covalently bonded via FSY to a tyrosine amino acid in EGFR. In embodiments, the biomolecule conjugate comprises SEQ ID NO:92 (including embodiments thereof) covalently bonded via FSY to a lysine, histidine, or tyrosine amino acid in EGFR. In embodiments, the biomolecule conjugate comprises SEQ ID NO:92 (including embodiments thereof) covalently bonded via FSY to a lysine amino acid in EGFR. In embodiments, the biomolecule conjugate comprises SEQ ID NO:92 (including embodiments thereof) covalently bonded via FSY to a histidine amino acid in EGFR. In embodiments, the biomolecule conjugate comprises SEQ ID NO:92 (including embodiments thereof) covalently bonded via FSY to a tyrosine amino acid in EGFR. In embodiments, the biomolecule conjugate comprises SEQ ID NO:93 (including embodiments thereof) covalently bonded via FSY to a lysine, histidine, or tyrosine amino acid in EGFR. In embodiments, the biomolecule conjugate comprises SEQ ID NO:93 (including embodiments thereof) covalently bonded via FSY to a lysine amino acid in EGFR. In embodiments, the biomolecule conjugate comprises SEQ ID NO:93 (including embodiments thereof) covalently bonded via FSY to a histidine amino acid in EGFR. In embodiments, the biomolecule conjugate comprises SEQ ID NO:93 (including embodiments thereof) covalently bonded via FSY to a tyrosine amino acid in EGFR. In embodiments, the biomolecule conjugate comprises SEQ ID NO:94 (including embodiments thereof) covalently bonded via FSY to a lysine, histidine, or tyrosine amino acid in EGFR. In embodiments, the biomolecule conjugate comprises SEQ ID NO:94 (including embodiments thereof) covalently bonded via FSY to a lysine amino acid in EGFR. In embodiments, the biomolecule conjugate comprises SEQ ID NO:94 (including embodiments thereof) covalently bonded via FSY to a histidine amino acid in EGFR. In embodiments, the biomolecule conjugate comprises SEQ ID NO:94 (including embodiments thereof) covalently bonded via FSY to a tyrosine amino acid in EGFR.

### Pyrrolysyl-tRNA Synthetase

As described herein, an unnatural amino acid (e.g., FSK) may be inserted into or replace a naturally occurring amino acid in a biomolecule (e.g., protein). In order for the unnatural amino acid to be inserted or replace an amino acid in a biomolecule (e.g., protein), it must be capable of being incorporated during proteinogenesis. Thus, the unnatural amino acid must be present on a transfer RNA molecule (tRNA) such that it may be used in translation. Loading of amino acids occurs via an aminoacyl-tRNA synthetase, which is an enzyme that facilitates the attachment of appropriate amino acids to tRNA molecules. However, the attachment of unnatural amino acids to tRNA may not necessarily be accomplished by the naturally occurring aminoacyl-tRNA synthetase. Engineered aminoacyl-tRNA synthetases (e.g., mutant pyrrolysyl-tRNA synthetase (PylRS)) may be useful for attaching unnatural amino acids to tRNA. A PylRS mutant library was generated, Compared to previously described PylRS mutant library, the PylRS mutant library generated herein was constructed using the new small-intelligent mutagenesis approach that allows a greater number of amino acid residues to be mutated simultaneously (e.g., 10 amino acid residues). Out of the clones selected and screened in total, four PylRS mutant were identified that were capable of attaching FSK, and one PylRS was particularly effective in attaching FSK.

The disclosure provides a mutant pyrrolysyl-tRNA synthetase, including at least 5 amino acid residues substitutions within the substrate-binding site of the mutant pyrrolysyl-tRNA synthetase. In aspects, the mutant pyrrolysyl-tRNA synthetase comprises at least 5 amino acid residues substitutions in the amino acid sequence of SEQ ID NO:1. In aspects, the at least 5 amino acid substitutions occur at the residues tyrosine at position 126, methionine at position 129, valine at position 168, histidine at position 227, and tyrosine at position 228 as set forth in the amino acid sequence of SEQ ID NO:1. In aspects, the at least 5 amino acid substitutions are: (i) Y126G; (ii) M129A; (iii) V168F; (iv) H227T, H227S, or H227I; and (v) Y228P, in the amino acid sequence of SEQ ID NO:1. In aspects, the mutant pyrrolysyl-tRNA synthetase comprises at least 6 amino acid residues substitutions in the amino acid sequence of SEQ ID NO:1. In aspects, the at least 6 amino acid substitutions occur at the residues tyrosine at position 126, methionine at position 129, valine at position 168, histidine at position 227, tyrosine at position 228, and lysine at position 229 as set forth in the amino acid sequence of SEQ ID NO:1. In aspects, the at least 6 amino acid residues substitutions are: (i) Y126G; (ii) M129A; (iii) V168F; (iv) H227T, H227S, or H227I; (v) Y228P; and (vi) L229V or L229I, in the amino acid sequence of SEQ ID NO:1.

In embodiments, the mutant pyrrolysyl-tRNA synthetase is encoded by the nucleic acid sequence of SEQ ID NO:2. In aspects, the mutant pyrrolysyl-tRNA synthetase is encoded by a nucleic acid sequence including the sequence of SEQ ID NO:2. In aspects, the mutant pyrrolysyl-tRNA synthetase is encoded by a nucleic acid sequence that is at least 80%, 85%, 90%, 91%, 92%, 93%, 94%, 95%, 96%, 97%, 98%, 99%, or 100% identity to SEQ ID NO:2. In aspects, the mutant pyrrolysyl-tRNA synthetase has an amino acid sequence that has at least 80% identity to SEQ ID NO:2. In aspects, the mutant pyrrolysyl-tRNA synthetase has an amino acid sequence that has at least 85% identity to SEQ ID NO:2. In aspects, the mutant pyrrolysyl-tRNA synthetase has an amino acid sequence that has at least 90% identity to SEQ ID NO:2. In aspects, the mutant pyrrolysyl-tRNA synthetase has an amino acid sequence that has at least 95% identity to SEQ ID NO:2. In aspects, the mutant pyrrolysyl-tRNA synthetase has an amino acid sequence that has at least 98% identity to SEQ ID NO:2. SEQ ID NO:2 is alternatively referred to as FSKRS.

In embodiments, the mutant pyrrolysyl-tRNA synthetase is encoded by the nucleic acid sequence of SEQ ID NO:86. In aspects, the mutant pyrrolysyl-tRNA synthetase is encoded by a nucleic acid sequence including the sequence of SEQ ID NO:86. In aspects, the mutant pyrrolysyl-tRNA synthetase is encoded by a nucleic acid sequence that is at least 80%, 85%, 90%, 91%, 92%, 93%, 94%, 95%, 96%, 97%, 98%, 99%, or 100% identity to SEQ ID NO:86. In aspects, the mutant pyrrolysyl-tRNA synthetase has an amino acid sequence that has at least 80% identity to SEQ ID NO:86. In aspects, the mutant pyrrolysyl-tRNA synthetase has an amino acid sequence that has at least 85% identity to SEQ ID NO:86. In aspects, the mutant pyrrolysyl-tRNA synthetase has an amino acid sequence that has at least 90% identity to SEQ ID NO:86. In aspects, the mutant pyrrolysyl-tRNA synthetase has an amino acid sequence that has at least 95% identity to SEQ ID NO:86. In aspects, the mutant pyrrolysyl-tRNA synthetase has an amino acid sequence that has at least 98% identity to SEQ ID NO:86. In aspects, when the pyrrolysyl-tRNA synthetase has less than 100% sequence identity to SEQ ID NO:86, the first seven amino acids at the N-terminus are always MH₆. SEQ ID NO:86 is alternatively referred to as FSKRSNThis.

In embodiments, the mutant pyrrolysyl-tRNA synthetase is encoded by the nucleic acid sequence of SEQ ID NO:87. In aspects, the mutant pyrrolysyl-tRNA synthetase is encoded by a nucleic acid sequence including the sequence of SEQ ID NO:87. In aspects, the mutant pyrrolysyl-tRNA synthetase is encoded by a nucleic acid sequence that is at least 80%, 85%, 90%, 91%, 92%, 93%, 94%, 95%, 96%, 97%, 98%, 99%, or 100% identity to SEQ ID NO:87. In aspects, the mutant pyrrolysyl-tRNA synthetase has an amino acid sequence that has at least 80% identity to SEQ ID NO:87. In aspects, the mutant pyrrolysyl-tRNA synthetase has an amino acid sequence that has at least 85% identity to SEQ ID NO:87. In aspects, the mutant pyrrolysyl-tRNA synthetase has an amino acid sequence that has at least 90% identity to SEQ ID NO:87. In aspects, the mutant pyrrolysyl-tRNA synthetase has an amino acid sequence that has at least 95% identity to SEQ ID NO:87. In aspects, the mutant pyrrolysyl-tRNA synthetase has an amino acid sequence that has at least 98% identity to SEQ ID NO:87. In aspects, when the pyrrolysyl-tRNA synthetase has less than 100% sequence identity to SEQ ID NO:87, the last six amino acids at the C-terminus are always histidine. SEQ ID NO:87 is alternatively referred to as FSKRSCThis.

### Vectors

It is contemplated that the compositions (e.g., mutant pyrrolysyl-tRNA synthetase, tRNA^{Pyl}) provided herein may be delivered to cells using methods well known in the art. Thus, in an aspect is provided a vector including a nucleic acid sequence encoding a mutant pyrrolysyl-tRNA synthetase as described herein, including embodiments thereof. In aspects, the vector comprises a nucleic acid sequence encoding a mutant pyrrolysyl-tRNA synthetase that comprises at least 5 amino acid residues substitutions within the substrate-binding site of the mutant pyrrolysyl-tRNA synthetase. In aspects, the vector further includes a nucleic acid sequence encoding tRNA^{Pyl}. In aspects, the vector comprises a nucleic acid sequence encoding a mutant pyrrolysyl-tRNA synthetase, including at least 5 amino acid residues substitutions within the substrate-binding site of the mutant pyrrolysyl-tRNA synthetase. In aspects, the vector comprises a nucleic acid sequence encoding a mutant pyrrolysyl-tRNA synthetase comprising at least 5 amino acid residues substitutions in the amino acid sequence of SEQ ID NO:1. In aspects, the at least 5 amino acid substitutions occur at the residues tyrosine at position 126, methionine at position 129, valine at position 168, histidine at position 227, and tyrosine at position 228 as set forth in the amino acid sequence of SEQ ID NO:1. In aspects, the at least 5 amino acid substitutions are: (i) Y126G; (ii) M129A; (iii) V168F; (iv) H227T, H227S, or H227I; and (v) Y228P, in the amino acid sequence of SEQ ID NO:1. In aspects, the mutant pyrrolysyl-tRNA synthetase comprises at least 6 amino acid residues substitutions in the amino acid sequence of SEQ ID NO:1. In aspects, the at least 5 amino acid substitutions occur at the residues tyrosine at position 126, methionine at position 129, valine at position 168, histidine at position 227, tyrosine at position 228, and lysine at position 229 as set forth in the amino acid sequence of SEQ ID NO:1. In aspects, the at least 6 amino acid residues substitutions are: (i) Y126G; (ii) M129A; (iii) V168F; (iv) H227T, H227S, or H227I; (v) Y228P; and (vi) L229V or L229I, in the amino acid sequence of SEQ ID NO:1. In aspects, the vector comprises a nucleic acid sequence encoding the amino acid sequence of SEQ ID NO:2. In aspects, the vector comprises a nucleic acid sequence encoding the amino acid sequence of SEQ ID NO:86. In aspects, the vector comprises a nucleic acid sequence encoding the amino acid sequence of SEQ ID NO:87. In aspects, the vector further includes a nucleic acid sequence encoding tRNA^{Pyl}.

In embodiments, the nucleic acid sequence encoding tRNA^{Pyl} is: GGGGGACGGTCCGGCGACCAGCGGGTCTCTAAAACCTAGCCAGCGGGGTTCGACGC CCCGGTCTCTCGCCA (SEQ ID NO:3). In aspects, the nucleic acid sequence encoding tRNA^{Pyl} comprises the sequence set forth in SEQ ID NO:3. In aspects, the nucleic acid sequence encoding tRNA^{Pyl} has a sequence that has at least 80%, 85%, 90%, 91%, 92%, 93%, 94%, 95%, 96%, 97%, 98%, 99%, or 100% sequence identity to SEQ ID NO:3. In aspects, the nucleic acid sequence encoding tRNA^{Pyl} has a sequence that has at least 80% sequence identity to SEQ ID NO:3. In aspects, the nucleic acid sequence encoding tRNA^{Pyl} has a sequence that has at least 85% sequence identity to SEQ ID NO:3. In aspects, the nucleic acid sequence encoding tRNA^{Pyl} has a sequence that has at least 90% sequence identity to SEQ ID NO:3. In aspects, the nucleic acid sequence encoding tRNA^{Pyl} has a sequence that has at least 95% sequence identity to SEQ ID NO:3. In aspects, the nucleic acid sequence encoding tRNA^{Pyl} has a sequence that has at least 98% sequence identity to SEQ ID NO:3.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. In embodiments, the disclosure provides a genome of a cell comprising a nucleic acid sequence encoding the pyrrolysyl-tRNA synthetase described herein (e.g., SEQ ID NO:1, SEQ ID NO:2, SEQ ID NO:86, or SEQ ID NO:87, including embodiments and aspects thereof). In embodiments, the disclosure provides a genome of a cell comprising a nucleic acid sequence encoding the pyrrolysyl-tRNA synthetase described herein (e.g., SEQ ID NO:1, SEQ ID NO:2, SEQ ID NO:86, or SEQ ID NO:87, including embodiments and aspects thereof) and a nucleic acid encoding tRNA^{Pyl} (e.g., SEQ ID NO:3, including embodiments and aspects thereof). Moreover, certain vectors are capable of directing the expression of genes to which they are operatively linked. Such vectors are referred to herein as “expression vectors.” In general, expression vectors of utility in recombinant DNA techniques are often in the form of plasmids. The terms “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Exemplary vectors that can be used include, but are not limited to, pEvol vector, pMP vector, pET vector, pTak vector, pBad vector.

### Complexes

In an aspect is provided a complex including a mutant pyrrolysyl-tRNA synthetase as described herein, including embodiments thereof; and fluorosulfonyloxybenzoyl-L-lysine (FSK) of Formula (A):

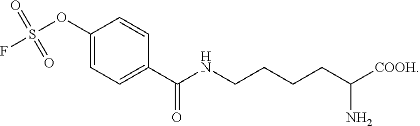 [SMILES: NC(CCCCNC(=O)c1ccc(OS(=O)(=O)F)cc1)C(=O)O]

In aspects, the complex comprises a mutant pyrrolysyl-tRNA synthetase, including at least 5 amino acid residues substitutions within the substrate-binding site of the mutant pyrrolysyl-tRNA synthetase. In aspects, the complex comprises a nucleic acid sequence encoding a mutant pyrrolysyl-tRNA synthetase comprising at least 5 amino acid residues substitutions in the amino acid sequence of SEQ ID NO:1. In aspects, the at least 5 amino acid substitutions occur at the residues tyrosine at position 126, methionine at position 129, valine at position 168, histidine at position 227, and tyrosine at position 228 as set forth in the amino acid sequence of SEQ ID NO:1. In aspects, the at least 5 amino acid substitutions are: (i) Y126G; (ii) M129A; (iii) V168F; (iv) H227T, H227S, or H227I; and (v) Y228P, in the amino acid sequence of SEQ ID NO:1. In aspects, the mutant pyrrolysyl-tRNA synthetase comprises at least 6 amino acid residues substitutions in the amino acid sequence of SEQ ID NO:1. In aspects, the at least 6 amino acid substitutions occur at the residues tyrosine at position 126, methionine at position 129, valine at position 168, histidine at position 227, tyrosine at position 228, and lysine at position 229 as set forth in the amino acid sequence of SEQ ID NO:1. In aspects, the at least 6 amino acid residues substitutions are: (i) Y126G; (ii) M129A; (iii) V168F; (iv) H227T, H227S, or H227I; (v) Y228P; and (vi) L229V or L229I, in the amino acid sequence of SEQ ID NO:1. In aspects, the complex comprises a nucleic acid sequence encoding the amino acid sequence of SEQ ID NO:2. In aspects, the complex comprises a nucleic acid sequence encoding the amino acid sequence of SEQ ID NO:86. In aspects, the complex comprises a nucleic acid sequence encoding the amino acid sequence of SEQ ID NO:87.

In embodiments, the complex comprises a mutant pyrrolysyl-tRNA synthetase as described herein, including embodiments thereof; fluorosulfonyloxybenzoyl-L-lysine (FSK); and tRNA^{Pyl} as described herein, including embodiments thereof. In aspects, the tRNA^{Pyl} comprises the amino acid sequence encoded by SEQ ID NO:3. In aspects, the tRNA^{Pyl} has at least 80% sequence identity to the amino acid sequence encoded by SEQ ID NO:3. In aspects, the tRNA^{Pyl} has at least 85% sequence identity to the amino acid sequence encoded by SEQ ID NO:3. In aspects, the tRNA^{Pyl} has at least 90% sequence identity to the amino acid sequence encoded by SEQ ID NO:3. In aspects, the tRNA^{Pyl} has at least 95% sequence identity to the amino acid sequence encoded by SEQ ID NO:3.

### Compositions

The disclosure provides compositions provided herein, including embodiments and aspects thereof. In embodiments, the compositions comprise fluorosulfonyloxybenzoyl-L-lysine (FSK) having Formula (A):

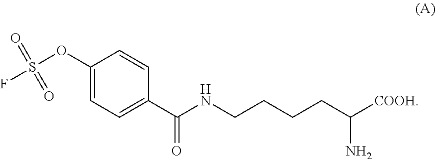 [SMILES: NC(CCCCNC(=O)c1ccc(OS(=O)(=O)F)cc1)C(=O)O]

In embodiments, the compositions further comprise components of an in vitro translation system, a variant pyrrolysyl-tRNA synthetase as described herein (including embodiments and aspects thereof), a tRNA^{Pyl} as described herein (including embodiments and aspects thereof), a vector as described herein (including embodiments and aspects thereof), a complex as described herein (including embodiments and aspects thereof), or a combination of two or more thereof.

In embodiments, the compositions comprise a variant pyrrolysyl-tRNA synthetase as described herein, including embodiments and aspects thereof. In embodiments, the compositions comprise a FSK, a tRNA^{Pyl} as described herein (including embodiments and aspects thereof), a complex as described herein (including embodiments and aspects thereof), a vector as described herein (including embodiments and aspects thereof), or a combination of two or more thereof.

In embodiments, the compositions comprise a tRNA^{Pyl} as described herein, including embodiments and aspects thereof. In embodiments, the cell further comprises FSK, a variant pyrrolysyl-tRNA synthetase as described herein (including embodiments and aspects thereof), a complex as described herein (including embodiments and aspects thereof), a vector as described herein (including embodiments and aspects thereof), or a combination of two or more thereof.

In embodiments, the compositions comprise FSK having Formula (A) and one or more compounds selected from the group consisting of tRNA (e.g., as described herein), a cofactor (e.g., initation factor, elongation factor, termination factor), an energy regenerating system (e.g., creatine phosphate and/or creatine phosphokinase for a eukaryotic system, and phosphoenol pyruvate and/or pyruvate kinase for a bacterial system), a peptide, a salt (e.g., a magnesium salt, a potassium salt), a protein, and a ribosome (e.g, 70S ribosomes, 80S ribosomes). In embodiments, the compositions comprise FSK having Formula (A) and a compound selected from the group consisting of tRNA, a cofactor, an energy regenerating system, a salt, a protein, a ribosome, and a combination of two or more thereof. In embodiments, the compositions comprise FSK having Formula (A) and a compound selected from the group consisting of a cofactor, an energy regenerating system, a salt, a ribosome, and a combination of two or more thereof. In embodiments, the compositions comprise FSK having Formula (A) and a compound selected from the group consisting of tRNA, an initation factor, an elongation factor, a termination factor, creatine phosphate, creatine phosphokinase, a magnesium salt, a potassium salt, an 80S ribosome, and a combination of two or more thereof. In embodiments, the compositions comprise FSK having Formula (A) and a compound selected from the group consisting of tRNA, an initation factor, an elongation factor, a termination factor, phosphoenol pyruvate, pyruvate kinase, a magnesium salt, a potassium salt, a 70S ribosome, and a combination of two or more thereof.

### In Vitro Translation System

In embodiments, the disclosure provides an in vitro translation system comprising a biomolecule as described herein, e.g., a biomolecule of Formula (B), Formula (C), Formula (D), Formula (E), Formula (I), Formula (II), Formula (III), including embodiments and aspects thereof. In embodiments, the in vitro translation system is a wheat germ extract in vitro translation system or a rabbit reticulocyte lystate in vitro translation system. In embodiments, the in vitro translation system is a wheat germ extract in vitro translation system. In embodiments, the in vitro translation system is a rabbit reticulocyte lystate in vitro translation system.

### Cellular Compositions

The disclosure provides cells comprising the compositions and complexes provided herein, including embodiments and aspects thereof. In embodiments, the cell comprises fluorosulfonyloxybenzoyl-L-lysine (FSK) having Formula (A):

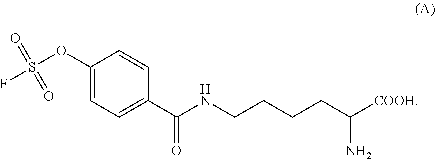 [SMILES: NC(CCCCNC(=O)c1ccc(OS(=O)(=O)F)cc1)C(=O)O]

In embodiments, the cell further comprises a variant pyrrolysyl-tRNA synthetase as described herein (including embodiments and aspects thereof), a tRNA^{Pyl} as described herein (including embodiments and aspects thereof), a vector as described herein (including embodiments and aspects thereof), a complex as described herein (including embodiments and aspects thereof), or a combination of two or more thereof.

In embodiments, the cell comprises a variant pyrrolysyl-tRNA synthetase as described herein, including embodiments and aspects thereof. In embodiments, the cell further comprises a FSK, a tRNA^{Pyl} as described herein (including embodiments and aspects thereof), a complex as described herein (including embodiments and aspects thereof), a vector as described herein (including embodiments and aspects thereof), or a combination of two or more thereof.

In embodiments, the cell comprises a tRNA^{Pyl} as described herein, including embodiments and aspects thereof. In embodiments, the cell further comprises FSK, a variant pyrrolysyl-tRNA synthetase as described herein (including embodiments and aspects thereof), a complex as described herein (including embodiments and aspects thereof), a vector as described herein (including embodiments and aspects thereof), or a combination of two or more thereof.

In aspects, the cell comprises a vector as described herein, including embodiments and aspects thereof. In aspects, the cell further comprises FSK, a variant pyrrolysyl-tRNA synthetase as described herein (including embodiments and aspects thereof), a tRNA^{Pyl} as described herein (including embodiments and aspects thereof), a complex as described herein (including embodiments and aspects thereof), or a combination of two or more thereof.

In embodiments, the cell comprises a complex as described herein, including embodiments and aspects thereof. In aspects, the cell further comprises FSK, a variant pyrrolysyl-tRNA synthetase as described herein (including embodiments and aspects thereof), a tRNA^{Pyl} as described herein (including embodiments and aspects thereof), a vector as described herein (including embodiments and aspects thereof) or a combination of two or more thereof.

In embodiments, FSK is biosynthesized inside the cell, thereby generating a cell containing FSK. In aspects, FSK is contained in the medium outside the cell and penetrates into the cell, thereby generating a cell containing FSK. In aspects, the cell comprises an FSK biomolecule. In aspects, the cell comprises an FSK protein. In aspects, the cell comprises an FSK biomolecule that is synthesized inside the cell. In aspects, the cell comprises an FSK protein that is synthesized inside the cell. In aspects, the cell comprises an FSK biomolecule that is synthesized outside a cell, and that penetrates into the cell. In aspects, the cell comprises an FSK protein that is synthesized outside a cell, and that penetrates into the cell.

In embodiments, the cell comprises the biomolecule conjugates described herein. In aspects, the cell comprises biomolecule conjugate comprising a first biomolecule moiety conjugated to a second biomolecule moiety through a bioconjugate linker, wherein the bioconjugate linker has the formula:

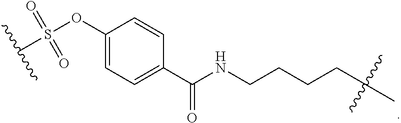 [SMILES: *CCCCNC(=O)c1ccc(OS(*)(=O)=O)cc1]

In aspects, the cell comprises a biomolecule conjugate of the formula R¹-L¹-A-X¹-L²-R², wherein the substituents are as defined herein. In aspects, the first and second biomolecule moieties are each independently a peptidyl moiety, a nucleic acid moiety, or a carbohydrate moiety. In aspects, the first and second biomolecule moieties are each a peptidyl moiety within the same protein. In aspects, the first and second biomolecule moieties are each a peptidyl moiety within different proteins (e.g., within a single-domain antibody and within a membrane receptor).

In embodiments, the cell comprises a protein which comprises a moiety of Formula (IV), a moiety of Formula (V), or a moiety of Formula (VI):

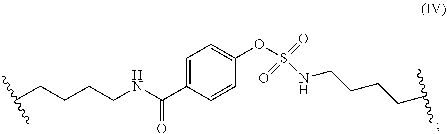 [SMILES: *CCCCNC(=O)c1ccc(OS(=O)(=O)NCCCC*)cc1]

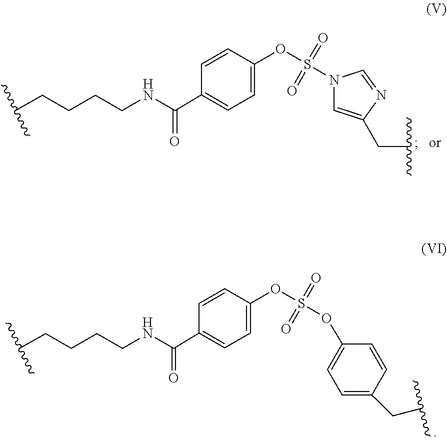 [SMILES: *CCCCNC(=O)c1ccc(OS(=O)(=O)n2cnc(C*)c2)cc1 ; *CCCCNC(=O)c1ccc(OS(=O)(=O)Oc2ccc(C*)cc2)cc1]

In aspects, the moiety of Formula (A), (B), or (C) forms an intramolecular covalent bond within a protein. In aspects, the moiety of Formula (A), (B), or (C) forms an intermolecular covalent bond between two proteins.

In embodiments, the cell comprises a protein of Formula (I), Formula (II), or Formula (III):

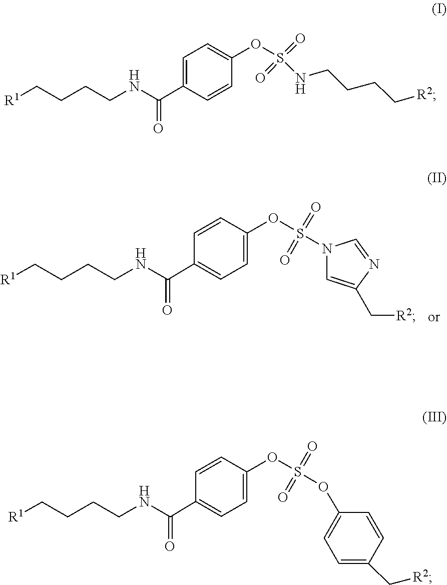

wherein R¹ and R² are each independently a peptidyl moiety. In aspects, R¹ and R² are bonded together, such that protein of Formula (I), (II), and (III) comprise an intramolecular bond. In aspects, R¹ and R² are a peptidyl moiety in two different proteins, such that the protein of Formula (I), (II), and (III) comprises an intermolecular bond between two proteins. In embodiments, R¹ is a peptidyl moiety in a single-domain antibody and R² is a peptidyl moiety in a membrane receptor. In embodiments, R¹ is a peptidyl moiety in a membrane receptor and R² is a peptidyl moiety in a single-domain antibody.

A cell can be any prokaryotic or eukaryotic cell. In aspects, the cell is prokaryotic. In aspects, the cell is eukaryotic. In aspects, the cell is a bacterial cell, a fungal cell, a plant cell, an archael cell, or an animal cell. In aspects, the animal cell is an insect cell or a mammalian cell. In aspects, the cell is a bacterial cell. In aspects, the cell is a fungal cell. In aspects, the cell is a plant cell. In aspects, the cell is an archael cell. In aspects, the cell is an animal cell. In aspects, the cell is an insect cell. In aspects, the cell is a mammalian cell. In aspects, the cell is a human cell. For example, any of the compositions described herein can be expressed in bacterial cells such as E. coli, insect cells, yeast or mammalian cells (such as Hela cells, Chinese hamster ovary cells (CHO) or COS cells). In aspects, the cell is a premature mammalian cell, i.e., a pluripotent stem cell. In aspects, the cell is derived from other human tissue. Other suitable cells are known to those skilled in the art.

### Methods of Forming a Biomolecule or Biomolecule Conjugate

The compositions provided herein are useful for forming a biomolecule or biomolecule conjugate. Thus, in an aspect is provided method of forming an FSK biomolecule by contacting a biomolecule, a mutant pyrrolysyl-tRNA synthetase, a tRNA^{Pyl}, and fluorosulfonyloxybenzoyl-L-lysine (FSK) having Formula (A):

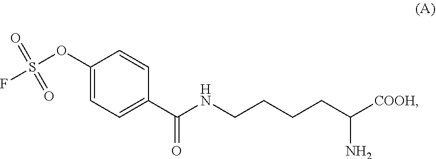 [SMILES: NC(CCCCNC(=O)c1ccc(OS(=O)(=O)F)cc1)C(=O)O]

thereby producing the FSK biomolecule, i.e., a biomolecule comprising the unnatural amino acid of FSK. The biomolecule produced by the method will comprise the unnatural amino acid side chain of Formula (F):

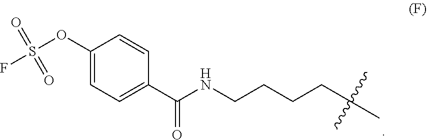 [SMILES: *CCCCNC(=O)c1ccc(OS(=O)(=O)F)cc1]

The mutant pyrrolysyl-tRNA synthetase used in the method of producing the biomolecule is any described herein. The tRNA^{Pyl} used in the method of producing the biomolecule is any described herein. In aspects, the biomolecule is a protein. In aspects, the biomolecule is a nucleic acid. In aspects, the biomolecule is a carbohydrate. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells. In aspects, the reaction is performed in one or more cells selected from the group consisting of a bacterial cell, a fungal cell, a plant cell, an archael cell, and an animal cell. In aspects, the reaction is performed in one or more cells selected from the group consisting of a bacterial cell, a fungal cell, a plant cell, an archael cell, an insect cell, and a mammalian cell.

In embodiments, the disclosure provides methods for producing an FSK protein by contacting a protein, a mutant pyrrolysyl-tRNA synthetase, a tRNA^{Pyl}, and fluorosulfonyloxybenzoyl-L-lysine (FSK) having Formula (A):

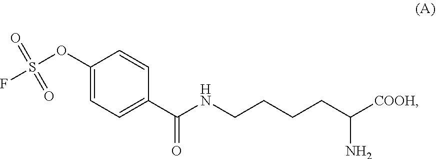 [SMILES: NC(CCCCNC(=O)c1ccc(OS(=O)(=O)F)cc1)C(=O)O]

thereby producing the FSK protein, i.e., a protein comprising the unnatural amino acid of FSK. The protein produced by the method will comprise the unnatural amino acid side chain of Formula (F):

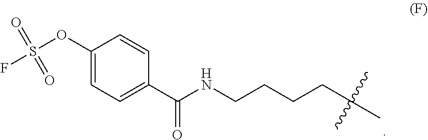 [SMILES: *CCCCNC(=O)c1ccc(OS(=O)(=O)F)cc1]

The mutant pyrrolysyl-tRNA synthetase used in the method of producing the protein is any described herein. The tRNA^{Pyl} used in the method of producing the protein is any described herein. In aspects, the FSK protein further comprises lysine, histidine, tyrosine, or two or more thereof. In aspects, the FSK protein comprises FSK that is proximal to lysine, histidine, tyrosine, or two or more thereof. In aspects, the FSK protein comprises FSK that is proximal to lysine. In aspects, the FSK protein comprises FSK that is proximal to histidine. In aspects, the FSK protein comprises FSK that is proximal to tyrosine. The term “proximal” is described herein. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells. In aspects, the reaction is performed in one or more cells selected from the group consisting of a bacterial cell, a fungal cell, a plant cell, an archael cell, and an animal cell. In aspects, the reaction is performed in one or more cells selected from the group consisting of a bacterial cell, a fungal cell, a plant cell, an archael cell, an insect cell, and a mammalian cell.

In embodiments, the disclosure provides methods for forming a biomolecule conjugate by contacting a first biomolecule moiety which comprises FSK with a second biomolecule moiety, wherein the second biomolecule moiety is reactive with the FSK in the first biomolecule moiety, thereby forming a biomolecule conjugate. In aspects, the first biomolecule moiety which comprises FSK is a compound of Formula (B):

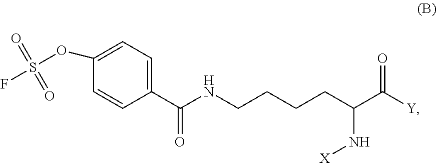

where X and Y are as defined herein. In aspects, the first biomolecule moiety which comprises FSK is a compound of Formula (C):

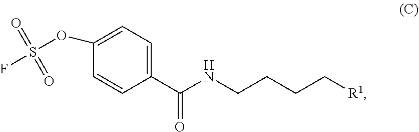

where R¹ is as defined herein. In aspects, the first biomolecule moiety which comprises FSK is a biomolecule having an amino acid side chain of Formula (F):

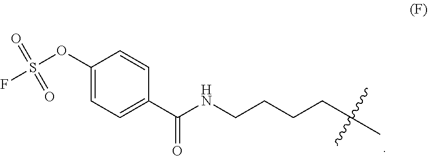 [SMILES: *CCCCNC(=O)c1ccc(OS(=O)(=O)F)cc1]

In aspects, the second biomolecule moiety comprises a lysine, histidine, or tyrosine that is reactive with the FSK in the first biomolecule. In aspects, the reaction to form the biomolecule conjugate occurs by proximity-enabled, click chemistry (e.g., between the FSK on the first biomolecule moiety and the lysine, histidine, or tyrosine on the second biomolecule moiety). In aspects, the reaction to form the biomolecule conjugate occurs by a sulfur-fluoride exchange reaction (e.g., between the FSK on the first biomolecule moiety and the lysine, histidine, or tyrosine on the second biomolecule moiety). In aspects, the reaction to form biomolecule conjugate occurs by a proximity-enabled, sulfur-fluoride exchange reaction (e.g., between the FSK on the first biomolecule moiety and the lysine, histidine, or tyrosine on the second biomolecule moiety). In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells. In aspects, the reaction is performed in one or more cells selected from the group consisting of a bacterial cell, a fungal cell, a plant cell, an archael cell, and an animal cell. In aspects, the reaction is performed in one or more cells selected from the group consisting of a bacterial cell, a fungal cell, a plant cell, an archael cell, an insect cell, and a mammalian cell.

In embodiments, the disclosure provides proteins comprising one or more intramolecular covalent bonds (e.g., a protein conjugate). In aspects, FSK and the proximal lysine, histidine, or tyrosine undergo a reaction to form the intramolecular covalent bond, resulting in a moiety of Formula (IV), a moiety of Formula (V), or a moiety of Formula (VI), or a combination of two or more thereof.

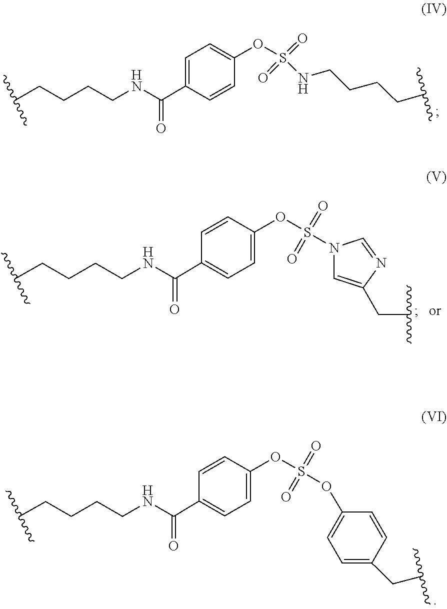 [SMILES: *CCCCNC(=O)c1ccc(OS(=O)(=O)NCCCC*)cc1 ; *CCCCNC(=O)c1ccc(OS(=O)(=O)n2cnc(C*)c2)cc1 ; *CCCCNC(=O)c1ccc(OS(=O)(=O)Oc2ccc(C*)cc2)cc1]

The FSK and the lysine, histidine, or tyrosine that are proximal thereto can be on an α-strand of the protein and/or a β-strand of the protein. In aspects, the reaction to form the intramolecular covalent bond between FSK and the lysine, histidine, or tyrosine is accomplished through click chemistry. In aspects, the reaction to form the intramolecular covalent bond between FSK and the lysine, histidine, or tyrosine is accomplished through proximity-enabled, click chemistry. In aspects, the reaction to form the intramolecular covalent bond between FSK and the lysine, histidine, or tyrosine is accomplished through a sulfur-fluoride exchange reaction. In aspects, the reaction to form the intramolecular covalent bond between FSK and the lysine, histidine, or tyrosine is accomplished through a proximity-enabled, sulfur-fluoride exchange reaction. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells. In aspects, the reaction is performed in one or more cells selected from the group consisting of a bacterial cell, a fungal cell, a plant cell, an archael cell, and an animal cell. In aspects, the reaction is performed in one or more cells selected from the group consisting of a bacterial cell, a fungal cell, a plant cell, an archael cell, an insect cell, and a mammalian cell.

In embodiments, the disclosure provides protein conjugates of Formula (I), (II), or (III) wherein R¹ and R² are each independently a peptidyl moiety:

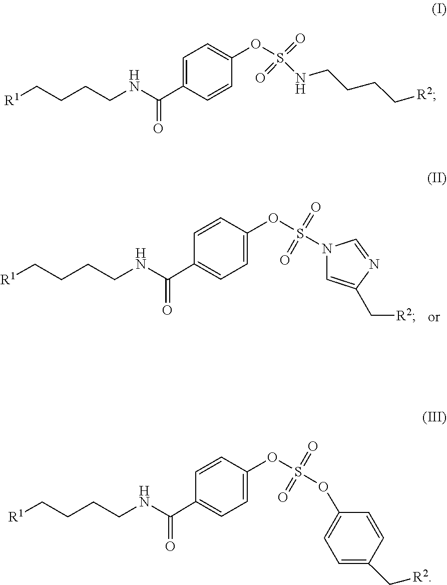

In aspects, R¹ and R² are joined together to form an intramolecularly conjugated protein. In aspects, R¹ and R² are not joined together. In aspects, the reaction to form the protein conjugates is accomplished through click chemistry. In aspects, the reaction to form the protein conjugate is accomplished through proximity-enabled, click chemistry. In aspects, the reaction to form the protein conjugate is accomplished through a sulfur-fluoride exchange reaction. In aspects, the reaction to form the protein conjugate is accomplished through a proximity-enabled, sulfur-fluoride exchange reaction. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells. In aspects, the reaction is performed in one or more cells selected from the group consisting of a bacterial cell, a fungal cell, a plant cell, an archael cell, and an animal cell. In aspects, the reaction is performed in one or more cells selected from the group consisting of a bacterial cell, a fungal cell, a plant cell, an archael cell, an insect cell, and a mammalian cell.

In embodiments, two or more proteins can be covalently linked by the methods and compositions described herein. In aspects, FSK is an unnatural amino acid in a first protein and lysine, histidine, or tyrosine are amino acids in a second protein, wherein the first protein and the second protein are different. The FSK in the first protein undergoes a reaction with the lysine, histidine, or tyrosine in the second protein to form an intermolecular covalent bond between the first and second proteins. The intermolecular covalent bond linking the two proteins is represented by a moiety of Formula (IV), moiety of Formula (V), moiety of Formula (VI), or a combination of two or more thereof:

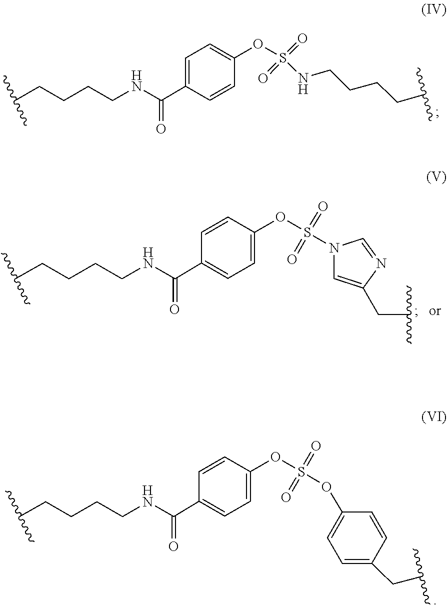 [SMILES: *CCCCNC(=O)c1ccc(OS(=O)(=O)NCCCC*)cc1 ; *CCCCNC(=O)c1ccc(OS(=O)(=O)n2cnc(C*)c2)cc1 ; *CCCCNC(=O)c1ccc(OS(=O)(=O)Oc2ccc(C*)cc2)cc1]

The FSK and the lysine, histidine, or tyrosine can be on an α-strand of their respective proteins and/or a β-strand of their respective proteins. In aspects, the reaction to form the intermolecular covalent bond between FSK in the first protein and the lysine, histidine, or tyrosine in the second protein is accomplished through click chemistry. In aspects, the reaction to form the intermolecular covalent bond between FSK in the first protein and the lysine, histidine, or tyrosine in the second protein is accomplished through proximity-enabled, click chemistry. In aspects, the reaction to form the intermolecular covalent bond between FSK in the first protein and the lysine, histidine, or tyrosine in the second protein is accomplished through sulfur-fluoride exchange. In aspects, the reaction to form the intermolecular covalent bond between FSK in the first protein and the lysine, histidine, or tyrosine in the second protein is accomplished through proximity-enabled, sulfur-fluoride exchange. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells. In aspects, the reaction is performed in one or more cells selected from the group consisting of a bacterial cell, a fungal cell, a plant cell, an archael cell, and an animal cell. In aspects, the reaction is performed in one or more cells selected from the group consisting of a bacterial cell, a fungal cell, a plant cell, an archael cell, an insect cell, and a mammalian cell.

In embodiments, the disclosure provides biomolecule conjugates comprising a first biomolecule moiety conjugated to a second biomolecule moiety through a bioconjugate linker, wherein the bioconjugate linker has Formula (D):

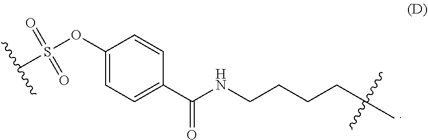 [SMILES: *CCCCNC(=O)c1ccc(OS(*)(=O)=O)cc1]

In aspects, the biomolecule conjugate has Formula (E):

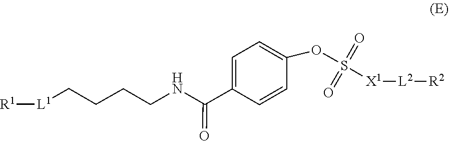

or the biomolecule conjugate has the formula R¹-L¹-A-X¹-L²-R², where the substituents are as defined herein. In aspects, the reaction to form the biomolecule conjugates is accomplished through click chemistry. In aspects, the reaction to form the biomolecule conjugate is accomplished through proximity-enabled, click chemistry. In aspects, the reaction to form the biomolecule conjugate is accomplished through a sulfur-fluoride exchange reaction. In aspects, the reaction to form the biomolecule conjugate is accomplished through a proximity-enabled, sulfur-fluoride exchange reaction. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells. In aspects, the reaction is performed in one or more cells selected from the group consisting of a bacterial cell, a fungal cell, a plant cell, an archael cell, and an animal cell. In aspects, the reaction is performed in one or more cells selected from the group consisting of a bacterial cell, a fungal cell, a plant cell, an archael cell, an insect cell, and a mammalian cell.

### Methods of Binding a Target

Provided herein are biomolecules having the structure of Formula (C):

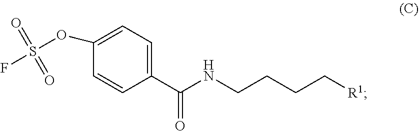

wherein R¹ is a small molecule moiety, an amino acid moiety, or a peptidyl moiety. In embodiments, R¹ is a small molecule moiety. In embodiments, R¹ is an amino acid moiety or a peptidyl moiety. In embodiments, R¹ is an amino acid moiety. In embodiments, R¹ is a peptidyl moiety. In embodiments, R¹ is an antibody, an antigen-binding fragment, a single-chain variable fragment, a single-domain antibody, or an affibody. In embodiments, R¹ is an antibody. In embodiments, R¹ is an antigen-binding fragment. In embodiments, R¹ is a single-chain variable fragment. In embodiments, R¹ is a single-domain antibody. In embodiments, R¹ is an affibody. In embodiments, R¹ is capable of binding to a target. In embodiments, R¹ is capable of binding to a target on a surface of a cell. In embodiments, the target on the surface of the cell is a receptor. In embodiments, the receptor is a membrane receptor or a hormone receptor.

In embodiments, the target is a receptor selected from the group acetylcholine receptor, an adenosine receptor, an angiotensin receptor, an apelin receptor, a bile acid, receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled estrogen receptor, a histamine receptor, a hydroxycarboxylic acid receptor, a kisspeptin receptor, a leukotriene receptor, a lysophospholipid receptor, a lysophospholipid SIP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, and a vasopressin receptor. In embodiments, the target is PD-1 or PD-L1. In embodiments, the target is PD-1. In embodiments, the target is PD-L1. In embodiments, the target is a protein, a nucleic acid, or a carbohydrate. In embodiments, the target is a protein. In embodiments, the target is a nucleic acid. In embodiments, the target is a carbohydrate.

Provided herein are methods of binding a target on a cell comprising contacting the cell with the biomolecule of Formula (B) or the biomolecule of Formula (C), wherein the biomolecule is capable of specifically binding to the target on the surface of the cell, whereby the biomolecule forms a covalent bond with the target. In embodiments, the method comprises contacting the cell with the biomolecule of Formula (B), wherein the biomolecule is capable of specifically binding to the target on the surface of the cell, whereby the biomolecule forms a covalent bond with the target. In embodiments, the method comprises contacting the cell with the biomolecule of Formula (C), wherein the biomolecule is capable of specifically binding to the target on the surface of the cell, whereby the biomolecule forms a covalent bond with the target. In embodiments, the covalent bond is formed through a sulfur-fluoride exchange reaction.

In embodiments, the covalent bond is formed through a proximity-enabled, sulfur-fluoride exchange reaction. In embodiments, biomolecule and the target are covalently linked by a bioconjugate linker having the structure of Formula (D)

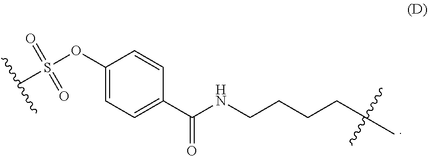 [SMILES: *CCCCNC(=O)c1ccc(OS(*)(=O)=O)cc1]

“Target” refers to any compound which is capable of binding covalently or non-covalently with R¹ (e.g., a protein). In embodiments, a “target” comprises, without limitation, small molecules, peptides, proteins, enzymes, antibodies, antigens, lipids, metabolites, hormones, carbohydrates, nucleic acids, cells, receptors, viruses, or any other moiety which is capable of binding covalently or non-covalently with R¹. In embodiments, Both R¹ and the amino acid side chain thereof (i.e., Formula (F)) can bind the target. Without intending to be bound by any theory of the invention, and for exemplary purposes only, for proximity-induced coupling, R¹ may engage the target first through non-covalent binding, followed by covalent binding through the FSK amino acid side chain.

### Embodiments 1 to 57

Embodiment 1. A biomolecule conjugate comprising a first biomolecule moiety conjugated to a second biomolecule moiety through a bioconjugate linker, wherein the bioconjugate linker has the formula:

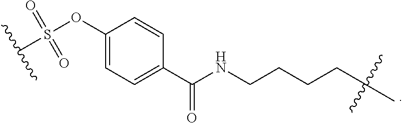 [SMILES: *CCCCNC(=O)c1ccc(OS(*)(=O)=O)cc1]

Embodiment 2. The biomolecule conjugate of Embodiment 1, wherein the biomolecule conjugate has the formula: R¹-L¹-A-X¹-L²-R²; wherein: A is the bioconjugate linker; R¹ is the first biomolecule moiety; R² is the second biomolecule moiety; L¹ is a bond or a first covalent linker; L² is a bond or a second covalent linker; and X¹ is —NR⁵—, —O—, —S—, or

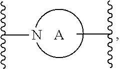

wherein ring A is a substituted or unsubstituted heteroarylene or substituted or unsubstituted heterocycloalkylene, and wherein the nitrogen in A is attached to the bioconjugate linker; and R⁵ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R¹ and R² are optionally joined together to form an intramolecularly conjugated biomolecule conjugate.

Embodiment 3. The biomolecule conjugate of Embodiment 2, wherein L¹ is a bond, —S(O)₂—, —NR^{3A}—, —O—, —S—, —C(O)—, —C(O)NR^{3A}—, —NR^{3A}C(O)—, —NR^{3A}C(O)NR^{3B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L² is a bond, —S(O)₂—, —NR^{4A}—, —O—, —S—, —C(O)—, —C(O)NR^{4A}—, —NR^{4A}C(O)—, —NR^{4A}C(O)NR^{4B}—, —C(O)O—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R^{3A}, R^{3B}, R^{4A}, and R^{4B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 4. The biomolecule conjugate of Embodiment 2 or 3, wherein X¹ is —NH—, —O—, or imidazolylene.

Embodiment 5. The biomolecule conjugate of any one of Embodiments 1 to 4, wherein the first biomolecule moiety is a peptidyl moiety, a nucleic acid moiety, or a carbohydrate moiety.

Embodiment 6. The biomolecule conjugate of Embodiment 5, wherein the first biomolecule moiety is a peptidyl moiety; and wherein the peptidyl moiety is covalently bonded to the bioconjugate linker via lysine, histidine, or tyrosine.

Embodiment 7. The biomolecule conjugate of any one of Embodiments 1 to 4, wherein the second biomolecule moiety is a peptidyl moiety, a nucleic acid moiety, or a carbohydrate moiety.

Embodiment 8. The biomolecule conjugate of Embodiment 7, wherein the second biomolecule moiety is a peptidyl moiety; and wherein the peptidyl moiety is covalently bonded to the bioconjugate linker via lysine, histidine, or tyrosine.

Embodiment 9. The biomolecule conjugate of any one of Embodiments 2 to 4, wherein -L¹-R¹ is a peptidyl moiety, a nucleic acid moiety, or a carbohydrate moiety.

Embodiment 10. The biomolecule conjugate of any one of Embodiments 2 to 4, wherein -L²-R² is a peptidyl moiety, a nucleic acid moiety, or a carbohydrate moiety.

Embodiment 11. The biomolecule of any one of Embodiments 5 to 10, wherein the peptidyl moiety comprises a single-domain antibody or a membrane receptor.

Embodiment 12. The biomolecule of any one of Embodiments 1 to 4, wherein the peptidyl moiety in R¹ comprises a single-domain antibody and the peptidyl moiety in R² comprises a membrane receptor; or wherein the peptidyl moiety in R¹ comprises a membrane receptor and the peptidyl moiety in R² comprises a single-domain antibody.

Embodiment 13. The biomolecule conjugate of any one of Embodiments 1 to 11, wherein the bioconjugate linker is an intermolecular linker.

Embodiment 14. The biomolecule conjugate of any one of Embodiments 1 to 11, wherein the bioconjugate linker is an intramolecular linker.

Embodiment 15. A protein of Formula (I), Formula (II), or Formula (III):

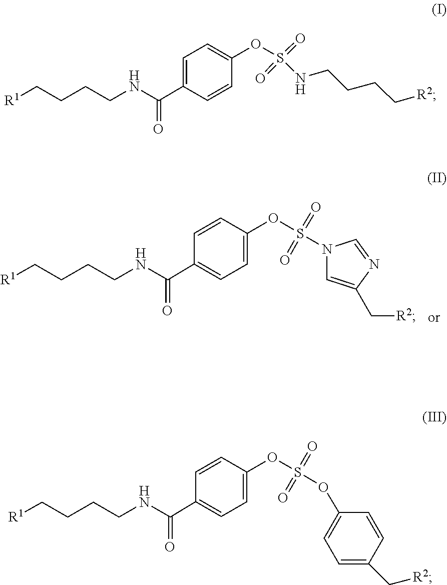

wherein R¹ and R² are each independently a peptidyl moiety; and wherein R¹ and R² are optionally joined together to form an intramolecularly conjugated protein.

Embodiment 16. The protein of Embodiment 15, wherein the protein is of Formula (I).

Embodiment 17. The protein of Embodiment 15, wherein the protein is of Formula (II).

Embodiment 18. The protein of Embodiment 15, wherein the protein is of Formula (III).

Embodiment 19. The protein of any one of Embodiments 15 to 18, wherein R¹ and R² each independently comprise a protein α-strand or a protein β-strand.

Embodiment 20. The protein of any one of Embodiments 15 to 19, wherein R¹ and R² are not joined together.

Embodiment 21. The protein of any one of Embodiments 15 to 20, wherein the peptidyl moiety of R¹ comprises a single-domain antibody and the peptidyl moiety of R² comprises a membrane receptor.

Embodiment 22. The protein of any one of Embodiments 15 to 20, wherein the peptidyl moiety of R¹ comprises a membrane receptor and the peptidyl moiety of R² comprises a single-domain antibody.

Embodiment 23. The protein of any one of Embodiments 15 to 19, wherein R¹ and R² are joined together to form an intramolecularly conjugated protein.

Embodiment 24. A pyrrolysyl-tRNA synthetase comprising at least 6 amino acid residues substitutions within the substrate-binding site of the pyrrolysyl-tRNA synthetase having the amino acid sequence of SEQ ID NO:1; wherein the substrate-binding site comprises residues tyrosine at position 126, methionine at position 129, valine at position 168, histidine at position 227, tyrosine at position 228, and lysine at position 229 as set forth in the amino acid sequence of SEQ ID NO:1.

Embodiment 25. The pyrrolysyl-tRNA synthetase of Embodiment 24, wherein the at least 6 amino acid residues substitutions in the amino acid sequence of SEQ ID NO:1 are: (i) Y126G; (ii) M129A; (iii) V168F; (iv) H227T, H227S, or H227I; (v) Y228P; and (vi) L229I, L229V, or L229I.

Embodiment 26. The pyrrolysyl-tRNA synthetase of Embodiment 24, comprising an amino acid sequence of SEQ ID NO:2.

Embodiment 27. A vector comprising a nucleic acid sequence encoding the pyrrolysyl-tRNA synthetase of any one of Embodiments 24 to 26.

Embodiment 28. The vector of Embodiment 27, further comprising a nucleic acid encoding tRNA^{Pyl}.

Embodiment 29. A complex comprising the pyrrolysyl-tRNA synthetase of any one of Embodiments 24 to 26 and a fluorosulfonyloxybenzoyl-L-lysine having the following formula:

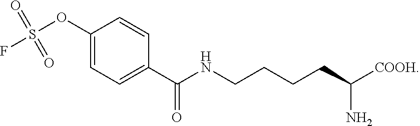 [SMILES: N[C@@H](CCCCNC(=O)c1ccc(OS(=O)(=O)F)cc1)C(=O)O]

Embodiment 30. The complex of Embodiment 29, further comprising a tRNA^{Pyl}.

Embodiment 31. A cell comprising the biomolecule conjugate of any one of Embodiments 1 to 14.

Embodiment 32. A cell comprising the protein of any one of Embodiments 15 to 23.

Embodiment 33. A cell comprising the pyrrolysyl-tRNA synthetase of any one of Embodiments 24 to 26.

Embodiment 34. A cell comprising the vector of Embodiment 27 or 28.

Embodiment 35. A cell comprising the complex of Embodiment 29 or 30.

Embodiment 36. A cell comprising fluorosulfonyloxybenzoyl-L-lysine of the formula:

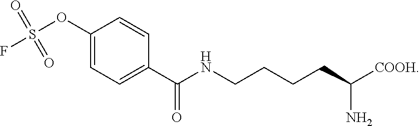 [SMILES: N[C@@H](CCCCNC(=O)c1ccc(OS(=O)(=O)F)cc1)C(=O)O]

Embodiment 37. The cell of Embodiment 36, further comprising a pyrrolysyl-tRNA synthetase comprising at least 6 amino acid residues substitutions within the substrate-binding site of the pyrrolysyl-tRNA synthetase having the amino acid sequence of SEQ ID NO:1; wherein the substrate-binding site comprises residues tyrosine at position 126, methionine at position 129, valine at position 168, histidine at position 227, tyrosine at position 228, and lysine at position 229 as set forth in the amino acid sequence of SEQ ID NO:1.

Embodiment 38. The cell of Embodiment 37, wherein the at least 6 amino acid residues substitutions in the amino acid sequence of SEQ ID NO:1 are: (i) Y126G; (ii) M129A; (iii) V168F; (iv) H227T, H227S, or H227I; (v) Y228P; and (vi) L229V or L229I.

Embodiment 39. The cell of Embodiment 37, wherein the pyrrolysyl-tRNA synthetase comprises an amino acid sequence of SEQ ID NO:2.

Embodiment 40. The cell of any one of Embodiments 36 to 39, further comprising a tRNA^{Pyl}.

Embodiment 41. The cell of any one of Embodiments 31 to 40, wherein the cell is a bacterial cell or a mammalian cell.

Embodiment 42. A method of forming the biomolecule conjugate of Embodiment 13, the method comprising contacting a fluorosulfonyloxybenzoyl-L-lysine moiety within a fluorosulfonyloxybenzoyl-L-lysine biomolecule with a compound comprising the second biomolecule moiety, wherein the second biomolecule is reactive with the fluorosulfonyloxybenzoyl-L-lysine moiety; thereby forming the biomolecule conjugate having an intermolecular linker.

Embodiment 43. A method of forming the biomolecule conjugate of Embodiment 14, the method comprising contacting a fluorosulfonyloxybenzoyl-L-lysine moiety within a fluorosulfonyloxybenzoyl-L-lysine biomolecule with a second biomolecule moiety in the fluorosulfonyloxybenzoyl-L-lysine biomolecule, wherein the second biomolecule is reactive with the fluorosulfonyloxybenzoyl-L-lysine moiety; thereby forming the biomolecule conjugate having an intramolecular linker.

Embodiment 44. The method of Embodiment 42 or 43, wherein the contacting is performed within a cell.

Embodiment 45. The method of any one of Embodiments 42 to 44, further comprising, prior to contacting, the step contacting a biomolecule, a pyrrolysyl-tRNA synthetase of any one of Embodiments 24 to 26, a tRNA^{Pyl}, and a fluorosulfonyloxybenzoyl-L-lysine having the formula:

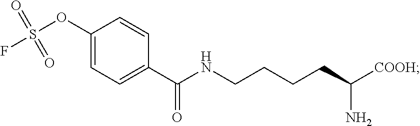 [SMILES: N[C@@H](CCCCNC(=O)c1ccc(OS(=O)(=O)F)cc1)C(=O)O]

to form the fluorosulfonyloxybenzoyl-L-lysine biomolecule.

Embodiment 46. A method of forming the protein of any one of Embodiments 20 to 22, the method comprising contacting the fluorosulfonyloxybenzoyl-L-lysine in a fluorosulfonyloxybenzoyl-L-lysine protein with a lysine, histidine, or tyrosine in a second protein; thereby forming the intermolecularly conjugate protein.

Embodiment 47. A method of forming the protein of Embodiment 23, the method comprising contacting a fluorosulfonyloxybenzoyl-L-lysine protein with a second protein comprising lysine, histidine, or tyrosine; thereby forming the intramolecularly conjugated protein.

Embodiment 48. The method of Embodiment 46 or 47, further comprising producing the fluorosulfonyloxybenzoyl-L-lysine protein, the method comprising contacting a protein, a pyrrolysyl-tRNA synthetase of any one of Embodiments 24 to 26, a tRNA^{Pyl}, and fluorosulfonyloxybenzoyl-L-lysine having the formula:

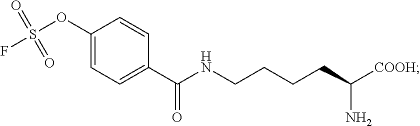 [SMILES: N[C@@H](CCCCNC(=O)c1ccc(OS(=O)(=O)F)cc1)C(=O)O]

thereby producing the fluorosulfonyloxybenzoyl-L-lysine protein.

Embodiment 49. The method of Embodiment 48, wherein contacting comprises a sulfur-fluoride exchange reaction.

Embodiment 50. The method of Embodiment 48, wherein contacting comprises a proximity-enabled, sulfur-fluoride exchange reaction.

Embodiment 51. The method of any one of Embodiments 46 to 50, wherein contacting is performed within a cell.

Embodiment 52. A protein comprising an unnatural amino acid; wherein the unnatural amino acid has a side chain of formula:

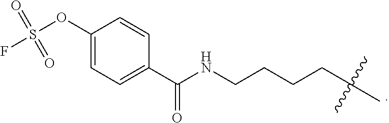 [SMILES: *CCCCNC(=O)c1ccc(OS(=O)(=O)F)cc1]

Embodiment 53. The protein of Embodiment 52, wherein the protein is a single-domain antibody.

Embodiment 54. The protein of Embodiment 52, wherein the protein is a membrane receptor.

Embodiment 55. The protein of any one of Embodiments 52 to 54, wherein the unnatural amino acid is proximal to a lysine, a histidine, or a tyrosine.

Embodiment 56. A protein comprising a moiety of Formula (IV), a moiety of Formula (V), a moiety of Formula (VI), or a combination of two or more thereof:

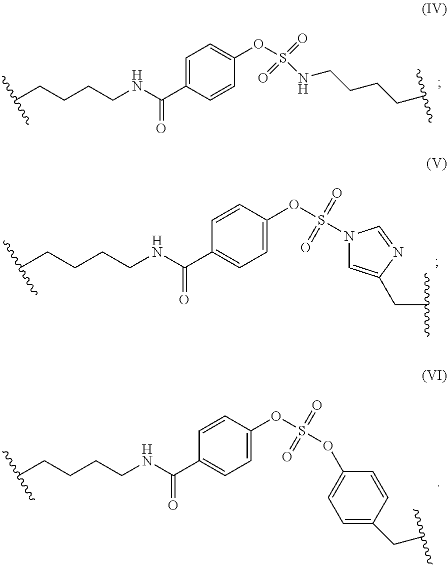 [SMILES: *CCCCNC(=O)c1ccc(OS(=O)(=O)NCCCC*)cc1 ; *CCCCNC(=O)c1ccc(OS(=O)(=O)n2cnc(C*)c2)cc1 ; *CCCCNC(=O)c1ccc(OS(=O)(=O)Oc2ccc(C*)cc2)cc1]

Embodiment 57. A cell comprising the protein of any one of Embodiments 52 to 56.

To further expand the scope of the genetically encoded SuFEx click chemistry for proteins, the inventors designed a new latent bioreactive Uaa fluorosulfonyloxybenzoyl-L-lysine (FSK) and evolved a new synthetase to genetically encode it in E. coli and mammalian cells. As a lysine derivative bearing aryl fluorosulfate, FSK offers a larger reaction distance and is more flexible than FSY. The inventors demonstrated that FSK is useful in generating covalent bonds to connect protein sites separated in long distances unreachable with FSY, both intra- and inter-molecularly, and compatible for use in vitro and in cells. FSK complements FSY enabling the introduction of covalent bonds via SuFEx chemistry into a broader range of protein sites for general applications.

### EXAMPLES

The following examples are intended to further illustrate certain embodiments and aspects of the disclosure. The examples are not intended to limit the spirit or scope of the disclosure or claims.

### Example 1

Protein side chains can spontaneously form a covalent linkage via cysteines only. This natural barrier has been broken by adding into proteins new covalent bonds formed between a genetically encoded latent bioreactive unnatural amino acid (Uaa) and a nearby natural residue via proximity-enabled reactivity. (Ref: 1, 2). A collection of bioreactive Uaas containing halogen, acrylamide, vinyl sulfone, aryl carbamate, fluorosulfate, or quinone methide have been genetically encoded to target Cys, Lys, His, Tyr, and other nucleophilic residues. (Refs: 3-8). These new covalent bonds have been engineered into proteins to enhance optical, thermal, and various protein properties, as well as to photo-modulate protein structure and function. (Refs: 1, 3, 9-11). The covalent linkage can also form between proteins, which has been exploited to capture weak and transient protein interactions for identification. (Refs: 12, 13).

Among the bioreactive functional groups, fluorosulfate is of particular interest for its exceptional biocompatibility, proximity-dependent reactivity, and multi-targeting ability. (Refs: 14-17). It is an excellent latent group which doesn't react with non-interacting proteins randomly, but react efficiently with nucleophilic residues including His, Lys, Tyr only when they are located in close proximity. (Ref. 7). The inventors recently genetically encode fluorosulfate-L-tyrosine (FSY) and demonstrated its use for not only protein cross-linkings but also generating covalent protein drugs for in vivo cancer. (Refs: 7, 18). Nonetheless, as a tyrosine derivative, FSY has a relatively rigid side chain and limited reaction radius, which will not be able to crosslink a target residue located further away in space. To maximize the capability of using fluorosulfate for generating covalent bonds for proteins, here the inventors designed and genetically encoded fluorosulfonyloxybenzoyl-L-lysine (FSK) which bears a long aliphatic side chain offering greater flexibility and longer reaction distance than FSY. We showed that FSK enabled covalent bonding within and between proteins wherein FSY fell short, and demonstrated the versatile use of FSK in various applications in vitro and in cells.

Design and Genetic Incorporation of FSK into Proteins

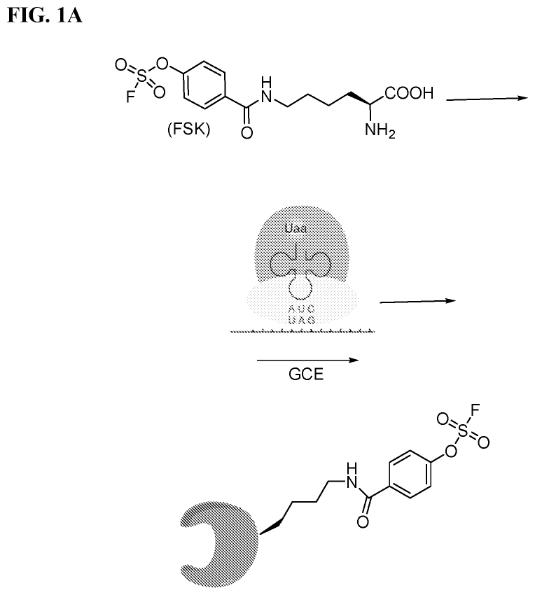

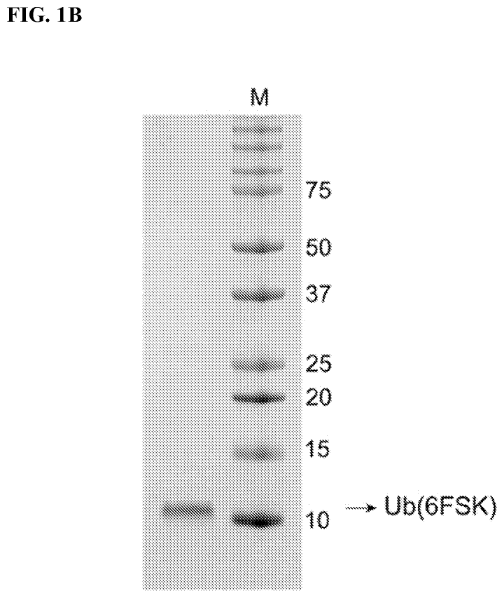

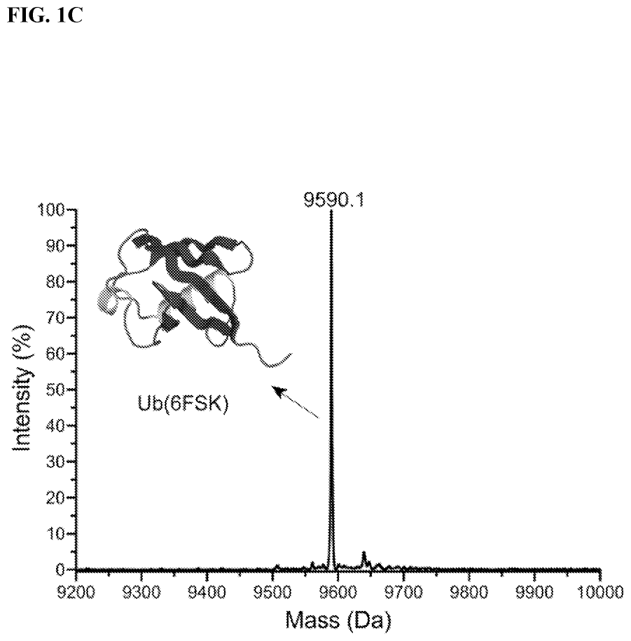

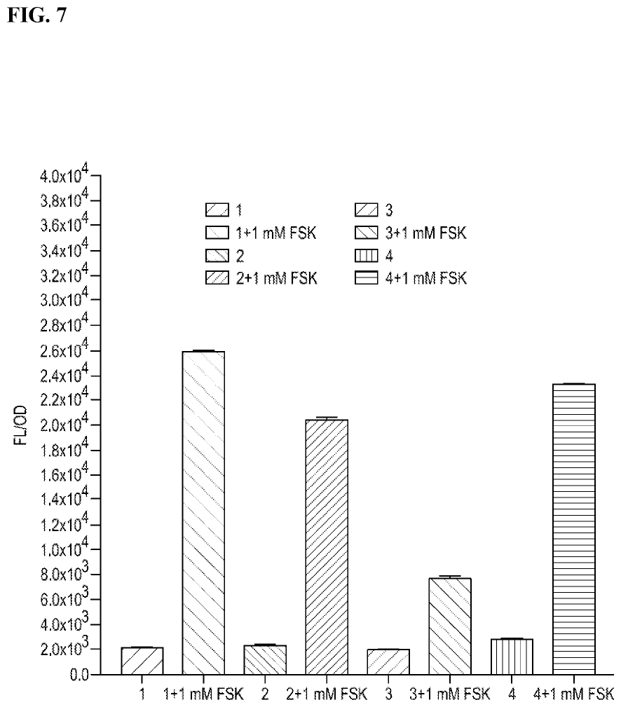

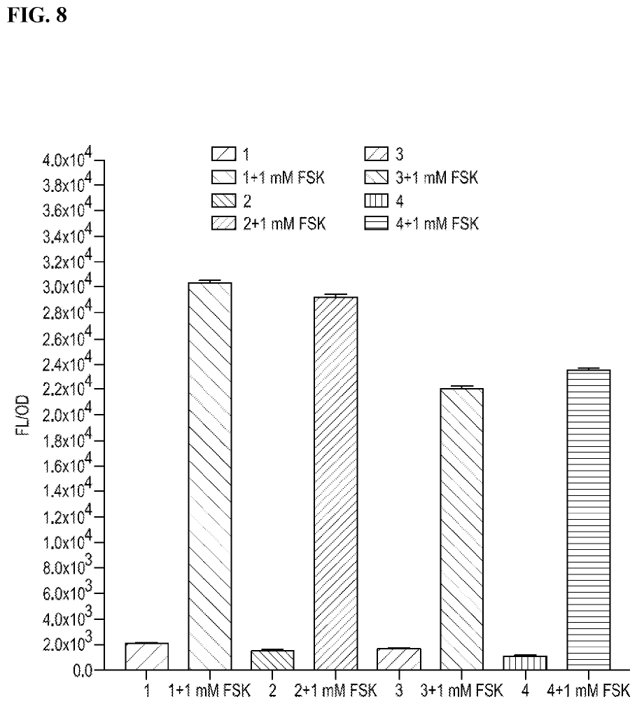

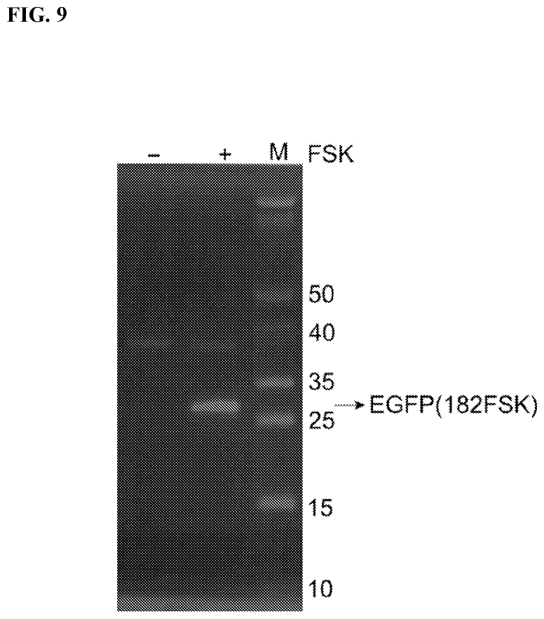

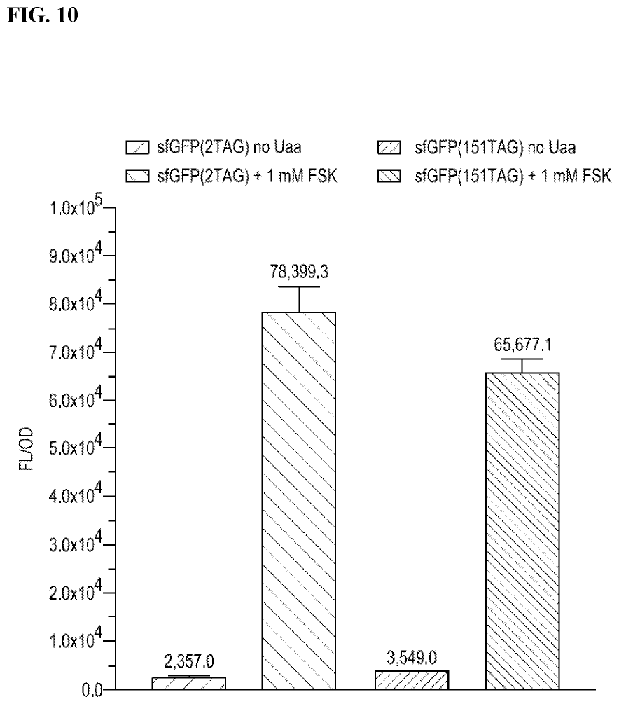

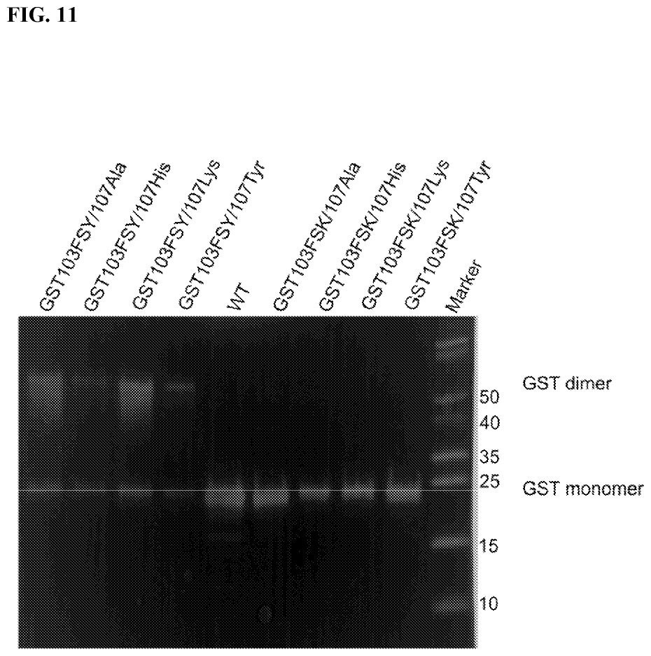

To afford flexibility and long reaction radius to fluorosulfate, we designed FSK by attaching the aryl fluorosulfate group, which is critical for the biocompatibility and SuFEx reactivity, to the Lys backbone (FIG. 1A). We then engineered an orthogonal synthetase to incorporate FSK specifically, using directed evolution strategies described previously. (Ref: 19). After several rounds of evolution, four hits were identified which could efficiently incorporate FSK into the enhanced green fluorescence protein (EGFP) rendering cells green fluorescent. Among them, hit 1 (SEQ ID NO:2) incorporated FSK into EGFP at both 18° C. and 30° C. with highest suppression efficiency, and thus was named as FSKRS (FIGS. 7-8). Western blot analysis of EGFP(182TAG) expression also showed that FSKRS only incorporate FSK and no natural amino acids (FIG. 9). The inventors also tested FSK incorporation into the superfolder GFP (sfGFP) at site 2 and site 151. At both positions, strong sfGFP fluorescence was detected when FSK was added to the growth media, in comparison to control samples without FSK (FIG. 10), confirming the specificity of FSKRS for the Uaa. To further evaluate the incorporation fidelity, the inventors incorporated FSK into a small protein ubiquitin at position 6 and measured the intact mass of the purified protein with ESI-MS. The measured molecular weight of 9590.1 Da matched well with the theoretical value (9589.9 Da), and no other peaks corresponding to natural amino acid mis-incorporation was identified, indicating the high fidelity of FSK incorporation by FSKRS (FIG. 1B).

FSK Enables Inter-Protein Crosslinking at Distance Unreachable with FSY in Cells

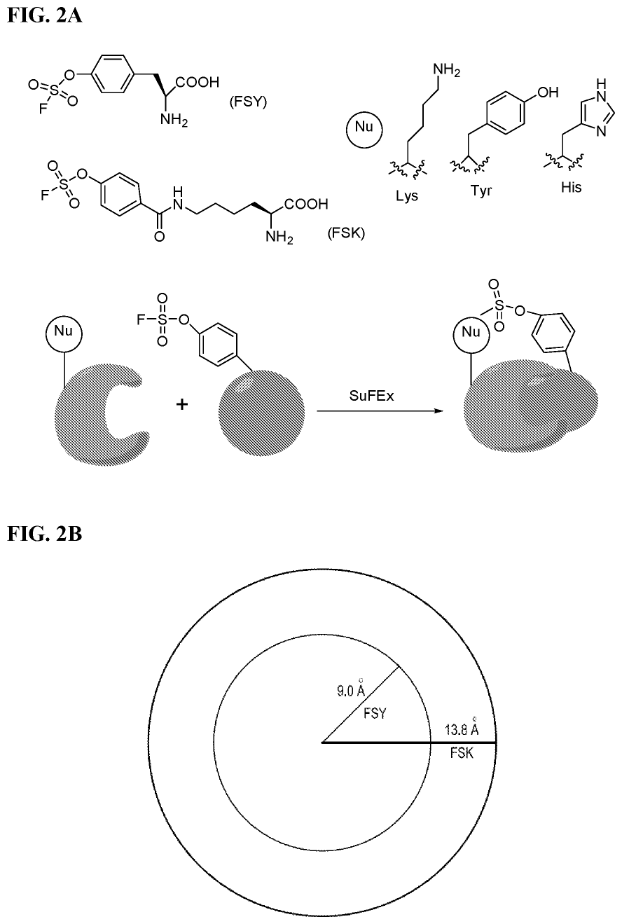

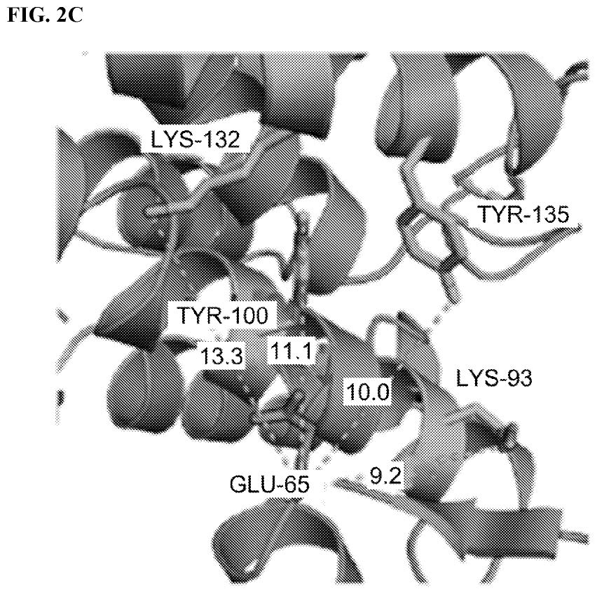

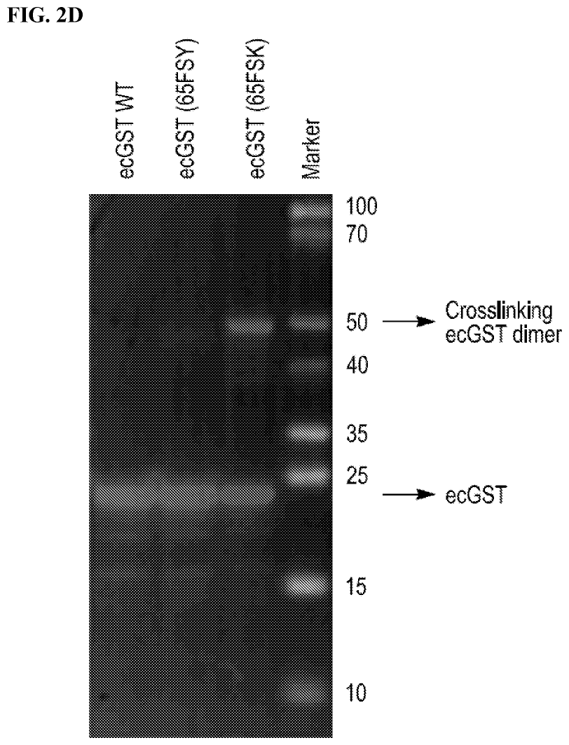

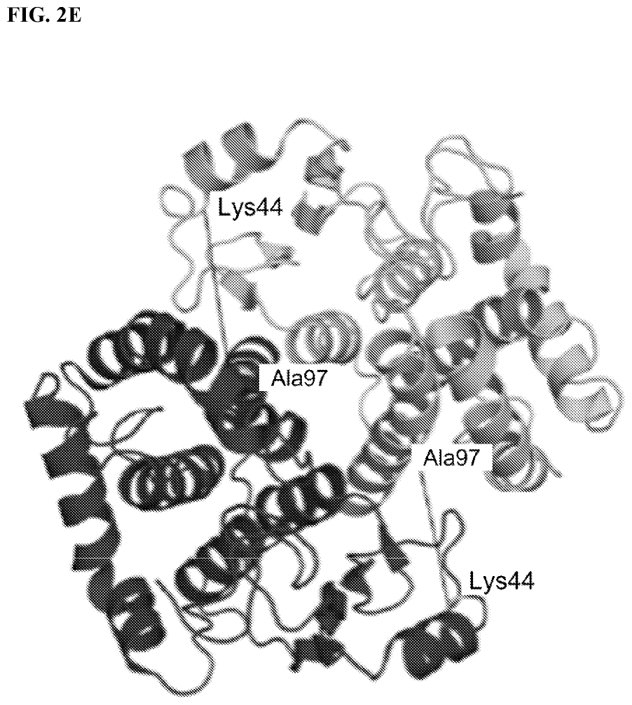

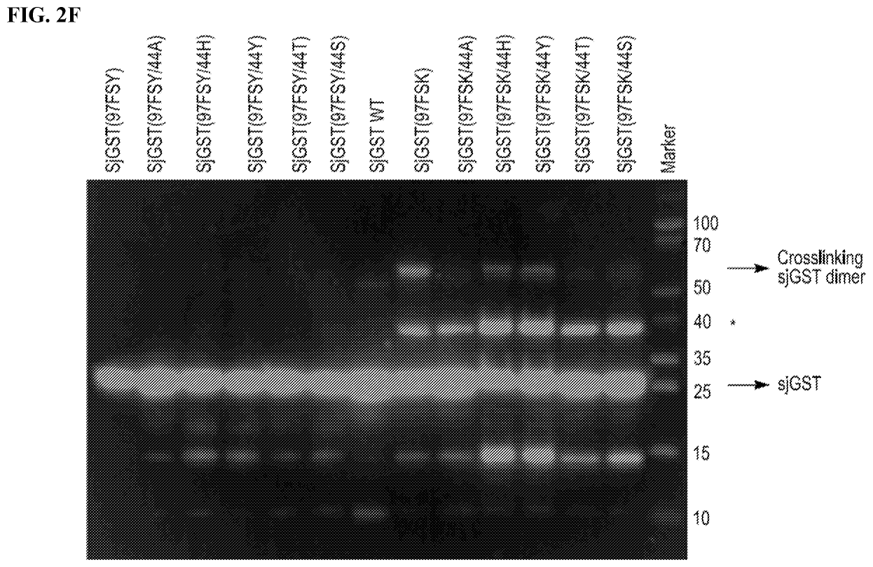

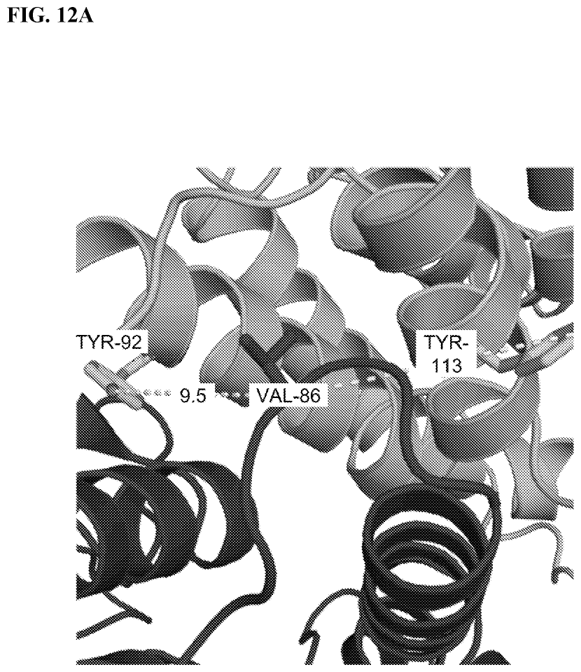

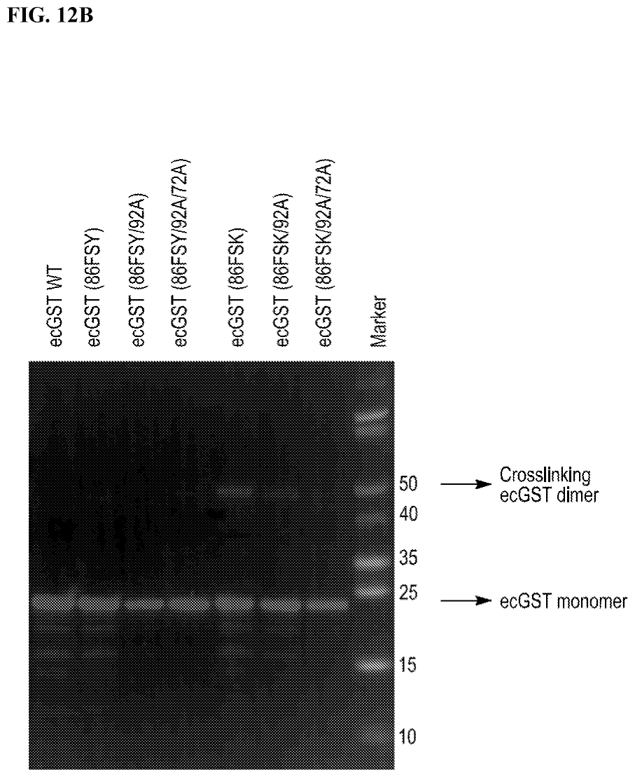

The inventors investigated the reaction distance preference for FSY and FSK when they were incorporated into proteins and reacting with targeting natural residues in proximity via SuFEx reaction (FIG. 2A). At energy minimized state, the distance between Cα and F atom was 9.0 Å for FSY and 13.8 Å for FSK (FIG. 2B). Using these lengths as a guide, the inventors tested their inter-protein crosslinking ability on the E. coli glutathione transferase (ecGST), a homodimeric protein. FSY or FSK was first incorporated into site 103 of ecGST at the dimer interface, near which His106 and Lys107 of the other monomer were potential target residues. Based on the ecGST crystal structure, the Cα of residue 103 is 8.5 Å from the δ-N atom of His106 and 6.0 Å from the ε-N atom of Lys107. (Ref 20). Strong ecGST dimeric crosslinking was found when FSY was incorporated, but no apparent dimeric crosslinking was detected when FSK was incorporated (FIG. 12), indicating that FSK was not suitable for target residue located too close in the restricted space of the dimer interface.

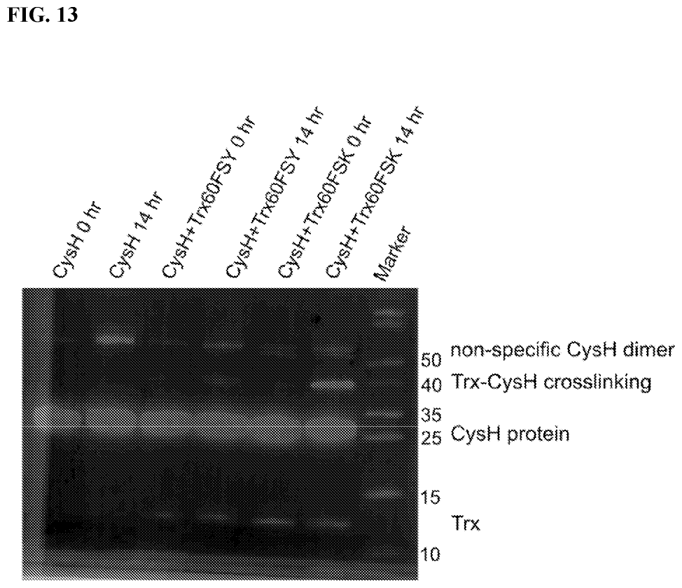

The inventors next tested the ability of FSK to crosslink target residues that were too far for FSY. The inventors chose to incorporate FSY or FSK at site 65 of ecGST, around which multiple nucleophilic residues (Lys93, Tyr100, Lys 132, Tyr 135) reside with a distance to the alpha carbon spanning from 9.2 to 13.3 Å (FIG. 2C). This distance should be favorable for FSK to react but too far for FSY. Indeed, after incorporating FSY or FSK into site 65, we found that FSK induced significant ecGST dimeric crosslinking but not FSY (FIG. 2D). The inventors also incorporated FSK or FSY into position 86 of ecGST, for which Tyr92 and Tyr72 were located 9.5 Å and 11.3 Å away, respectively (FIG. 13A). Similar results were obtained: FSK crosslinked ecGST into the dimer while FSY incorporated at the same position did not induce apparent crosslinking (FIG. 13B).

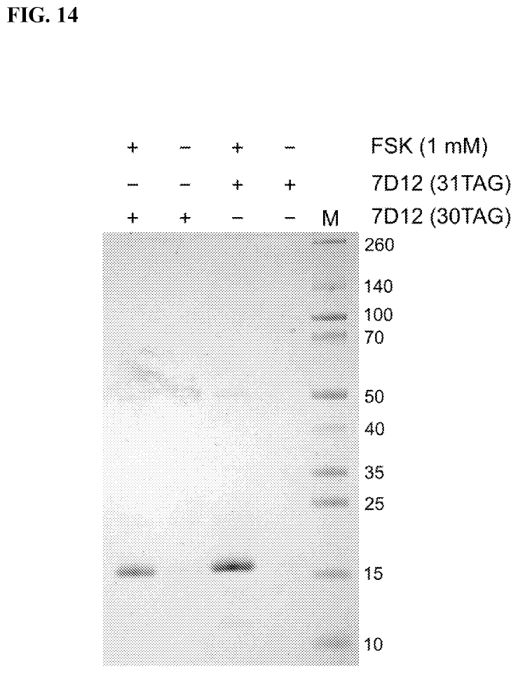

In addition to crosslinking the homodimeric ecGST, we also compared the crosslinking ability of FSY and FSK for hetero protein interaction complex. E. coli thioredoxin (Trx) interacts with 3′-phosphoadenosine-5′-phosphosulfate (PAPS) reductase. The inventors previously found that incorporating FSY at site 60 of E. coli Trx could not crosslink Trx with PAPS reductase efficiently. (Ref: 7). Examining the structure of Trx in complex with PAPS showed that the nearest possible target residue was His242 of PAPS, which was 12.2 Å away from the Cα of residue 60 of Trx. (Ref: 21). The inventors therefore tested whether incorporating FSK at the same site would improve the crosslinking efficiency due to the long flexible arm of FSK. Pleasingly, while only faint crosslinking was detectable for FSY, FSK enabled a robust crosslinking between Trx and PAPS reductase on Western blot (FIG. 14).

Next we asked what nucleophilic natural residues FSK could react with. The inventors tested its residue specificity using a residue pair Ala97 and Lys44 of sjGST protein, which has a distance of 11.7 Å between Cα of Ala97 to N$ of Lys44. (Ref: 22). The inventors generated a series of mutants by mutating Lys44 into His, Tyr, Ala, Ser, or Thr, and incorporated FSK into position 97 (FIG. 2E). Western blot analysis showed that FSK crosslinked with His, Tyr, Lys residues forming stable sjGST dimer (FIG. 2F). As previously reported, FSY also forms stable crosslinks with His, Tyr, and Lys. (Ref: 7). The consistent reactivity of FSY and FSK is expected since they both are designed to contain the same aryl fluorosulfate group. Moreover, we also incorporated FSY at site 97 into these mutants, but did not observe any apparent dimeric sjGST crosslinking (FIG. 2F), further corroborating the distance difference between FSK and FSY. Taken together, FSK possessed the same multi-targeting reaction specificity with FSY and enabled protein crosslinking at distance unreachable with FSY.

### Fsk Enables Covalent Bonding of Proteins Intramolecularly

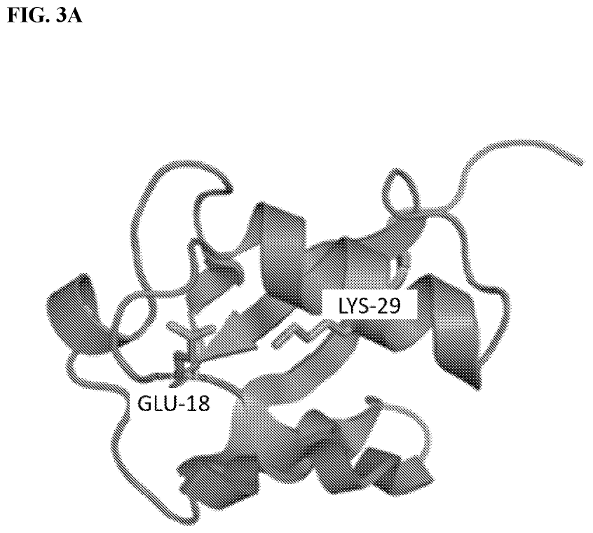

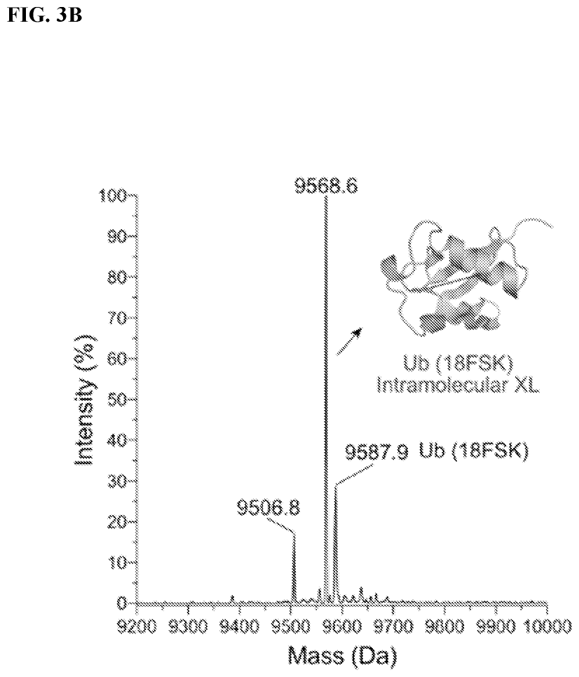

Genetically introducing intramolecular crosslinking within a peptide or protein is an innovative way to staple or bridge protein residues for engineering properties such as thermostability and cell permeability. Current methods mainly rely on disulfide bond formation between two Cys residues or targeting the thiol group of Cys with halogen or a Michael receptor installed on a bioreactive Uaa. This greatly limits the number of conformations and configurations can be created for the crosslinked peptides or proteins. Since FSK reacts with multiple nucleophilic residues that are more abundant in proteins than Cys and has favorable longer reaction arm, we reasoned that FSK would expand the diversity of crosslinking patterns for genetically encoded intramolecular crosslinks of proteins. As a proof of concept, we investigated the intramolecular crosslinking ability of FSK on a model protein ubiquitin. The inventors incorporated FSK into position 18 of ubiquitin (Ub) for its proximity with Lys29 (FIG. 3A). (Ref. 23). Mass spectrometric analysis of the intact protein showed a major peak (75% in intensity) with a mass loss of 20 Da, suggesting the successful formation of intramolecular crosslinking (FIG. 3B). To determine whether the crosslinking occurred between FSK and Lys29 as predicted from the crystal structure of Ub, we further trypsin digested the crosslinked Ub (18FSK) and analyzed with tandem mass spectrometry in high resolution. The crosslinked peptide was identified by tandem mass spec (not shown), and a series of b and y ion of the crosslinked peptide unambiguously demonstrated that FSK18 reacted with Lys29 in Ub. Besides this crosslinked peptide, we also identified the FSK-incorporated peptide (tandem mass spectrometry results not shown), which did not react with other peptides randomly, indicating the high specificity of FSK in generating intramolecular protein crosslinks

FSK and FSY Enable Covalently Targeting of EGFR Receptor with a Nanobody at Different Sites

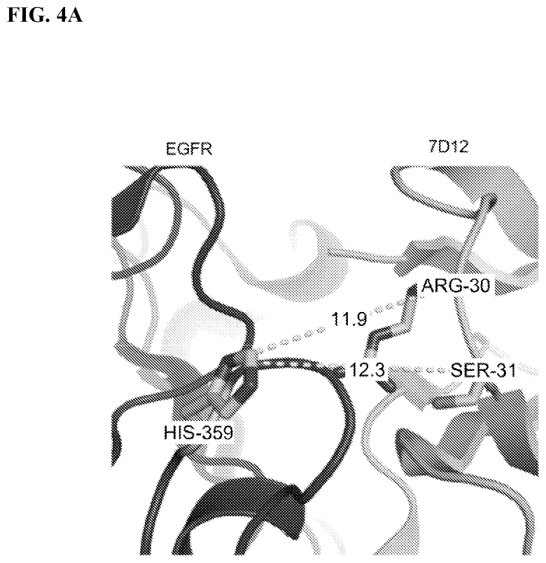

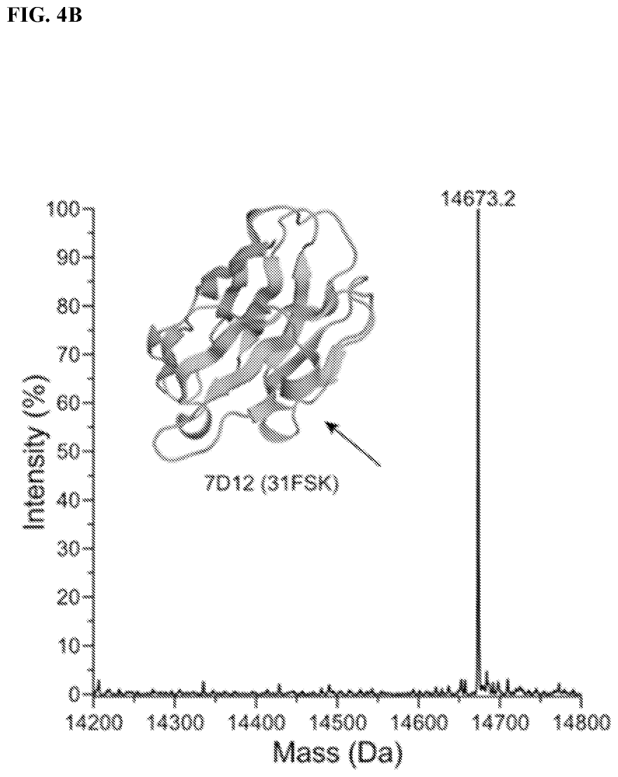

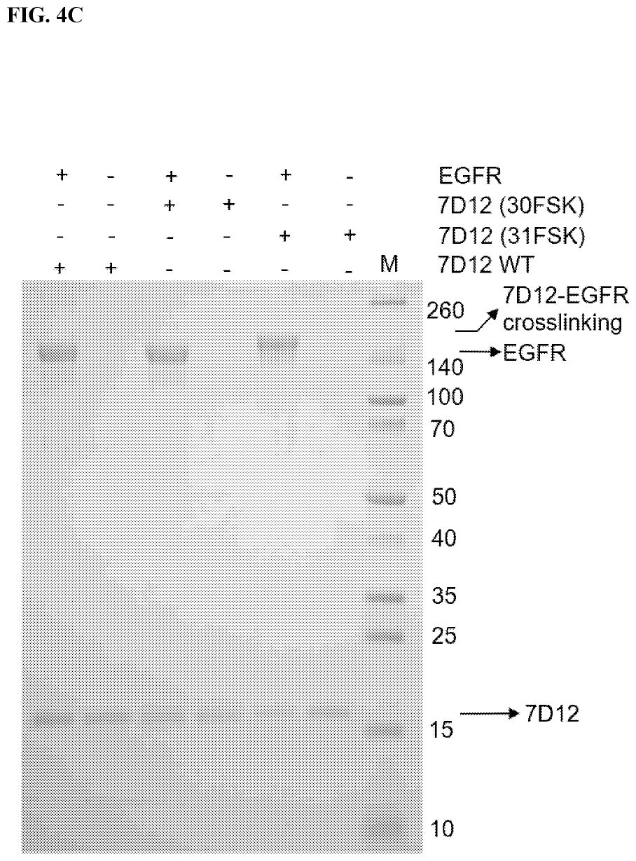

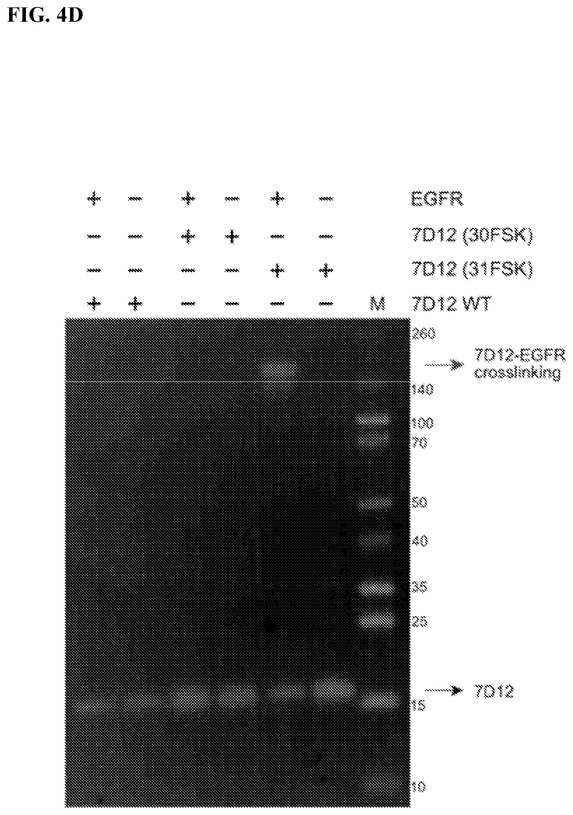

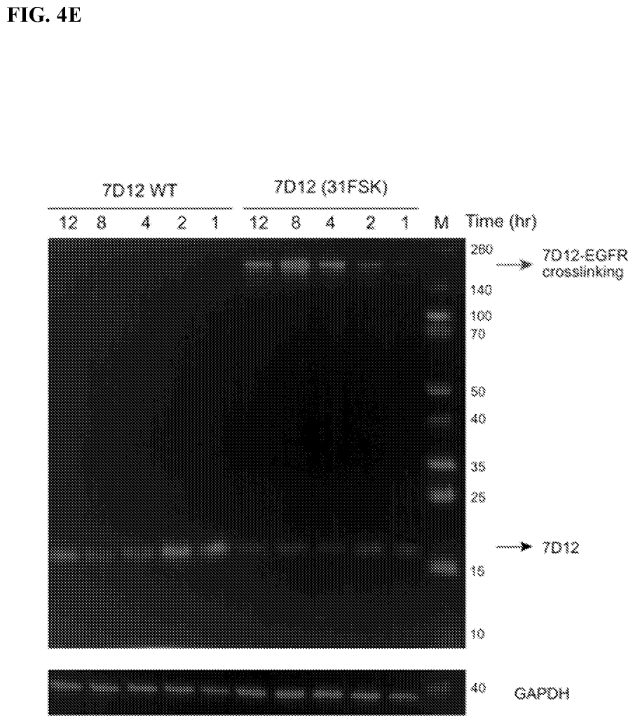

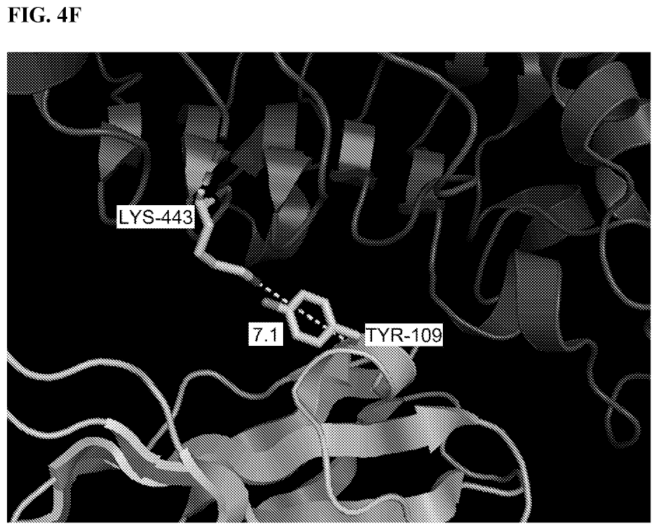

The ability to covalently target native receptors on cells and in vivo with various protein binders such as antibodies and nanobodies would afford powerful avenues for imaging, diagnostics, and therapeutics EGFR is a valuable marker for various cancers, so we aimed to covalently target it with nanobodies. (Ref: 24). Since FSK and FSY have different reaction distances, we should be able to target different sites of EGFR by incorporating FSK or FSY into the nanobody. Based on the crystal structure of nanobody 7D12 in complex with E²⁵ we predicted that incorporation of FSK into site 30 or 31 of nanobody 7D12 would potentially crosslink with His359 of EGFR, as the distance from Cα of the two sites to the δ-N atom of His359 are 11.9 and 12.3 Å, respectively (FIG. 4A). Similarly, incorporation of FSY into site 109 of nanobody 7D12 should target Lys443 of EGFR, which has a distance of 7.1 Å from the Cα of residue 109 to ε-N atom of Lys443 (FIG. 4F). This distance should not work well for FSK.

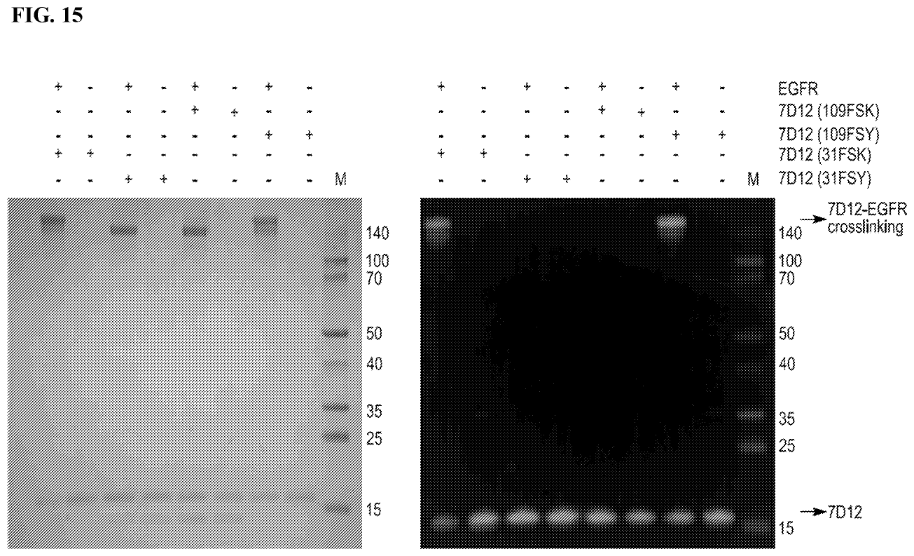

To test these predictions, we incorporated FSK into 7D12 in E. coli and isolated 7D12(30FSK) and 7D12(31FSK) in high purity (FIG. 14). Full-length 7D12 nanobody was obtained only when FSK was added in growth media during expression, and mass spectrometric analysis of the purified nanobody confirmed that only FSK was incorporated (FIG. 4B). We next assessed crosslinking in vitro by incubating 7D12(30FSK) or 7D12(31FSK) with EGFR. SDS-PAGE analysis of the incubated samples showed that the EGFR gel band up-shifted almost completely after incubation with 7D12(31FSK) but not with 7D12(30FSK) or 7D12(WT) (FIG. 4C). This up-shifted band was confirmed to be the covalent crosslinking of EGFR with the nanobody 7D12(31FSK) in Western blot (FIG. 4D). These results indicate that nanobody 7D12(31FSK) crosslinked EGFR in high efficiency. In comparison, at site 109 with a short crosslinking distance, EGFR was crosslinked by 7D12(109FSY) but not by 7D12(109FSK) in SDS-PAGE and Western blot analysis (FIG. 15). Furthermore, we also assessed the ability of 7D12(31FSK) to crosslink native EGFR receptors expressed on cancer cell surface. A431, a human epidermoid carcinoma cell line, was incubated with 7D12(31FSK) or 7D12(WT). Western blot analysis of the cell lysates indicate that 7D12(WT) could not crosslink with the cells, while 7D12(31FSK) covalently crosslinked with EGFR with the crosslinking efficiency increased with time from 1 to 8 h (FIG. 4E). These results demonstrate that FSK and FSY can complement with each other for constructing efficient crosslinking at different reaction distances, allowing the irreversible binding of the nanobody to the EGFR receptor, which will allow for the creation of novel protein-based diagnostics and therapeutics that work in covalent mode.

### Fsk Incorporation and Crosslinking in Mammalian Cells

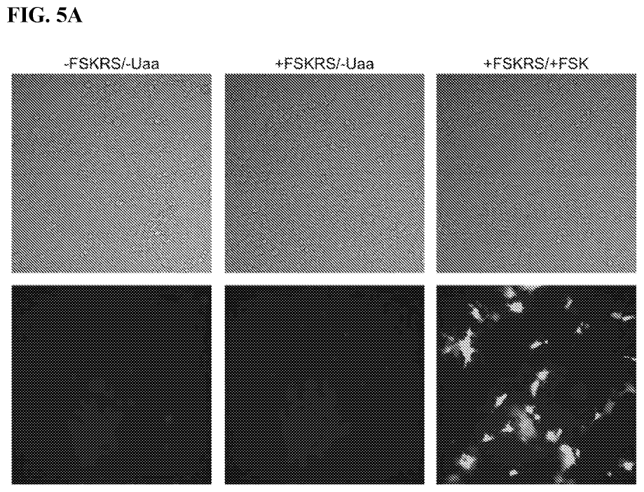

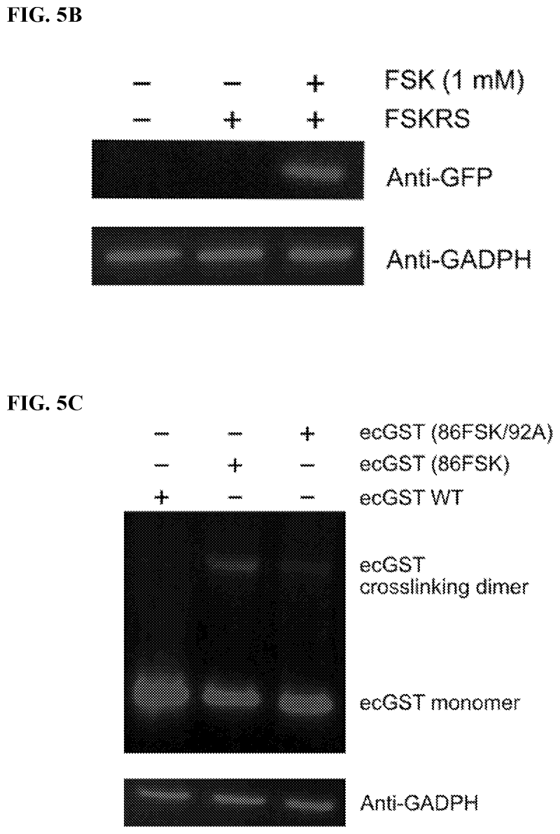

To enable the application of FSK in mammalian cells, we tested FSK incorporation and in vivo crosslinking in human HeLa cells. Plasmid pNEU-FSKRS expressing the FSKRS and tRNA^{Pyl} was transfected into the HeLa-EGFP(182TAG) reporter cells. (Ref. 26). Suppression of the 182TAG codon of the genome-integrated EGFP gene would produce full-length EGFP rendering cells green fluorescent. Strong EGFP fluorescence was observed from cells using confocal microscopy only when FSK as added to the cell culture (FIG. 5A). Western blot analysis of cell lysates using anti-EGFP antibody also showed that full-length EGFP was produced only in cells fed with FSK (FIG. 5B), indicating FSK incorporation into EGFP.

The inventors next explored FSK for protein crosslinking in a mammalian cellular environment. Plasmid pNEU-FSKRS was co-transfected into HEK293T cells with plasmid pCDNA 3.1 expressing GST(WT), GST(86TAG), or GST(86TAG/92A), and cells were grown in the presence of 1 mM FSK. Cell lysates were analyzed with Western blot to detect GST dimeric crosslinking. As shown in FIG. 5C, incorporation of FSK into site 86 of GST successfully lead to GST dimeric crosslinking, which was not observed in the negative controls GST(WT) and GST(86TAG/92A). These results indicate that FSK can be incorporated into proteins in mammalian system using the evolved FSKRS and further used for protein crosslinking in the cells.

Identification of Trx Interactome in E. coli Via FSY or FSK-Mediated Chemical Crosslinking

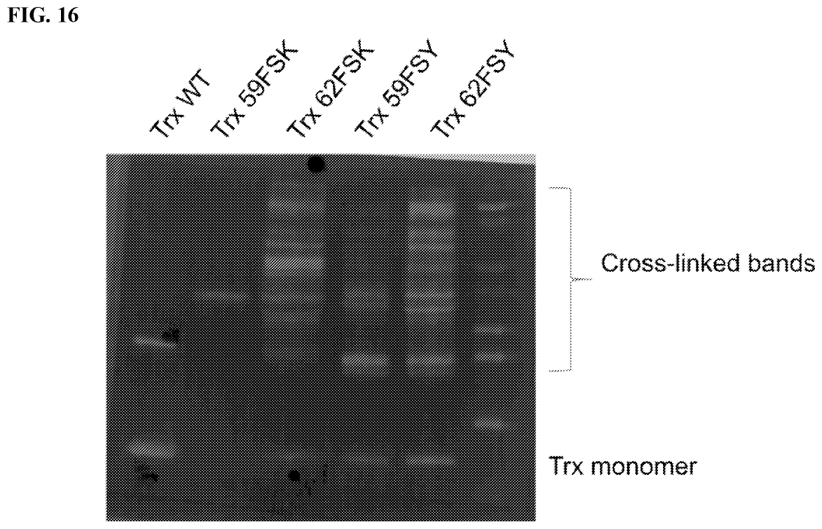

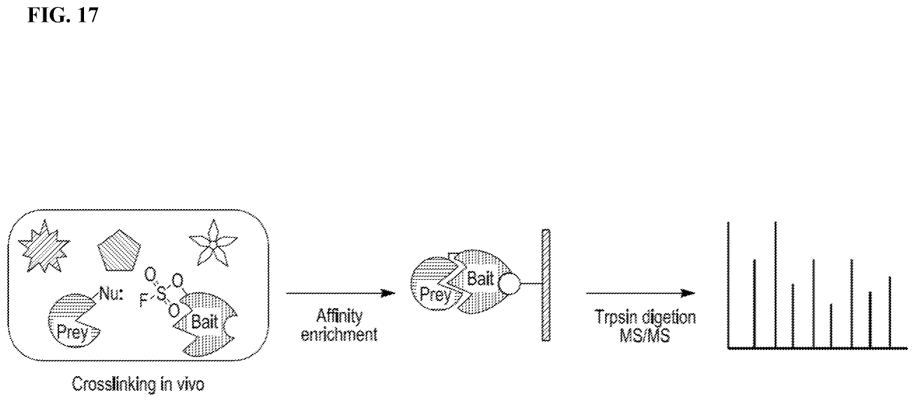

Previously, only haloalkane Uaa was incorporated into the active site of an enzyme to probe its substrate proteins that contain conserved cysteines. Since FSK and FSY have multi-targeting ability toward Lys, His, and Tyr, we reasoned that they can be used to capture a broader range of interacting proteins that lack Cys but have Lys, His, or Tyr at the interaction interface. In addition, FSY and FSK can be incorporated at the peripheral of the binding interface, rather than in the active site or inside the binding interface to minimize potential interference with protein interaction. To further investigate the reaction distance preference of FSK and FSY in a complex protein environment, we explored their application in identifying unknown substrates of an enzyme in live cells via genetically encoded chemical crosslinking (GECX) (FIG. 17A). Specifically, we incorporated FSK or FSY into site 59 or site 62 of thioredoxin (Trx) in E. coli cells. These two sites are away from the Trx active site and likely located in the peripheral of the binding interface of Trx and its substrate. When incorporated into site 62, both FSK and FSY efficiently crosslinked potential substrate proteins, in comparison with the WT Trx (FIG. 16). These crosslinked proteins were pulled down, digested with trypsin, and analyzed with tandem mass spectrometry. Using the OpenUaa software for analysis of crosslinked proteins, we identified 12 substrate proteins for Trx from the Trx(FSK) sample and the Trx(FSY) sample. Among these substrate proteins, AHPC, TPX, SDHA, HPTG, CH10 are well known Trx substrates previously reported. (Ref 27). There are a few overlapped substrates of Trx by using FSK or FSY, such as DNAK, APHC, and TPX However, FSK and FSY showed different residue preference for the same substrate protein AHPC and DNAK Besides these overlapping substrate proteins, FSK and FSY each had its own distinct 9 different substrates (Tables 1-2). These results demonstrate the distinct and complementary value of FSY and FSK in identifying substrates in a complex protein environment.

Table 1 (FSK) and Table 2 (FSY) identify the substrate proteins of Trx and their peptides cross-linked by FSK or FSY, where bold underlined is cross-linked residues, and lower case underlined in SEQ ID NO:18 is Cys alkylated by AM.

TABLE 1
FSK mediated cross-linked substrate of Trx

| Protein | Description | Cross-Linked Peptide | Score |
|---|---|---|---|
| spIP0AC41ISDHA | Succinate dehydrogenase | AAGLHLQESIAEQGALR | 28.58 |
| | flavoprotein subunit | (SEQ ID NO: 4) | |
| | Chaperone protein DnaK | | |
| spIP0A6Y8IDNAK | Alkyl hydroperoxide | IELSSAQQTDVNLPYITADATGPK | 26.30 |
| | reductase C | (SEQ ID NO: 5) | |
| spIP0AE08IAHPC | Adenine | WKEGEATLAPSLDLVGK | 25.84 |
| | phosphoribosyltransferase | (SEQ ID NO: 6) | |
| | Thiol peroxidase | | |
| spIP69503IAPT | 2-iminobutanoate/2- | DVTSLLEDPKAYALSIDLLVER | 14.42 |
| | iminopropanoate deaminase | (SEQ ID NO: 7) | |
| | Chaperone protein HtpG | | |
| spIP0A862ITPX | Deoxyribose-phosphate | SQTVHFCIGNPVTVANSIPQAGSK | 21.72 |
| | aldolase | (SEQ ID NO: 8) | |
| spIP0AF93IRIDA | UDP-3-O-acyl-N- | TIATENAPAAIGPYVQGVDLGNMII | 14.22 |
| | acetylglucosamine | TSGQIPVNPK | |
| | deacetylase Glycine | (SEQ ID NO: 9) | |
| | betaine-binding protein | | |
| | YehZ | | |
| spIP0A6Z3IHTPG | Putative uncharacterized | HIAHDFNDPLTWSHNR | 13.58 |
| | ABC transporter ATP- | (SEQ ID NO: 10) | |
| | binding protein YcjV | | |
| spIP0A6L0IDEOC | Alpha-D-ribose 1- | ASEISIKAGADFIKTSTGK | 10.96 |
| | methylphosphonate 5- | (SEQ ID NO: 11) | |
| | triphosphate synthase | | |
| | subunit PhnL | | |
| spIP0A725ILPXC | Succinate dehydrogenase | LLQAVLAKQEAWEYVTFQDDAEL | 14.22 |
| | flavoprotein subunit | PLAFK | |
| | Chaperone protein DnaK | (SEQ ID NO: 12) | |
| spIP33362IYEHZ | Alkyl hydroperoxide | VAADYLKQK | 10.06 |
| | reductase C | (SEQ ID NO: 13) | |
| spIP77481IYCJV | Adenine | LTIPEEKLAVLK | 10.04 |
| | phosphoribosyltransferase | (SEQ ID NO: 14) | |
| | Thiol peroxidase | | |
| spIP166791PHNL | 2-iminobutanoate/2- | GAAIVGIFHDEAVRNDVADR | 9.40 |
| | iminopropanoate deaminase | (SEQ ID NO: 15) | |
| | Chaperone protein HtpG | | |

TABLE 2
FSY mediated cross-linked substrate of Trx

| | | Cross-linked | |
|---|---|---|---|
| Protein | Description | peptide | Score |
| spIP0A6Y8IDNAK | Chaperone protein DnaK | IELSSAQQTDVNLPY | 27.53 |
| | | ITADATGPK | |
| | | (SEQ ID NO: 16) | |
| spIP0A6Y8IDNAK | Chaperone protein DnaK | AKLESLVEDLVNR | 15.51 |
| | | (SEQ ID NO: 17) | |
| spIP0AE08IAHPC | Alkyl hydroperoxide | AAQYVASHPGEVcPA | 10.91 |
| | reductase C | K | |
| | | (SEQ ID NO: 18) | |
| spIP0AE08IAHPC | Alkyl hydroperoxide | EGEATLAPSLDLVGK | 19.83 |
| | reductase C | I | |
| | | (SEQ ID NO: 19) | |
| spIP0A862ITPX | Thiol peroxidase | SQTVHFQGNPVTVAN | 23.64 |
| | | SIPQAGSK | |
| | | (SEQ ID NO: 20) | |
| spIP0A799IPGK | Phosphoglycerate kinase | SVNDVKADEQILDIG | 22.53 |
| | | DASAQELAEILK | |
| | | (SEQ ID NO: 21) | |
| spIP08312ISYFA | Phenylalanine--tRNA | TMKAQQPPIR | 18.95 |
| | ligase alpha subunit | (SEQ ID NO: 22) | |
| spIP39199IPRMB | 50S ribosomal protein L3 | HEPELGLASGTDGLK | 15.17 |
| | glutamine | LTR | |
| | methyltransferase | (SEQ ID NO: 23) | |
| spIP06720IAGAL | Alpha-galactosidase | TAHIALMDIDPTRLE | 14.22 |
| | | ESHIVVR | |
| | | (SEQ ID NO: 24) | |
| spIP0A6F9ICH10 | 10 kDa chaperonin | VGDIVIFNDGYGVK | 12.40 |
| | | (SEQ ID NO: 25) | |
| spIP0A6X1IHEM1 | Glutamyl-tRNA | EADIIISSTASPLPI | 12.14 |
| | reductase | IGKGMVER | |
| | | (SEQ ID NO: 26) | |
| spIP0AAB8IUSPD | Universal stress | HNDAHLTLIHIDDGL | 22.02 |
| | protein D | SELYPGIYFPATEDI | |
| | | LQLLK | |
| | | (SEQ ID NO: 27) | |

### Discussion

In summary, we developed a new fluorosulfate-containing latent bioreactive Uaa, FSK, for covalent bonding of protein residues with large separations. Where the previously developed multi-targeting bioreactive Uaa FSY could not generate covalent crosslinking due to FSY's shorter reaction radius, FSK enabled efficient covalent linkage via its longer and flexible side chain. This expansion will allow a significantly broader range of protein sites to be covalently connected. In addition to inter-protein crosslinking, FSK could also be used for intramolecular crosslinking, which will greatly expand the diversity of protein crosslinking patterns to facilitate the engineering of novel protein properties. Moreover, we successfully incorporated FSK into nanobodies and converted them into covalent binders for EGFR, which irreversibly bound to EGFR in vitro and on cancer cell surface, which may provide novel avenues for cancer imaging and therapeutics. Finally, we demonstrated that FSK could be incorporated into proteins and generate covalent protein crosslinks in both bacteria and mammalian cells. While sharing the same multi-targeting ability toward His, Lys, and Tyr, FSK complements FSY with a longer and more flexible side chain. Together they are able to offer a powerful latent bioreactive system to create covalent bonds via SuFEx chemistry for almost all proteins and protein-protein interactions. We therefore expect that FSK will find great applications in basic biological studies as well as protein engineering.

### Experimental Procedures

### Reagents and Molecular Biology

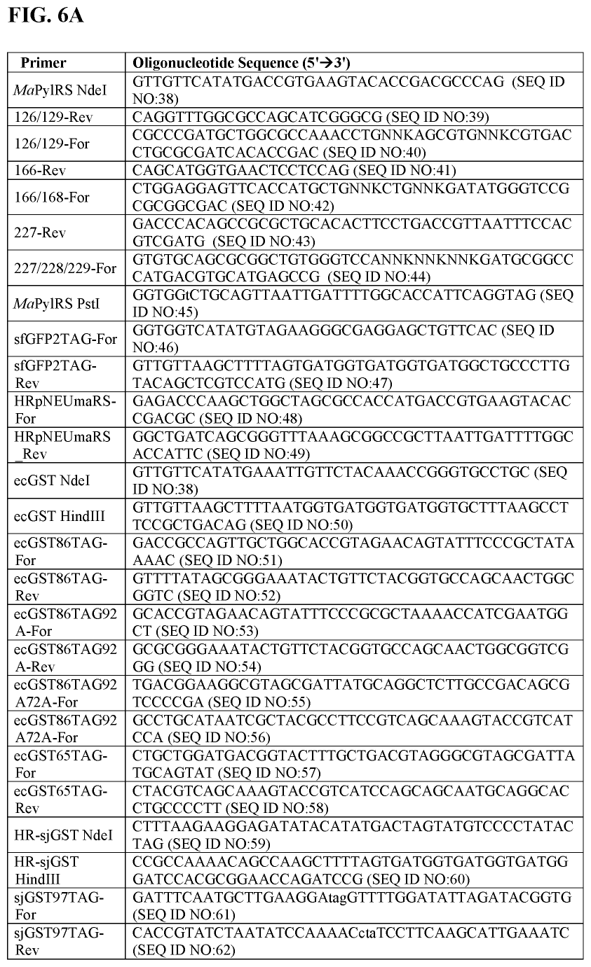

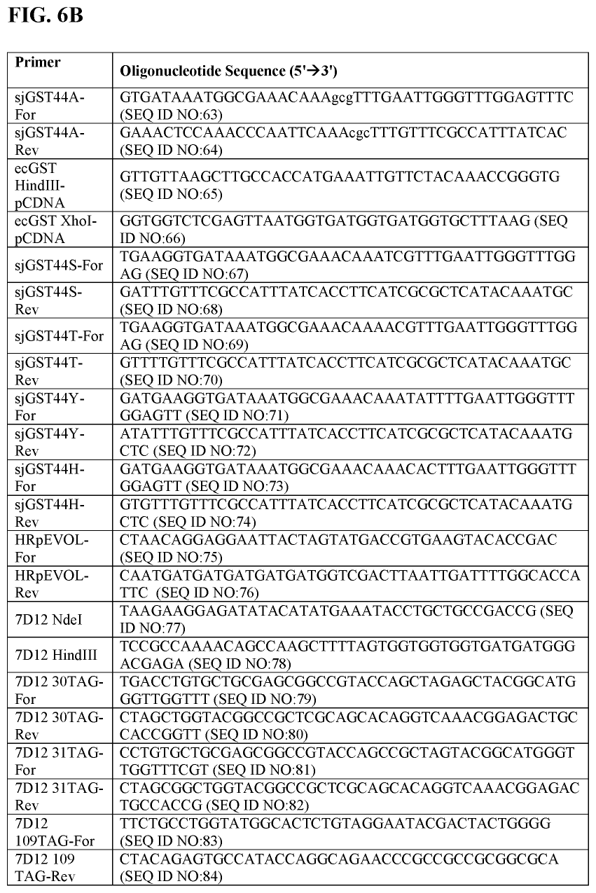

Primers were synthesized and purified by Integrated DNA Technologies (IDT), and plasmids were sequenced by GENEWIZ. All molecular biology reagents were either obtained from New England Biolabs or Vazyme. His-HRP antibody, GFP monoclonal antibodies, GAPDH-HRP antibody were obtained from ProteinTech Group. pBAD-ubiquitin (6TAG) and pBAD-ecGST WT and ecGST mutants were used as previously described. (Liu et al, Journal of the American Chemical Society 2019, 141 (24), 9458-9462). ecGST HindIII-pCDNA and ecGST XhoI-pCDNA primers were used to clone ecGST WT and ecGST (86TAG), ecGST (86TAG/92A), ecGST (86TAG/92A/72A) into pCDNA 3.1. Primers used for cloning are shown in FIG. 6.

FSKRS amino acid sequence is shown as SEQ ID NO: 2.
SEQ ID NO: 2
MTVKYTDAQI QRLREYGNGT YEQKVFEDLA SRDAAFSKEM SVASTDNEKK
IKGMIANPSR HGLTQLMNDI ADALVAEGFI EVRTPIFISK DALARMTITE
DKPLFKQVFW IDEKRALRPM LAPNLGSVAR DLRDHTDGPV KIFEMGSCFR
KESHSGMHLE EFTMLNLFDM GPRGDATEVL KNYISVVMKA AGLPDYDLVQ
EESDVYKETI DVEINGQEVC SAAVGPTPID AAHDVHEPWS GAGFGLERLL
TIREKYSTVK KGGASISYLN GAKIN
sfGFP (2TAG). Primers sfGFP2TAG For and sfGFP2TAG Rev were used to construct
pBAD-sfGFP (2TAG) (SEQ ID NO: 28, where Bold underline: amber codon TAG at 2^{nd}
position)
SEQ ID NO: 28
MXKGEELFTGVVPILVELDGDVNGHKFSVRGEGEGDATNGKLTLKFICTTGKLPVPWP
TLVTTLTYGVQCFSRYPDHMKRHDFFKSAMPEGYVQERTISFKDDGTYKTRAEVKFEG
DTLVNRIELKGIDFKEDGNILGHKLEYNFNSHNVYITADKQKNGIKANFKIRHNVEDGS
VQLADHYQQNTPIGDGPVLLPDNHYLSTQSVLSKDPNEKRDHMVLLEFVTAAGITHGM
DELYKGSHHHHHH
ecGST (86TAG). Primers ecGST NdeI to GST86TAG-Rev were used to construct
pBAD-ecGST (86TAG) by overlap PCR. (SEQ ID NO: 29, where Bold underline: amber codon
TAG at 86^{th} position)
SEQ ID NO: 29
MKLFYKPGACSLASHITLRESGKDFTLVSVDLMKKRLENGDDYFAVNPKGQVPALLLD
DGTLLTEGVAIMQYLADSVPDRQLLAPXNSISRYKTIEWLNYIATELHKGFTPLFRPDTP
EEYKPTVRAQLEKKLQYVNEALKDEHWICGQRFTIADAYLFTVLRWAYAVKLNLEGL
EHIAAFMQRMAERPEVQDALSAEGLKHHHHHH
ecGST (65TAG). pBAD-ecGST (65TAG) was constructed by site-directed
mutagenesis with primers ecGST65TAG-For and ecGST65TAG-Rev (SEQ ID NO: 30, where
Bold underline: amber codon TAG at 65^{th} position)
SEQ ID NO: 30
MKLFYKPGACSLASHITLRESGKDFTLVSVDLMKKRLENGDDYFAVNPKGQVPALLLD
DGTLLTXGVAIMQYLADSVPDRQLLAPVNSISRYKTIEWLNYIATELHKGFTPLFRPDTP
EEYKPTVRAQLEKKLQYVNEALKDEHWICGQRFTIADAYLFTVLRWAYAVKLNLEGL
EHIAAFMQRMAERPEVQDALSAEGLKHHHHHH
ecGST (86TAG/92A). pBAD-ecGST (86TAG/92A) was constructed by site-directed
mutagenesis with primers ecGST86TAG92A-For and ecGST86TAG92A-Rev (SEQ ID NO: 31,
where Bold underline: amber codon TAG at 86^{th} position. Bold Italics: 92 A)
SEQ ID NO: 31
MKLFYKPGACSLASHITLRESGKDFTLVSVDLMKKRLENGDDYFAVNPKGQVPALLLD
DGTLLTEGVAIMQYLADSVPDRQLLAPXNSISRAKTIEWLNYIATELHKGFTPLFRPDTP
EEYKPTVRAQLEKKLQYVNEALKDEHWICGQRFTIADAYLFTVLRWAYAVKLNLEGL
EHIAAFMQRMAERPEVQDALSAEGLKHHHHHH
ecGST (86TAG/92A/72A). pBAD-ecGST (86TAG/92A/72A) was constructed by site-
directed mutagenesis with primers ecGST86TAG92A72A-For and ecGST86TAG92A72A-Rev
(SEQ ID NO: 32, where Bold underline: amber codon TAG at 86^{th} position.
Bold Italics: 72/92 A)
SEQ ID NO: 32
MKLFYKPGACSLASHITLRESGKDFTLVSVDLMKKRLENGDDYFAVNPKGQVPALLLD
DGTLLTEGVAIMQALADSVPDRQLLAPXNSISRAKTIEWLNYIATELHKGFTPLFRPDTP
EEYKPTVRAQLEKKLQYVNEALKDEHWICGQRFTIADAYLFTVLRWAYAVKLNLEGL
EHIAAFMQRMAERPEVQDALSAEGLKHHHHHH
sjGST WT. pBAD-sjGST WT was cloned with primers HR-sjGST NdeI and HR-
sjGST HindIII. (SEQ ID NO: 33)
SEQ ID NO: 33
MTSMSPILGYWKIKGLVQPTRLLLEYLEEKYEEHLYERDEGDKWRNKKFELGLEFPNLP
YYIDGDVKLTQSMAIIRYIADKHNMLGGCPKERAEISMLEGAVLDIRYGVSRIAYSKDF
ETLKVDFLSKLPEMLKMFEDRLCHKTYLNGDHVTHPDFMLYDALDVVLYMDPMCLD
AFPKLVCFKKRIEAIPQIDKYLKSSKYIAWPLQGWQATFGGGDHPPKSDLVPRGSHHHH
HH
sjGST (97TAG) and sjGST (97TAG/44 mutants). pBAD-sjGST (97TAG) and pBAD-
sjGST (97TAG/44A) were constructed by primers HR-sjGST NdeI, sjGST sjGST97TAG-For,
sjGST97TAG-Rev, HR-sjGST HindIII rev, sjGST44A-For, and sjGST44A-Rev. And primers
set 44S-For, 44S-Rev, 44T-For, 44T-Rev, 44Y-For, 44Y-Rev, 44H-For, 44H-Rev were used
to prepare pBAD-sjGST (97TAG/44S), pBAD-sjGST (97TAG/44T), pBAD-sjGST (97TAG/44Y)
and pBAD-sjGST (97TAG/44H). (SEQ ID NO: 34, where Bold underline: amber codon TAG at
97^{th} position. Bold Italics: Paired Lys 44 and its mutation to A, S, T, H, Y.)
SEQ ID NO: 34
MTSMSPILGYWKIKGLVQPTRLLLEYLEEKYEEHLYERDEGDKWRNKKFELGLEFPNLP
YYIDGDVKLTQSMAIIRYIADKHNMLGGCPKERAEISMLEGXVLDIRYGVSRIAYSKDF
ETLKVDFLSKLPEMLKMFEDRLCHKTYLNGDHVTHPDFMLYDALDVVLYMDPMCLD
AFPKLVCFKKRIEAIPQIDKYLKSSKYIAWPLQGWQATFGGGDHPPKSDLVPRGSHHHH
HH
7D12 WT. Primers 7D12 NdeI and 7D12 HindIII were used to clone 7D12 WT to
pBAD plasmid. (SEQ ID NO: 35)
SEQ ID NO: 35
MGQVKLEESG GGSVQTGGSL RLTCAASGRT SRSYGMGWFR QAPGKEREFV
SGISWRGDST GYADSVKGRF TISRDNAKNT VDLQMNSLKP EDTAIYYCAA
AAGSAWYGTL YEYDYWGQGT QVTVSS
7D12 (30TAG). pBAD-7D12 (30TAG) was constructed by site-directed mutagenesis
with primers 7D12 30TAG-For and 7D12 30TAG-Rev (SEQ ID NO: 36, where Bold underline:
amber codon TAG at 30^{th} position.
SEQ ID NO: 36
MGQVKLEESGGGSVQTGGSLRLTCAASGRXSRSYGMGWFRQAPGKEREFVSGISWRG
DSTGYADSVKGRFTISRDNAKNTVDLQMNSLKPEDTAIYYCAAAAGSAWYGTLYEYD
YWGQGTQVTVSS
7D12 (31TAG). pBAD-7D12 (31TAG) was constructed by site-directed mutagenesis
with primers 7D12 31TAG-For and 7D12 31TAG-Rev (SEQ ID NO: 37, where Bold underline:
amber codon TAG at 31^{st} position.)
SEQ ID NO: 37
MGQVKLEESGGGSVQTGGSLRLTCAASGRTXRSYGMGWFRQAPGKEREFVSGISWRG
DSTGYADSVKGRFTISRDNAKNTVDLQMNSLKPEDTAIYYCAAAAGSAWYGTLYEYD
YWGQGTQVTVSS

### Library Construction and FSKRS Mutant Selection

To screen an efficient synthetase for the incorporation of FSK, the primers MaPylRS NdeI to MaPylRS PstI were used to randomize the active site of Methanomethylophilus alvus PylRS-tRNA synthetase (SEQ ID NO:1) and create the library for FSK screening. The selection of an orthogonal synthetase for FSK incorporation was followed the procedure as described previously. (See: Liu et al, Journal of the American Chemical Society 2018, 140 (28), 8807-8816; Liu et al, Angewandte Chemie (International ed. in English) 2018, 57 (39), 12702-12706). Candidate hits were recloned to pEVOL plasmid with primers HRpEVOL-For and HRpEVOL-Rev followed by investigating the incorporation efficiency into pBAD-EGFP (182TAG). The incorporation efficiency for the hits were compared by reading the green fluorescence (excitation at 485 nm, emission at 528 nm) normalized to OD at 600 nm. Four candidate hits were identified, as shown in Table 3 below.

SEQ ID NO: 1
MTVKYTDAQI QRLREYGNGT YEQKVFEDLA SRDAAFSKEM
SVASTDNEKK IKGMIANPSR HGLTQLMNDI ADALVAEGFI
EVRTPIFISK DALARMTITE DKPLFKQVFW IDEKRALRPM
LAPNLYSVMR DLRDHTDGPV KIFEMGSCFR KESHSGMHLE
EFTMLNLVDM GPRGDATEVL KNYISVVMKA AGLPDYDLVQ
EESDVYKETI DVEINGQEVC SAAVGPHYLD AAHDVHEPWS
GAGFGLERLL TIREKYSTVK KGGASISYLN GAKIN

TABLE 3

| Number | Amino Acid Mutations in SEQ ID NO: 1 |
|---|---|
| 1 | Y126G/M129A/V168F/H227T/Y228P/L229I |
| 2 | Y126G/M129A/V168F/H227S/Y228P/L229V |
| 3 | Y126G/M129A/V168F/H227I/Y228P |
| 4 | Y126G/M129A/V168F/H227S/Y228P/L229I |

Incorporation of FSK into EGFP (182TAG), sfGFP (151TAG), sfGFP (2TAG)

pBAD-sfGFP (2TAG), pBAD-sfGFP (151TAG) or pBAD-EGFP (182TAG) was co-transformed with pEVOL-FSKRS into DH10b, and plated on LB agar plate supplemented with 50 μg/mL kanamycin and 34 μg/mL chloramphenicol. A single colony was picked and inoculated into 1 mL 2×YT (5 g/L NaCl, 16 g/L Tryptone, 10 g/L Yeast extract). The cells were left grown 37° C., 220 rpm to an OD 0.5, with good aeration for overnight. Next morning, the cells were diluted 10 times in fresh 2×YT supplemented with relevant antibiotics, 0.2% arabinose with or without 1 mM FSK. The cells were then induced at either 30° C. for 6 hr or 18° C. for overnight. The fluorescence was checked by a plate reader as described above.

General Incorporation of FSK into Proteins for Expression and Purification

For the incorporation of FSK into ubiquitin (6TAG), ubiquitin (18TAG), 7D12 (30TAG), and 7D12 (31TAG), the procedure of transformation is the same as described above. After transformation, a single colony was picked and left grown at 37° C., 220 rpm for overnight. Next morning, the cell culture was diluted 100 times and then regrown to an OD 0.5 in 30 to 100 mL scale, with good aeration and the relevant antibiotic selection. Then the medium was added with 0.2% arabinose with or without 1 mM FSK, and the expression were carried out at 18° C., 220 rpm for 18 hr, 18° C., or 6 hr at 30° C. The IMAC chromatography was used for protein purification. And the procedure was done as described by Liu et al, Journal of the American Chemical Society 2019, 141 (24), 9458-9462.

Utilization of FSK and FSY into ecGST, sjGST and their mutants for protein crosslinking in E. coli.

For probing ecGST or sjGST and their mutants' crosslinking in living E. coli bacterial cells. pBAD-ecGST WT, pBAD-ecGST (86TAG), pBAD-ecGST (65TAG), pBAD-ecGST (86TAG/92A), pBAD-ecGST (86TAG/92A/72A), or sjGST WT, sjGST (97TAG), sjGST (97TAG/44A, S, T, H, or Y) was co-transformed with either pEVOL-FSYRS or pEVOL-FSKRS into DH10b cells. FSY or FSK was added with 0.2% arabinose respectively to the cells for induction when the cells were grown to an OD around 0.5. The cells were grown for protein expression at 37° C. for 6 hr, which then were harvested by centrifugation with a benchtop centrifuge and treated with 2×SDS loading dye containing 100 mM DTT, and boiled for 5 mins at 95° C. The dimerization of GST due to cross linking was monitored by Western blot using anti-his antibody.

Incorporation of FSY or FSK into 7D12

pBAD-7D12 (xxxTAG, xxx indicates the incorporation site) was co-transformed with pEVOL-FSYRS (for FSY incorporation) or pEVOL-FSKRS (for FSK incorporation) into DH10b, and plated on LB agar plate supplemented with 50 μg/mL kanamycin and 34 μg/mL chloramphenicol. After transformation, a single colony was picked and left grown at 37° C., 220 rpm for overnight. Next morning, the cell culture was diluted 100 times and then regrown to an OD 0.5 in 30 to 100 mL scale, with good aeration and the relevant antibiotic selection. Then the medium was added with 0.2% arabinose with or without 1 mM FSY or FSK, and the expression were carried out at 30° C. for 12 hr. The protein purification was carried out with Ni-NTA affinity chromatography.

### In Vitro Crosslinking of 7D12 and EGFR

To explore in vitro crosslinking of 7D12 and EGFR, purified 2 μM 7D12 WT, 7D12(30FSK) or 7D12(31FSK) was incubated with 500 nM recombinant human EGFR protein respectively (Abcam, Cat #ab155726) in 15 μL 1×PBS, pH 7.4. After incubation at 37° C. for 16 h, the samples were treated with a final 1×SDS loading dye and boiled for 5 mins at 95° C. The crosslinking was investigated by running Coomassie blue SDS-PAGE or Western blot with 1:10000 anti-his antibody.

### In Cellular Crosslinking of 7D12 and EGFR

For direct crosslinking of 7D12 to A431 mammalian cells which overexpressed EGFR, A431 cells were seeded in 24-well plate (2×10⁶ cells per well) and cultured overnight at 37° C. The cells were treated with 1 μM 7D12 and 7D12(31TAG) for 1, 2, 4, 8 and 12 h. After digestion with trypsin, the cells were collected by centrifugation at 300 g for 5 min and lysed by adding 100 μL RIPA Buffer with 1× protease inhibitor cocktail. The samples were separated on SDS-PAGE and subjected to Western-blot detection with 1:10000 anti-his antibody. Anti-GAPDH antibody was used as a reference protein.

Genetic Incorporation of FSK into Hela GFP (182TAG)

The plasmid pNEU-FSKRS (1 μg) was transfected into Hela-GFP 182(TAG) cells with 3 μL polyethylenimine (PEI) in 2 mL RPMI 1640 media when the cells population reached 80% confluence. A blank Hela-GFP 182(TAG) cell group was used as a negative control. The cells were treated with or without 1 mM FSK 6 hr after transfection and cultured for additional 48 hr. The cells were washed with 1×PBS for one time and subjected for microscope image after which will be harvested and ran Western blot using anti-GFP antibody. Anti-GAPDH antibody was used as a reference protein.

Genetic Incorporation of FSK into ecGST Mutants in Mammalian Cells

For probing protein crosslinking in mammalian cells. The plasmid pNEU-FSKRS (1.5 μg) was co-transfected with 1 μg pCDNA 3.1 ecGST WT, 1.5 μg ecGST (86TAG), 1.5 μg ecGST(86TAG/92A), and 1.5 μg ecGST(86TAG/92A/72A) respectively into HEK (293T) cells with 9 μL polyethylenimine (PEI) in 2 mL DMEM media when the cells population reached 80% confluence. The cells were treated with or without 1 mM FSK 6 hr after transfection and cultured for additional 48 hr. The cells were harvested and ran Western blot using anti-His antibody. Anti-GAPDH antibody was used as a reference protein.

### Mass Spectrometry

Mass spectrometric measurements were performed as previously described. (Liu et al, Journal of the American Chemical Society 2017, 139 (9), 3430-3437). Briefly for electrospray ionization mass spectrometry, mass spectra of intact proteins were obtained using a QDOT Ultima (Waters) mass spectrometer, operating under positive electrospray ionization (+ESI) mode, connected to an LC-20AD (Shimadzu) liquid chromatography unit. Protein samples were separated from small molecules by reverse phase chromatography on a Waters Xbridge BEH C4 column (300 Å, 3.5 μm, 2.1 mm×50 mm), using an acetonitrile gradient from 30-71.4%, with 0.10% formic acid. Each analysis was 25 min under constant flow rate of 0.2 mL/min at RT. Data were acquired from m/z 350 to 2500, at a rate of 1 sec/scan. Alternatively, spectra were acquired by Xevo G2-S QTOF on a Waters ACQUITY UPLC Protein BEH C4 reverse-phase column (300 Å, 1.7 μm, 2.1 mm×150 mm). An acetonitrile gradient from 5%-95% was used with 0.1% formic acid, over a run time of 5 min and constant flow rate of 0.5 mL/min at RT. Spectrum were acquired from m/z 350 to 2000, at a rate of 1 sec/scan. The spectra were deconvoluted using maximum entropy in MassLynx. For tandem mass spectrometry, analysis and sequencing of peptides were carried out using a Q Exactive Orbitrap interfaced with Ultimate 3000 LC system. Data acquisition by Q Exactive Orbitrap was as follows: 10 NL of trypsin-digested protein was loaded on an Ace UltraCore super C18 reverse-phase column (300 Å, 2.5 μm, 75 mm×2.1 mm) via an autosampler. An acetonitrile gradient from 5%-95% was used with 0.1% formic acid, over a run time of 45 min and constant flow rate of 0.2 mL/min at RT. MS data were acquired using a data-dependent top10 method dynamically choosing the most abundant precursor ions from the survey scan for HCD fragmentation using a stepped normalized collision energy of 28, 30 35 eV. Survey scans were acquired at a resolution of 70,000 at m/z 200 on the Q Exactive. Theoretical patterns of isotopic patterns of peptides were calculated using UCSF MS-ISOTOPE (http://prospector.ucsf.edu) or enviPat Web 2.1 (Loos et al, Analytical chemistry 2015, 87 (11), 5738-5744).

### Example 2

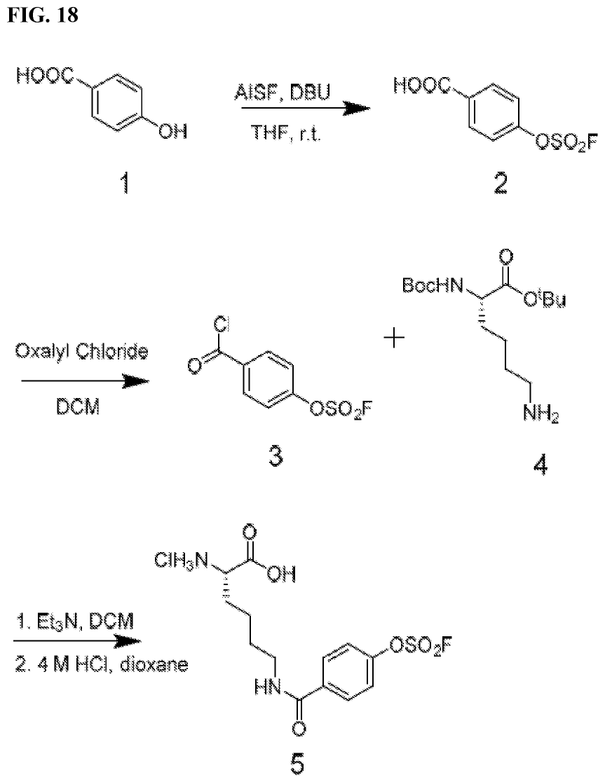

Synthesis of aryl fluorosulfates was based on recent methods to synthesize sulfur (IV) fluorides using [4-(acetylamino)phenyl]imidodisulfuryl difluoride (AISF) reagent. The synthetic scheme for fluorosulfonyloxybenzoyl-L-lysine (5, FSK) is shown in FIG. 18.

Synthesis of 4-((fluorosulfonyl)oxy)benzoic acid (2). To a 200 mL round-bottom flask were added 4-hydroxybenzoic acid (1, 1.38 g, 10 mmol) and [4-(acetylamino)phenyl]-imidodisulfuryl difluoride (AISF) reagent (3.78 g, 12 mmol, 1.2 equiv.). The mixture was dissolved in 50 mL anhydrous tetrahydrofuran and 1,8-diazabicyclo[5.4.0]undec-7-ene (3.35 mL, 22 mmol, 2.2 equiv.) was added dropwise while stirring. The solution was then stirred at r.t. for 20 minutes. The reaction was then diluted with 50 mL ethyl acetate and washed with 1 M HCl (100 mL×2) and brine (100 mL×1). The organic fraction was dried with anhydrous sodium sulfate and concentrated under vacuum. The crude product was then purified by column chromatography using MeOH:CH₂Cl₂ (1:100). The product, 4-((fluorosulfonyl)oxy)benzoic acid, was isolated as a white solid (2, 1.72 g, 7.8 mmol, 78%).

Synthesis of fluorosulfonyloxybenzoyl-L-lysine (5, FSK). To a stirred solution of 4-((fluorosulfonyl)oxy)benzoic acid (2, 0.22 g, 1 mmol) in dry CH₂Cl₂ (15 mL) was added oxalyl chloride (0.21 ml, 2.5 mmol, 2.5 equiv.) dropwise under argon at 0° C. Dimethylformamide (0.1 mL) was then added as catalyst. The reaction mixture was then stirred at r.t. for 5 hours. The solution was then concentrated under vacuum resulting in a yellow oil. The crude 4-(chlorocarbonyl)phenyl sulfofluoridate (3, ˜1 mmol) was redissolved in dry CH₂Cl₂ (10 mL) and cooled to 0° C. N-Boc-Lys-^{t}Bu (4, 0.34 g, 1 mmol, 1 equiv.) was then added, after which Et₃N (0.15 mL, 1.1 mmol, 1.1 equiv.) was added dropwise. The reaction mixture was stirred at r.t. overnight. The reaction was quenched with 20 mL of H₂O and washed with 1 M HCl (20 mL×2). The aqueous phase was combined and extracted with ethyl acetate (20 mL×2). The organic fractions were combined and dried over anhydrous sodium sulfate and concentrated under vacuum. The crude product was then purified by column chromatography using MeOH:CH₂Cl₂(1:100). The product, N-Boc-FSK-^{t}Bu, was isolated as a yellow oil (0.25 g, 0.50 mmol, 50%).

N-Boc-FSK-^{t}Bu (0.25 g, 0.50 mmol) was added to a scintillation vial and dissolved in 4 M HCl in dioxane (10 mL). The reaction was stirred overnight. The resultant solid was filtered off and washed with cool ether (10 mL×2) affording the product FSK-HCl as a white solid (5, 158 mg, 0.41 mmol, 81%)¹H NMR (400 MHz, D₂O): δ (ppm) 7.89 (d, J=8.8 Hz, 2H), 7.59 (d, J=8.8 Hz, 2H), 3.99 (t, J=6.0, 1H), 3.43 (t, J=6.8 Hz, 2H), 2.03-1.94 (m, 2H), 1.72-1.66 (m, 2H), 1.55-1.49 (m, 2H). ¹³C NMR (100 MHz, D₂O): δ (ppm) 173.5, 169.9, 152.4, 135.2, 130.2, 121.9, 53.9, 40.1, 30.2, 28.5, 22.3. HR-ESI (+) m/z: calculated for C₁₃H₁₇FN₂NaO₆S [M+Na]⁺, 371.0684; found 371.0690.

### Example 3

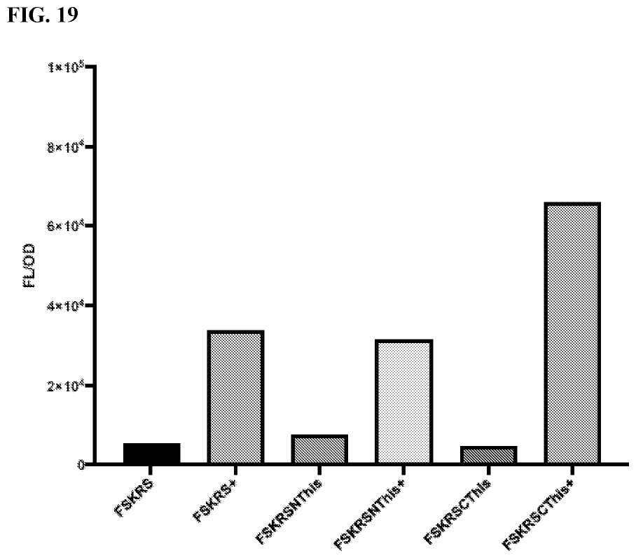

Adding a Hisx6 tag at the C-terminus of FSKRS (SEQ ID NO:2) increased FSK incorporation efficiency by about 96%. Adding the Hisx6 tag at the N-terminus of FSKRS did not increase FSK incorporation efficiency. The increase was robust when cells were cultured at 37° C. The results are shown in FIG. 19, where the FL/OD from left to right are 5410 (FSKRS), 33563 (FSKRS+), 7546 (FSKRSNThis), 31379 (FSKRSNThis+), 4746 (FSKRSCThis), and 65735 (FSKRSCThis+); where FSKRS is SEQ ID NO:2, FSKRS-NThis is SEQ ID NO:86, and FSKRS-CThis is SEQ ID NO:87.

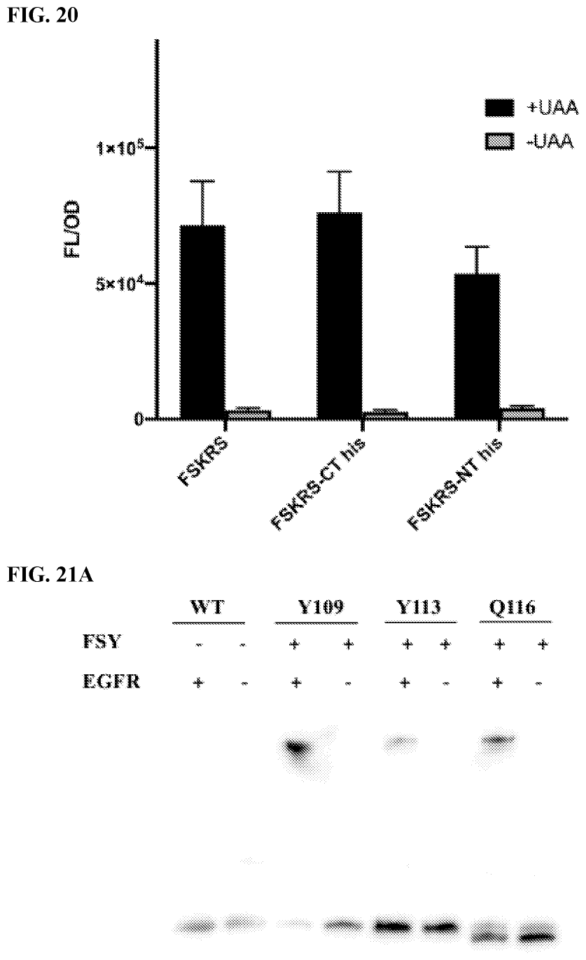

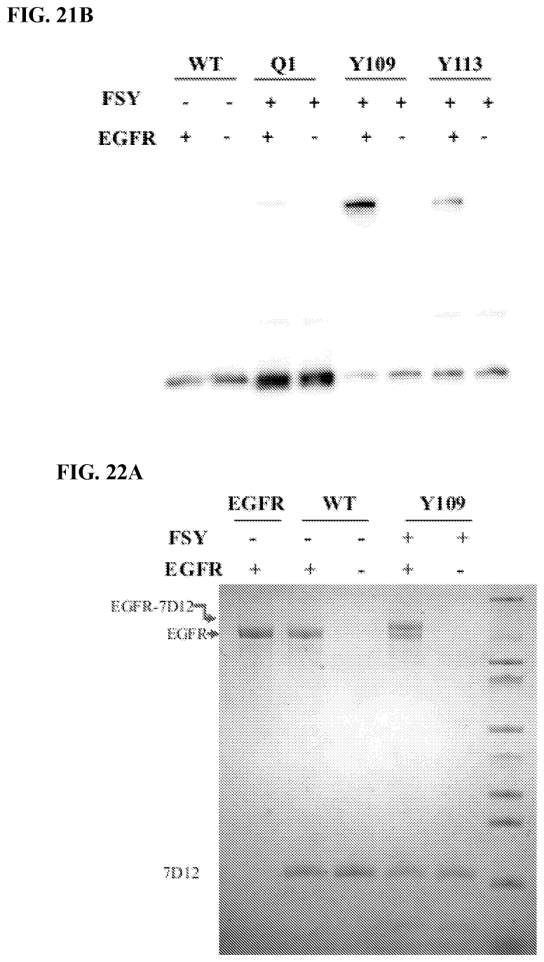

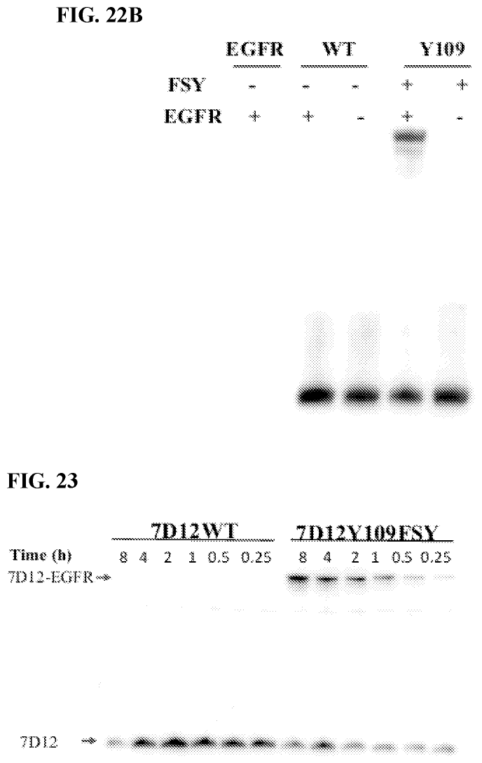

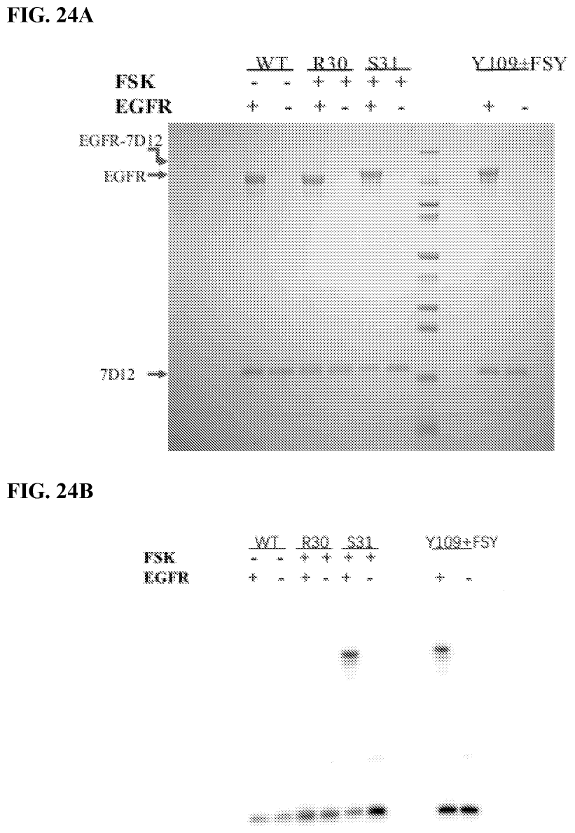

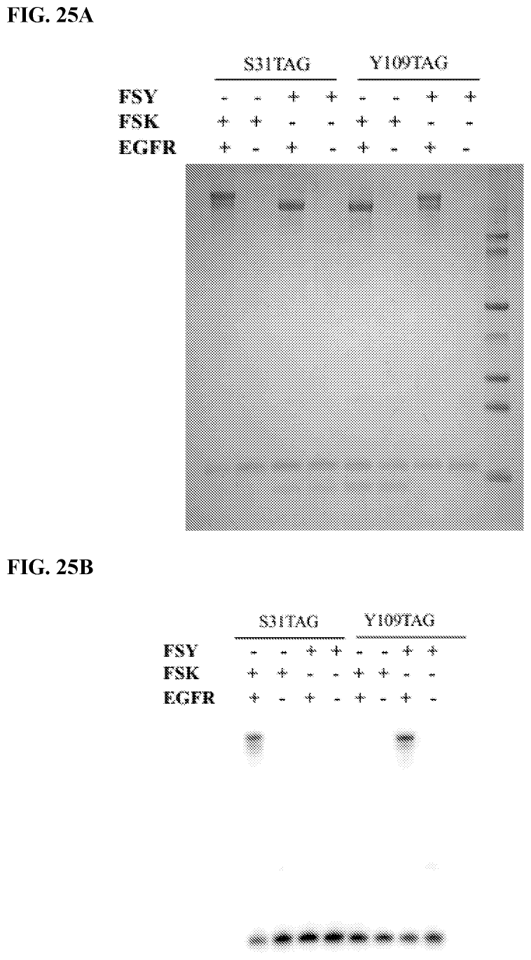

When FSK incorporation was tested at 18° C., the increase in sfGFP fluorescence intensity in the presence of 1 mM FSK was not significant for FSKRS-CTHisx6 over FSKRS, as shown in FIG. 20. With reference to FIG. 20, the FL/OD for +UAA for FSKRS was 71685 and −UAA for FSKRS was 3274; the FL/OD for +UAA for FSKRS-CThis was 76214 and −UAA for FSKRS-CThis was 2602; and the FL/OD for +UAA for FSKRS-NThis was 53687 and −UAA for FSKRS-NThis was 4055.

The fluorescence intensity ratio of +FSK over −FSK was higher for FSKRS-CTHisx6 (29.3 fold) than for FSKRS (21.9 fold), mainly due to a lower background for FSKRS-CTHisx6 in the absence of FSK. The fluorescence intensity ratio of +FSK over −FSK for FSKRS-NTHisx6 was 13.2 fold. Comparison of results at 37° C. and 18° C. indicated the Hisx6 tag appended at C-terminus of FSKRS enhanced the thermostability of the synthetase. Therefore, the increase effect of the Hisx6 tag on FSK incorporation efficiency will be effective at temperatures from about 18° C. to about 37° C. In embodiments, the temperatures are from about 25° C. to about 30° C.

Similar experiments performed with FSYRS to incorporate FSY into sfGFP(151TAG) showed no such effect, suggesting the effect of Hisx6 on FSKRS may be unique. Other tags may have a similar effect on FSKRS.

In summary, appending a Hisx6 tag at the C-terminus of FSKRS increased FSK incorporation efficiency at 37° C.

(FSKRS-NTHis6)
SEQ ID NO: 86
MHHHHHHTVKYTDAQIQRLREYGNGTYEQKVFEDLASRDAAFSKEMS
VASTDNEKKIKGMIANPSRHGLTQLMNDIADALVAEGFIEVRTPIFI
SKDALARMTITEDKPLFKQVFWIDEKRALRPMLAPNLGSVARDLRDH
TDGPVKIFEMGSCFRKESHSGMHLEEFTMLNLFDMGPRGDATEVLKN
YISVVMKAAGLPDYDLVQEESDVYKETIDVEINGQEVCSAAVGPTPI
DAAHDVHEPWSGAGFGLERLLTIREKYSTVKKGGASISYLNGAKIN*
(FSKRS-CTHis6)
SEQ ID NO: 87
MTVKYTDAQIQRLREYGNGTYEQKVFEDLASRDAAFSKEMSVASTDN
EKKIKGMIANPSRHGLTQLMNDIADALVAEGFIEVRTPIFISKDALA
RMTITEDKPLFKQVFWIDEKRALRPMLAPNLGSVARDLRDHTDGPVK
IFEMGSCFRKESHSGMHLEEFTMLNLFDMGPRGDATEVLKNYISVVM
KAAGLPDYDLVQEESDVYKETIDVEINGQEVCSAAVGPTPIDAAHDV
HEPWSGAGFGLERLLTIREKYSTVKKGGASISYLNGAKINHHHHHH*

References: (1) Xiang, Z.; Ren, H.; Hu, Y. S.; Coin, I.; Wei, J.; Cang, H.; Wang, L. Adding an Unnatural Covalent Bond to Proteins Through Proximity-Enhanced Bioreactivity. Nat. Methods 2013, 10 (9), 885-888. (2) Wang, L. Genetically Encoding New Bioreactivity. N. Biotechnol. 2017, 38 (Pt A), 16-25. (3) Xiang, Z.; Lacey, V. K.; Ren, H.; Xu, J.; Burban, D. J.; Jennings, P. A.; Wang, L. Proximity-Enabled Protein Crosslinking Through Genetically Encoding Haloalkane Unnatural Amino Acids. Angew. Chem. Int. Ed. Engl. 2014, 53 (8), 2190-2193. (4) Furman, J. L.; Kang, M.; Choi, S.; Cao, Y.; Wold, E. D.; Sun, S. B.; Smider, V. V.; Schultz, P. G.; Kim, C. H. A Genetically Encoded Aza-Michael Acceptor for Covalent Cross-Linking of Protein-Receptor Complexes. J. Am. Chem. Soc. 2014, 136 (23), 8411-8417. (5) Kobayashi, T.; Hoppmann, C.; Yang, B.; Wang, L. Using Protein-Confined Proximity to Determine Chemical Reactivity. J. Am. Chem. Soc. 2016, 138 (45), 14832-14835. (6) Xuan, W.; Shao, S.; Schultz, P. G. Protein Crosslinking by Genetically Encoded Noncanonical Amino Acids with Reactive Aryl Carbamate Side Chains. Angew. Chem. Int. Ed. Engl. 2017, 56 (18), 5096-5100. (7) Wang, N.; Yang, B.; Fu, C.; Zhu, H.; Zheng, F.; Kobayashi, T.; Liu, J.; Li, S.; Ma, C.; Wang, P. G.; Wang, Q.; Wang, L. Genetically Encoding Fluorosulfate-L-Tyrosine to React with Lysine, Histidine, and Tyrosine via SuFEx in Proteins in Vivo. J. Am. Chem. Soc. 2018, 140 (15), 4995-4999. (8) Liu, J.; Li, S.; Aslam, N. A.; Zheng, F.; Yang, B.; Cheng, R.; Wang, N.; Rozovsky, S.; Wang, P. G.; Wang, Q.; Wang, L. Genetically Encoding Photocaged Quinone Methide to Multitarget Protein Residues Covalently in Vivo. J Am. Chem. Soc. 2019, 141 (24), 9458-9462. (9) Xuan, W.; Collins, D.; Koh, M.; Shao, S.; Yao, A.; Xiao, H.; Garner, P.; Schultz, P. G. Site-Specific Incorporation of a Thioester Containing Amino Acid Into Proteins. ACS Chem. Biol. 2018, 13 (3), 578-581. (10) Hoppmann, C.; Lacey, V. K.; Louie, G. V.; Wei, J.; Noel, J. P.; Wang, L. Genetically Encoding Photoswitchable Click Amino Acids in Escherichia Coli and Mammalian Cells. Angew. Chem. Int. Ed. Engl. 2014, 53 (15), 3932-3936. (11) Hoppmann, C.; Maslennikov, I.; Choe, S.; Wang, L. In Situ Formation of an Azo Bridge on Proteins Controllable by Visible Light. J. Am. Chem. Soc. 2015, 137 (35), 11218-11221. (12) Coin, I.; Katritch, V.; Sun, T.; Xiang, Z.; Siu, F. Y.; Beyermann, M.; Stevens, R. C.; Wang, L. Genetically Encoded Chemical Probes in Cells Reveal the Binding Path of Urocortin-I to CRF Class B GPCR. Cell 2013, 155 (6), 1258-1269. (13) Yang, B.; Tang, S.; Ma, C.; Li, S. T.; Shao, G. C.; Dang, B.; DeGrado, W. F.; Dong, M. Q.; Wang, P. G.; Ding, S.; Wang, L. Spontaneous and Specific Chemical Cross-Linking in Live Cells to Capture and Identify Protein Interactions. Nat. Commun. 2017, 8 (1), 2240. (14) Dong, J.; Krasnova, L.; Finn, M. G.; Sharpless, K. B. Sulfur(VI) Fluoride Exchange (SuFEx): Another Good Reaction for Click Chemistry. Angew. Chem. Int. Ed. Engl. 2014, 53 (36), 9430-9448. (15) Chen, W.; Dong, J.; Plate, L.; Mortenson, D. E.; Brighty, G. J.; Li, S.; Liu, Y.; Galmozzi, A.; Lee, P. S.; Hulce, J. J.; Cravatt, B. F.; Saez, E.; Powers, E. T.; Wilson, I. A.; Sharpless, K. B.; Kelly, J. W. Arylfluorosulfates Inactivate Intracellular Lipid Binding Protein(S) Through Chemoselective SuFEx Reaction with a Binding Site Tyr Residue. J. Am. Chem. Soc. 2016, 138 (23), 7353-7364. (16) Jones, L. H. Emerging Utility of Fluorosulfate Chemical Probes. ACS Medicinal Chemistry Letters 2018, 9 (7), 584-586. (17) Zheng, Q.; Woehl, J. L.; Kitamura, S.; Santos-Martins, D.; Smedley, C. J.; Li, G.; Forli, S.; Moses, J. E.; Wolan, D. W.; Sharpless, K. B. SuFEx-Enabled, Agnostic Discovery of Covalent Inhibitors of Human Neutrophil Elastase. Proc. Natl. Acad. Sci. U.S.A 2019, 116 (38), 18808-18814. (18) Li, Q.; Chen, Q.; Klauser, P. C.; Li, M.; Zheng, F.; Wang, N.; Li, X.; Zhang, Q.; Fu, X.; Wang, Q.; Xu, Y.; Wang, L. Developing Covalent Protein Drugs via Proximity-Enabled Reactive Therapeutics. Cell 2020, 182 (1), 85-97.e16. (19) Liu, J.; Zheng, F.; Cheng, R.; Li, S.; Rozovsky, S.; Wang, Q.; Wang, L. Site-Specific Incorporation of Selenocysteine Using an Expanded Genetic Code and Palladium-Mediated Chemical Deprotection. J. Am. Chem. Soc. 2018, 140 (28), 8807-8816. (20) Nishida, M.; Harada, S.; Noguchi, S.; Satow, Y.; Inoue, H.; Takahashi, K. Three-Dimensional Structure of Escherichia Coli Glutathione S-Transferase Complexed with Glutathione Sulfonate: Catalytic Roles of Cys10 and His106. J. Mol. Biol. 1998, 281 (1), 135-147. (21) Chartron, J.; Shiau, C.; Stout, C. D.; Carroll, K. S. 3′-Phosphoadenosine-5′-Phosphosulfate Reductase in Complex with Thioredoxin: a Structural Snapshot in the Catalytic Cycle. Biochemistry 2007, 46, 3942-3951. (22) Rufer, A. C.; Thiebach, L.; Baer, K.; Klein, H. W.; Hennig, M. X-Ray Structure of Glutathione S-Transferase From Schistosoma Japonicum in a New Crystal Form Reveals Flexibility of the Substrate-Binding Site. Acta Crystallogr Sect F Struct Biol Cryst Commun 2005, 61 (Pt 3), 263-265. (23) Cook, W. J.; Jeffrey, L. C.; Carson, M.; Chen, Z.; Pickart, C. M. Structure of a Diubiquitin Conjugate and a Model for Interaction with Ubiquitin Conjugating Enzyme (E2). J Biol. Chem. 1992, 267 (23), 16467-16471. (24) Chen, X. H.; Xiang, Z.; Hu, Y. S.; Lacey, V. K.; Cang, H.; Wang, L. Genetically Encoding an Electrophilic Amino Acid for Protein Stapling and Covalent Binding to Native Receptors. ACS Chem. Biol. 2014, 9 (9), 1956-1961. (25) Schmitz, K. R.; Bagchi, A.; Roovers, R. C.; van Bergen en Henegouwen, P. M. P.; Ferguson, K. M. Structural Evaluation of EGFR Inhibition Mechanisms for Nanobodies/VHH Domains. Structure 2013, 21 (7), 1214-1224. (26) Wang, W.; Takimoto, J. K.; Louie, G. V.; Baiga, T. J.; Noel, J. P.; Lee, K.-F.; Slesinger, P. A.; Wang, L. Genetically Encoding Unnatural Amino Acids for Cellular and Neuronal Studies. Nat. Neurosci. 2007, 10 (8), 1063-1072. (27) Lu et al, Free. Radic. Biol. Med. 66, 75-87 (2014).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-100. (canceled)

101. A biomolecule comprising fluorosulfonyloxybenzoyl-L-lysine.

102. The biomolecule of claim 101, wherein the biomolecule has the structure of Formula (A):

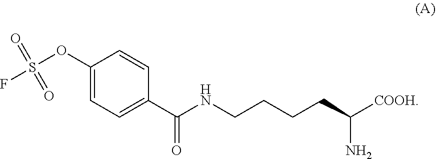 [SMILES: N[C@@H](CCCCNC(=O)c1ccc(OS(=O)(=O)F)cc1)C(=O)O]

103. The biomolecule of claim 101, wherein the biomolecule comprises a protein.

104. The biomolecule of claim 103, wherein the protein has the structure of Formula (B): 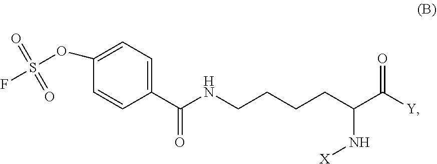
wherein:
(i) X comprises at least one amino acid and Y is OH;
(ii) Y comprises at least one amino acid and X is H; or
(iii) X and Y each comprise at least one amino acid.

105. The biomolecule of claim 103, wherein the protein is an antibody, an antigen-binding fragment, a single-chain variable fragment, a single-domain antibody, or an affibody.

106. The biomolecule of claim 103, wherein the protein is capable of binding to a target.

107. The biomolecule of claim 103, wherein the protein is capable of binding to a target on a surface of a cell.

108. The biomolecule of claim 107, wherein the target on the surface of the cell is a receptor.

109. The biomolecule of claim 108, wherein the receptor comprises a membrane receptor or a hormone receptor.

110. The biomolecule of claim 106, wherein the target is PD-1 or PD-L1.

111. The biomolecule of claim 106, wherein the protein forms a covalent bond with the target when the target is bound by the biomolecule.

112. A cell comprising the biomolecule of claim 101.

113. The cell of claim 112, wherein the cell is a bacterial cell, a fungal cell, a plant cell, an archaea cell, or an animal cell.

114. A pharmaceutical composition comprising the biomolecule of claim 101.

115. A method of forming a biomolecule conjugate, the method comprising contacting the biomolecule of claim 101 with a second biomolecule moiety; wherein the second biomolecule moiety is reactive with the fluorosulfonyloxybenzoyl-L-lysine; thereby forming the biomolecule conjugate.

116. The method of claim 115, wherein the biomolecule conjugate has the structure of Formula (I), Formula (II), or Formula (III): 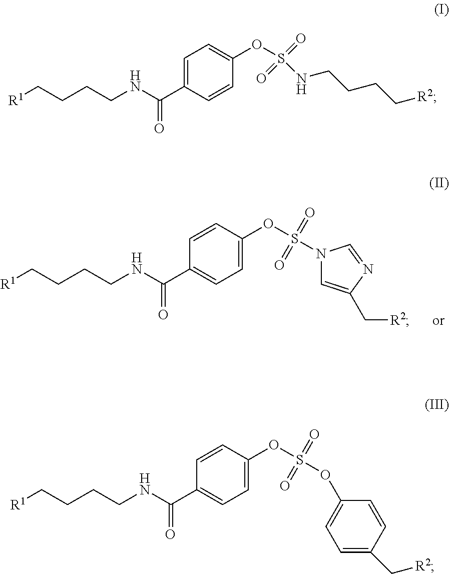
wherein R¹ is the biomolecule and R² is the second biomolecule moiety.

117. The method of claim 115, wherein the contacting is performed in vivo.

118. A method of producing the protein of claim 104, the method comprising contacting a nucleic acid with a pyrrolysyl-tRNA synthetase, a tRNA^{Pyl}, and a fluorosulfonyloxybenzoyl-L-lysine; wherein the nucleic acid encodes a protein, and wherein the nucleic acid comprises at least one codon recognized by the tRNA^{Pyl}, thereby producing the protein.

119. The method of claim 118, wherein the pyrrolysyl-tRNA synthetase comprises one or more amino acid residues substitutions within the substrate-binding site of the pyrrolysyl-tRNA synthetase having the amino acid sequence of SEQ ID NO:1; wherein the substitutions in the amino acid sequence of SEQ ID NO:1 are selected from the group consisting of (i) Y126G; (ii) M129A; (iii) V168F; (iv) H227T, H227S, or H227I; (v) Y228P; and (vi) L229I, L229V, or L229I.

120. A pyrrolysyl-tRNA synthetase comprising one or more amino acid residues substitutions within the substrate-binding site of the pyrrolysyl-tRNA synthetase having the amino acid sequence of SEQ ID NO:1; wherein the substitutions in the amino acid sequence of SEQ ID NO:1 are selected from the group consisting of (i) Y126G; (ii) M129A; (iii) V168F; (iv) H227T, H227S, or H227I; (v) Y228P; and (vi) L229I, L229V, or L229I.
